SEQUENCE LISTING (5 sequences)

SEQ ID NO: 1 - DNA, 3412 bp, Homo sapiens
agccgagcagcctggcaacggcggtggcgcccggagcccgagagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaggggggaccctggcaccgcattagagctcgaaataaaggcgatagctgactttcatttggggcatttgtaaaaagtagattaaaatatttgcctccatgtagaacttgaactaacataatcttaaactcttgaatatgtgccttctagaatacatattacaagaaaactacagggtccacacggcaatcagaagaaaggagctgagatgaggttttggaaaaccctgacacctttaaaaagcagtttttgaaagacaaaatttagatttaatttacgtcttgagaaatactatatatacaatatatattttgtgggcttaattgaaacaacattattttaaaatcaaaggggatatatgtttgtggaatggattttcctgaagctgcttaacagttgctttggattctctaagatgaatccaaatgtgaaagatgcatgttactgccaaaaccaaattgagctcagcttcctaggcattacccaaaagcaaggtgtttaagtaattgccagcttttataccatcatgagtggtgacttaaggagaaatagctgtatagatgagtttttcattatttggaaatttaggggtagaaaatgttttcccctaattttccagagaagcctatttttatatttttaaaaaactgacagggcccagttaaatatgatttgcattttttaaatttgccagttttattttctaaattctttcatgagcttgcctaaaattcggaatggttttcgggttgtggcaaaccccaaagagagcactgtccaaggatgtcgggagcatcctgctgcttaggggaatgttttcgcaaatgttgctctagtcagtccagctcatctgccaaaatgtagggctaccgtcttggatgcatgagctattgctagagcatcatccttagaaatcagtgccccagatgtacatgtgttgagcgtattcttgaaagtattgtgtttatgcatttcaatttcaatggtgttggcttcccctccccaccccacgcgtgcataaaaactggttctacaaatttttacttgaagtaccaggccgtttgctttttcaggttgttttgttttatagtattaagtgaaattttaaatgcacagttctatttgctatctgaactaattcatttattaagtatatttgtaaaagctaaggctcgagttaaaacaatgaagtgttttacaatgatttgtaaaggactatttataactaatatggttttgttttcaatgaattaagaaagattaaatatatctttgtaaattattttatgtcatagtttaattggtctaccaagtaagacatctcaaatacagtagtataatgtatgaattttgtaagtataagaaattttattagacattctcttactttttgtaaatgctgtaaatatttcataaattaacaaagtgtcactccataaaaagaaagctaatactaatagcctaaaagattttgtgaaatttcatgaaaactttttaatggcaataatgactaaagacctgctgtaataaatgtattaactgaaacctaa

SEQ ID NO: 2 - protein, 603 aa, Homo sapiens
MASASTSKYNSHSLENESIKRTSRDGVNRDLTEAVPRLPGETLITDKEVIYICPFNGPIKGRVYITNYRLYLRSLETDSSLILDVPLGVISRIEKMGGATSRGENSYGLDITCKDMRNLRFALKQEGHSRRDMFEILTRYAFPLAHSLPLFAFLNEEKFNVDGWTVYNPVEEYRRQGLPNHHWRITFINKCYELCDTYPALLVVPYRASDDDLRRVATFRSRNRIPVLSWIHPENKTVIVRCSQPLVGMSGKRNKDDEKYLDVIRETNKQISKLTIYDARPSVNAVANKATGGGYESDDAYHNAELFFLDIHNIHVMRESLKKVKDIVYPNVEESHWLSSLESTHWLEHIKLVLTGAIQVADKVSSGKSSVLVHCSDGWDRTAQLTSLAMLMLDSFYRSIEGFEILVQKEWISFGHKFASRIGHGDKNHTDADRSPIFLQFIDCVWQMSKQFPTAFEFNEQFLIIILDHLYSCRFGTFLFNCESARERQKVTERTVSLWSLINSNKEKFKNPFYTKEINRVLYPVASMRHLELWVNYYIRWNPRIKQQQPNPVEQRYMELLALRDEYIKRLEELQLANSAKLSDPPTSPSSPSQMMPHVQTHF

SEQ ID NO: 3 - DNA, 1060 bp, Artificial Sequence
  Synthetic Construct
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 4 - DNA, 1822 bp, Artificial Sequence
  Synthetic Construct
agtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctga

SEQ ID NO: 5 - DNA, 12471 bp, Artificial Sequence
  Synthetic Construct
tcgcgcgtttcggtgatgacggtgaaaacctctgacacatgcagctcccggacgtcattgtcgatcctgcaggcgtacggtaaaaaaaggcatagctaacaaggtgtggaaaaagaattagtggttagagagtgagctattcgttgaaacaattgcgttcttgaaacaattcttgctggtaaaatgtcacattttatgtgactacaggtggaggattggcacataacctaaccagtgggggaaacaattgacctctggatttgtccaagtgtatagtagcatttgcccaatcgaatggtcctggtaaggtgttaatgttgactagaaccaaaggtggaagttgcagggaaactggtttagtacaagggtggacaccaggcagtcatccagaggcccattaaaggccttggaatgtttttccgaaggagaatcactccctcttctctcgcttaaagttttaggggattcatgaacagctgctgtgggatagtttcatgtccctagcaattgtaaagcaactgagggtggcttaaaccagttttagctttagggttagggttactggactaaaatttgagaaattcataaatcttaaggaaatccattgtgagttttcattatgagtgcatccaatgtataatttccatgaccctcccatgcaagtgagcatgtgaatcaggaaacgttacaagaacccaacaaactcaaccactactagacaggcgatcacttccagttagtatgcaactttctgtgtaattttagttaccattaaaatctggatgaccttagtgtaaggaaaaaataccttgaatagtgttaaagatgtacacttggtgtcaggcattgtaacattgataaatctgtgtaaggtgctttttgaaaacttcaaagctgcatcaagtcaagtacaagaaaggccatggctgctaaagctgttgaagatgtgggatggaactgggtcacattggtgttaacagcgttgtgcagagccggcaggatcttggtgtgagcgaacattagtctatttaataaagctgtgtgaatgttgtagaggtgaggatgctcacttgaaaactcactgaagaacacttggccccttgaactaaagtgcttctatcaagttcagtgagaaattccgaattacaagcataggtactagaaaagttttgaaaagcagtatagagcaacataagcacattcataaaattagtgatgtagaaagtgaaatttccacgtatggtcactcccagagaaaaaaaatacgtttatttaccttttttaaaaataggggatttcaggccgggtgaggtggctcacgcctgtaatcccagcactttgggaggcccaggtgggcggatcacctgaggtcaggagttggagggatggcaaatcccatctctacaaaatatacaaaaaaatagctgggtgtgttggcaggcgcctgtaatcccagctactcggaaggctgaggcaggagaatccctggaaccagggatgtggaggttgcagtgagccgagattgtgtaactgcattccagcctgggcaacaagagcaagactccgtatcaggaaaaaaaaaagggggggttggatttcgcttgttgcataggttggtctcaaactcctggcctcaagtgattctcctgcctctgcctcccaaagtgctgagattacaggtgtgaggcaccatgccaggtctcttactgtttgtaattaaatacatacacattttgtgtgtttgtgtgcacctttataaagtcaaaggtgatagtaacccatttaagttcctactcaattttactttccagggataactaactactttttctttttgagatggagtctcgctgtgtagcccaggctggagtgcagtggcaccatctcggctcactgcaagctcctcctccctggttcacgctattctcctgcctcagcctccccaacaactaggactacaggctcacctcgccatacctggctaattttttgtatttttagtagagacagggtttcactgtgttagccaggatggtctcgatctcctgaccttgtgatccgcctgcctctgcctcccaaagtgctgggattacaggcatgagcaacctcacccagctgggataactactttttacaggttgatattcttttggacttttcccctgtgtaaaaatatactatatttgttatgtacatattatgtacatacagacacaaattggaccattctcagtataatgattctcaggttttttttttttttttgaggtggggaactagataattatggacatctttccatactagcatatcaatatctacctcattctttttaatatttttgctagtattccattgtatgaatgtcctatgatttacttaacctgtccatcaatatttgtttccaggtttttgctattataatgctgctgcaaagtacatcctcacacatctttattttgtctattcatatttctgtaagataggttactaaagttggaactgccaaattaacactatcatactattttgttttttaattttaattttttaaaaaatgtaaaatgtgcaatttcaagaggagaaacttgaacacaaggagcaaaatctatttttataacatcctattaaaagcttgctttacataaagattttgaaagaatagcataaatacaagatttctattttaattggattcttagggctaataaaataatcagccttagcacttatttatttattttttttgagagggagtctcgctctgttgtccatgctggagtgcagtggcgtgatctcggctcactgcaagctccacctcatgagttcacaccattctcctgcctcagtctcccgagtagctgggactccaggcgccctctacaaagcccgtctaattttttttgtatttttagtagagacagggtttcactgtgttagccaggatggtcttgatctcctgaccttgtgatctgcccgcctcggcctcccaaagtgctgggattataggcttgagccactgctcccggccagcacttatttttataattcttcatgattactgtgttactgtcccatgggccgccagggccagctaggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcctagcacgcgctaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccggactctagaggatccagatctaagcttctctggtcaccgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcaagtttccaggatggcttctgcatcaacttctaaatataattcacactccttggagaatgagtctattaagaggacgtctcgagatggagtcaatcgagatctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgaccggtccgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgaccggccgctctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctagaggccgccagggccatatttctcaatttttaaatttttcaaaaaaattaatccttaatgtgcatatttttgaattgttaatataactttttgaggtgatgtcttcatgtgtttcaactacttaaaaacttttaaacagtatataataaaaaatcttccaggccactcacacctgtaatcccagcactttgggaggctgaggtgggcagatcacctgagggcaggagttcgagaccagcctggccaatatatatatattcatatattcatatatatatatatattcatatattcatatatatatattcatatattcatatatatatatatatatatatatagcaaaacctcatctctaataaaatacaaaaattagctgagcgtggtgatggatgcctgtagtcccagctactcgggaggctgaggcaggagaatctcttgaacctgggaggtggaggttgcagtgagctgagatggtgccactgccctccagcctgagtgacagagcgagactcggtctccaaaaaaaaacaacaaaaaaatcttccatccttgtctcccatccaccccttccccccagcatgtacttgcagactttatgcatatacagtgagtactgtatatacacaaataataaaaaaatcatatatataatatatgtaattcccctttacatgaaaggtagcacactggtctgtacagtctgtctgcactgtgctatttcactttatatttttatagtttgacagagttctaacatttctttttttttttttttaacagagtcttgttcctgattgttaaattttaaagcatcctaaagtttggtttcacacttgaatgaataccatgtaaggattcacttacatagatgtggttgcctgaatcttaagaataaaataacattgtttgtatttatttaaattagtgttccttttatggtttgcctgaaagcacaacaaaatcctcaccaagatattacaattatgactcccatacaggtaaactgtttagagattggcaagcaccttttaatgaaaggagtcagccagcttagtgtgcagtatttatttctgccggaagagggagcttcagggacagactttggtttagtcatgaagcctccagcactcccaagcggttgtggttgaccaagcaatttatgcttttacctttctacttccagaggcttgtttacttatcagtaagcattaatttagtgtcccctcagatgccttttactttcttcttttctgcctagaataagctgctcttccaattttgcagctacatgtttccaccccagttggaatttctccataacatccattgtagctatccttcaatctacagcctctatttcctgttatagctggtcaggtctaatccctcaaaatactctgtcccctgcttcccttatctgctggccacctttttcccccacatacacactgccatgtcccacccttcactcaagttgttccctgccacctcaacaaatttaagtccataaaatagagtaagtgttcctgactgttaaattttaaagcatcccaaagtctgatttcacactcgaatgaatactatgtacggattcatttacatagatgcggttgcatgagtcttaacaaaaaaataacattatttgtatttattcaaagtactgtcaagatataatgtcaagacctaattcaaaggttccacaaagccttccttgactgcccccaacgaagattatccattttccctgaaatcccattgacttttctattttgtaaggaggctcgtgagactctgtctaaaaacaaaacaaaacaaaaagaaacaatcaaacggcttgcttctgttctttgatctgctagtaagcaaaaattacacatggtgacaggagctatgtgaggctgtcaggttgaatgggaggagtttgggatcctgcttgtggatggttggaagaggctttcgggaaagacagtatttatgtgagacctggaagatgggccttagctttgcagaaggtggagaggcaggaaatagcacgggggccctggggctggaagacttgggcatatttgaggaacagaaaggagaccagcataactgaggtgggaaaagcatgtgaagagatggggctggaggaggccgggagtggtggctcacgcctgtaatcccagcactttgggaggccaaggcaggcggatcatgagctcaggagattgagaccatcctggctaacacggtgaaaccccctctctactaaaaatacaaaaaaaaaaaaaaaaaaaattagctgggcgtggtggcaggagcctgtagtcccagctacctgggaggctgaggcaggagaatggcgtgaacctggaaggctgagcttgcagtgagccgagattgcaccactgcactccagcctgggagacagagagagactccctctcaaaaaaacaaacaaacgaaacaaaacaaaacaaaaattagccaggcgtggtggtatgcacctgtaatcccagctactcgggaggttgaggcaggagaaacgcttgaactcaggaggcggaggttgcagtgagccgagactgcgccactgcactccagcctgggtgacagagggagactccatctcaaaaaaaaaaatttttttttttttacaaacggtgtctccctctgtcgcccaggctggagtgcagtggtgtgatcacagctcactccagcctcaacctccccagctgaagccatcctcttgcctcagcctcctaagtagctgggactacaggcgcgcacctccaggcttggctcttattctttttattgtttttgaaactatagaacctatttttaaaaaatgttttggttgtttttattgctgcttttccttttggggttagaacacaagttttgatgggaaacaggttagaacacattcatctcttcccatagcgatggtcatagaaaaacggggcatatttataaactctcagttgatcttaaaatgtgcaaaagctgccgaactcctgggagtgagctcgagccctgcaggatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

(19) United States
(12) Patent Application Publication
(10) Pub. No.: US 2024/0148906 A1
(43) Pub. Date: May 9, 2024

(54) COMPOSITIONS AND METHODS FOR TREATING X-LINKED MYOTUBULAR MYOPATHY

(71) Applicant: AUDENTES THERAPEUTICS, INC., San Francisco, CA (US)

(72) Inventors: Geovanny PEREZ, Amherst, NY (US); Robert GRAHAM, Newton, MA (US); Salvador RICO, Berkeley, CA (US); Suyash PRASAD, Woodacre, CA (US)

(21) Appl. No.: 18/281,346

(22) PCT Filed: Mar. 11, 2022
(86) PCT No.: PCT/US2022/019876

§ 371 Date: Sep. 11, 2023

Related U.S. Application Data
(60) Provisional application No. 63/159,898, filed on Mar. 11, 2021.

(51) Int. Cl. A61K 48/00; A61M 16/00; A61P 21/00

(52) U.S. Cl. CPC A61K 48/0058; A61M 16/024; A61P 21/00; A61M 2205/3306; A61M 2205/3327; A61M 2230/20; A61M 2230/42; A61M 2230/43; A61M 2230/63

(57) ABSTRACT

The present invention provides compositions and methods for treating a neuromuscular disorder. In certain embodiments, the invention provides compositions and methods for assessing weaning readiness and continuation of weaning off of a mechanical ventilator in a subject with X-linked myotubular myopathy (XLMTM).

### SEQUENCE LISTING

The instant application contains a Sequence Listing which has been submitted electronically in ASCII format and is hereby incorporated by reference in its entirety. The ASCII copy, created on Mar. 8, 2022, is named “51037-056WO2_Sequence_Listing_3_8_22_ST25” and is 30,386 bytes in size.

### FIELD OF THE INVENTION

The present invention generally relates to a method for the treatment of neuromuscular disorders. More particularly, the present invention relates to a system of parameters and a method for assessing the readiness of a patient for the initiation or continuation of weaning off of a mechanical ventilator.

### BACKGROUND OF THE INVENTION

X-linked myotubular myopathy (XLMTM) is a fatal monogenic disease of skeletal muscle, resulting from loss-of-function mutations in Myotubularin 1 (MTM1) (Laporte et al., 1996, Nat Genet 13(2):175-82). Approximately one in every 50,000 newborn boys has XLMTM, which typically displays as marked hypotonia and respiratory failure (Jungbluth et al., 2008, Orphanet J Rare Dis 3:26). In extremely rare cases, females can develop a severe form of XLMTM. Survival beyond the postnatal period requires intensive support, including respiratory support (i.e., mechanical ventilation) at birth in 85-90% of patients, ongoing 24-hour ventilator dependence in 48% of patients, and tracheostomy in 60% of patients. No effective precedent for the weaning of chronically ventilated patients with congenital neuromuscular diseases off of mechanical ventilation exists.

Thus, there is a need in the art for methods for assessing a patient's readiness for weaning initiation or the continuation of weaning off of a mechanical ventilator. The present invention satisfies this unmet need.

### SUMMARY OF THE INVENTION

This disclosure provides methods for assessing readiness for weaning initiation or the continuation of weaning off of a mechanical ventilator in a patient for the treatment of diseases and disorders related to loss-of-function mutations in Myotubularin 1 (MTM1). In some embodiments, the disorder is X-linked myotubular myopathy (XLMTM).

In one aspect, the disclosure provides a method of weaning a human patient that is on mechanical ventilation and that has XLMTM off of mechanical ventilation, wherein the patient has previously been administered a therapeutically effective amount of a viral vector including a transgene encoding MTM1, the method including: determining that the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a saturation of room air oxygen (SpO₂) of about 94% or more, (v) a transcutaneous CO₂ (TcCO₂) of from about 35 mmHg to about 45 mmHg, (vi) an End Tidal CO₂ (petCO₂) of from about 35 mmHg to about 45 mmHg, and (vii) a serum bicarbonate level of from about 22 mEq/L to about 27 mEq/L; and weaning the patient off of mechanical ventilation during daytime hours.

In some embodiments of the foregoing aspect, the method includes determining that the patient exhibits a maximal inspiratory pressure of about 50 cmH₂O or more on a ventilator.

In some embodiments of any of the foregoing aspects, the method includes determining that the patient exhibits a maximal expiratory pressure of about 40 cmH₂O or more on a ventilator.

In some embodiments of any of the foregoing aspects, the method includes determining that the patient exhibits a positive end-expiratory pressure of about 5 cmH₂O or less on a ventilator.

In some embodiments of any of the foregoing aspects, the method includes determining that the patient exhibits a SpO₂ of about 94% or more.

In some embodiments of any of the foregoing aspects, the method includes determining that the patient exhibits a TcCO₂ of from about 35 mmHg to about 45 mmHg.

In some embodiments of any of the foregoing aspects, the method includes determining that the patient exhibits a petCO₂ of from about 35 mmHg to about 45 mmHg.

In some embodiments of any of the foregoing aspects, the method includes determining that the patient exhibits a serum bicarbonate level of from about 22 mEq/L to about 27 mEq/L.

In some embodiments of any of the foregoing aspects, the method further includes determining that the patient exhibits vital signs and a weight that are within age-adjusted norms.

In some embodiments of any of the foregoing aspects, the method further includes determining that the patient exhibits a motor function score on the Children's Hospital of Philadelphia Infant Test of Neuromuscular Disorders (CHOP INTEND) of greater than 45 or that neuromuscular development milestones have been met.

In some embodiments of any of the foregoing aspects, the method further includes: determining that the patient exhibits one or more of (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an apnea-hypopnea index (AHI) of less than 5 events/hour as assessed by polysomnogram (PSG) performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a partial pressure of CO₂ (ptcCO₂) of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with an tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TcCO₂ of greater than 45 mmHg as assessed by a video recording of a respiratory sprinting trial, and (xvi) a TCO₂ that does not increase by 10 mmHg or more relative to the patient's awake baseline as assessed by a video recording of a respiratory sprinting trial; and continuing to wean the patient off of mechanical ventilation during daytime hours.

In some embodiments of any of the foregoing aspects, the method includes determining that the patient exhibits a TcCO₂ of from about 35 mmHg to about 45 mmHg as assessed by nocturnal respiration monitoring.

In some embodiments of any of the foregoing aspects, the method includes determining that the patient exhibits a petCO₂ of from about 35 mmHg to about 45 mmHg as assessed by nocturnal respiration monitoring.

In some embodiments of any of the foregoing aspects, the method includes determining that the patient exhibits an SpO₂ of about 94% or more as assessed by nocturnal respiration monitoring.

In some embodiments of any of the foregoing aspects, the method includes determining that the patient exhibits an AHI of less than 5 events/hour as assessed by PSG performed with an open tracheostomy.

In some embodiments of any of the foregoing aspects, the method includes determining that the patient exhibits a TcCO₂ of from about 35 mmHg to about 45 mmHg as assessed by PSG performed with an open tracheostomy.

In some embodiments of any of the foregoing aspects, the method includes determining that the patient exhibits a TcCO₂ that does not increase by 10 mmHg or more relative to the patient's awake baseline as assessed by PSG performed with an open tracheostomy.

In some embodiments of any of the foregoing aspects, the method includes determining that the patient exhibits a petCO₂ or a ptcCO₂ of less than 50 mmHg as assessed by PSG performed with an open tracheostomy.

In some embodiments of any of the foregoing aspects, the method includes determining that the patient exhibits a petCO₂ or ptcCO₂ that does not increase by 10 mmHg or more during sleep relative to the patient's awake baseline as assessed by PSG performed with a tracheostomy.

In some embodiments of any of the foregoing aspects, the method includes determining that the patient exhibits no intercostal retraction in a video recording of a respiratory sprinting trial.

In some embodiments of any of the foregoing aspects, the method includes determining that the patient exhibits no tachypnea in a video recording of a respiratory sprinting trial.

In some embodiments of any of the foregoing aspects, the method includes determining that the patient exhibits no respiratory paradox in a video recording of a respiratory sprinting trial.

In some embodiments of any of the foregoing aspects, the method includes determining that the patient exhibits no phase delay in a video recording of a respiratory sprinting trial.

In some embodiments of any of the foregoing aspects, the method includes determining that the patient exhibits an SpO₂ of less than 94% as assessed by a video recording of a respiratory sprinting trial.

In some embodiments of any of the foregoing aspects, the method includes determining that the patient exhibits an SpO₂ that does not differ by greater than 3% relative to the patient's awake baseline as assessed by a video recording of a respiratory sprinting trial.

In some embodiments of any of the foregoing aspects, the method includes determining that the patient exhibits a TCO₂ of greater than 45 mmHg as assessed by a video recording of a respiratory sprinting trial.

In some embodiments of any of the foregoing aspects, the method includes determining that the patient exhibits a TCO₂ that does not increase by 10 mmHg or more relative to the patient's awake baseline as assessed by a video recording of a respiratory sprinting trial.

In some embodiments of any of the foregoing aspects, the method further includes determining that the patient exhibits a respiration rate that is within age-adjusted norms as assessed by nocturnal respiration monitoring.

In some embodiments of any of the foregoing aspects, the method further includes determining that the patient exhibits no distress in a video recording of a respiratory sprinting trial.

In some embodiments of any of the foregoing aspects, the method further includes determining that the patient exhibits a respiration rate that is within age-adjusted norm as assessed by PSG performed with an open tracheostomy.

In another aspect, the disclosure provides a method of weaning a human patient that is on mechanical ventilation and that has XLMTM off of mechanical ventilation, wherein the patient has previously been administered a therapeutically effective amount of a viral vector including a transgene encoding MTM1, the method including: measuring in the patient one or more of (i) maximal inspiratory pressure on a ventilator, (ii) maximal expiratory pressure on a ventilator, (iii) positive end-expiratory pressure on a ventilator, (iv) SpO₂ level, (v) TcCO₂ level, (vi) petCO₂ level, and (vii) serum bicarbonate level of from about 22 mEq/L to about 27 mEq/L; and weaning the patient off of mechanical ventilation during daytime hours if the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, and (vii) a serum bicarbonate level of from about 22 mEq/L to about 27 mEq/L.

In some embodiments of the foregoing aspect, the method includes determining that the patient exhibits a maximal inspiratory pressure of about 50 cmH₂O or more on a ventilator.

In some embodiments of any of the foregoing aspects, the method includes determining that the patient exhibits a maximal expiratory pressure of about 40 cmH₂O or more on a ventilator.

In some embodiments of any of the foregoing aspects, the method includes determining that the patient exhibits a positive end-expiratory pressure of about 5 cmH₂O or less on a ventilator.

In some embodiments of any of the foregoing aspects, the method includes determining that the patient exhibits a SpO₂ of about 94% or more.

In some embodiments of any of the foregoing aspects, the method includes determining that the patient exhibits a TcCO₂ of from about 35 mmHg to about 45 mmHg.

In some embodiments of any of the foregoing aspects, the method includes determining that the patient exhibits a petCO₂ of from about 35 mmHg to about 45 mmHg.

In some embodiments of any of the foregoing aspects, the method includes determining that the patient exhibits a serum bicarbonate level of from about 22 mEq/L to about 27 mEq/L.

In some embodiments of any of the foregoing aspects, the method further includes determining that the patient exhibits vital signs and a weight that are within age-adjusted norms.

In some embodiments of any of the foregoing aspects, the method further includes determining that the patient exhibits a motor function score on the CHOP INTEND of greater than 45 or that neuromuscular development milestones have been met.

In another aspect, the disclosure provides a method of treating a human patient that has XLMTM and that is on mechanical ventilation, the method including: administering to the patient a therapeutically effective amount of a viral vector including a transgene encoding MTM1; determining that the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) an End petCO₂ of from about 35 mmHg to about 45 mmHg, and (vii) a serum bicarbonate level of from about 22 mEq/L to about 27 mEq/L; and weaning the patient off of mechanical ventilation during daytime hours.

In some embodiments of the foregoing aspect, the method includes determining that the patient exhibits a maximal inspiratory pressure of about 50 cmH₂O or more on a ventilator.

In some embodiments of any of the foregoing aspects, the method includes determining that the patient exhibits a maximal expiratory pressure of about 40 cmH₂O or more on a ventilator.

In some embodiments of any of the foregoing aspects, the method includes determining that the patient exhibits a positive end-expiratory pressure of about 5 cmH₂O or less on a ventilator.

In some embodiments of any of the foregoing aspects, the method includes determining that the patient exhibits a SpO₂ of about 94% or more.

In some embodiments of any of the foregoing aspects, the method includes determining that the patient exhibits a TcCO₂ of from about 35 mmHg to about 45 mmHg.

In some embodiments of any of the foregoing aspects, the method includes determining that the patient exhibits a petCO₂ of from about 35 mmHg to about 45 mmHg.

In some embodiments of any of the foregoing aspects, the method includes determining that the patient exhibits a serum bicarbonate level of from about 22 mEq/L to about 27 mEq/L.

In some embodiments of any of the foregoing aspects, the method further includes determining that the patient exhibits vital signs and a weight that are within age-adjusted norms.

In some embodiments of any of the foregoing aspects, the method further includes determining that the patient exhibits a motor function score on the CHOP INTEND of greater than 45 or that neuromuscular development milestones have been met.

In some embodiments of any of the foregoing aspects, the method further includes: determining that the patient exhibits one or more of (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an AHI of less than 5 events/hour as assessed by PSG performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with a tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TcCO₂ of greater than 45 mmHg as assessed by a video recording of a respiratory sprinting trial, and (xvi) a TcCO₂ that does not increase by 10 mmHg or more relative to the patient's awake baseline as assessed by a video recording of a respiratory sprinting trial; and continuing to wean the patient off of mechanical ventilation during daytime hours.

In some embodiments of any of the foregoing aspects, the method includes determining that the patient exhibits a TcCO₂ of from about 35 mmHg to about 45 mmHg as assessed by nocturnal respiration monitoring.

In some embodiments of any of the foregoing aspects, the method includes determining that the patient exhibits a petCO₂ of from about 35 mmHg to about 45 mmHg as assessed by nocturnal respiration monitoring.

In some embodiments of any of the foregoing aspects, the method includes determining that the patient exhibits an SpO₂ of about 94% or more as assessed by nocturnal respiration monitoring.

In some embodiments of any of the foregoing aspects, the method includes determining that the patient exhibits an AHI of less than 5 events/hour as assessed by PSG performed with an open tracheostomy.

In some embodiments of any of the foregoing aspects, the method includes determining that the patient exhibits a TcCO₂ of from about 35 mmHg to about 45 mmHg as assessed by PSG performed with an open tracheostomy.

In some embodiments of any of the foregoing aspects, the method includes determining that the patient exhibits a TcCO₂ that does not increase by 10 mmHg or more relative to the patient's awake baseline as assessed by PSG performed with an open tracheostomy.

In some embodiments of any of the foregoing aspects, the method includes determining that the patient exhibits a petCO₂ or a ptcCO₂ of less than 50 mmHg as assessed by PSG performed with an open tracheostomy.

In some embodiments of any of the foregoing aspects, the method includes determining that the patient exhibits a petCO₂ or ptcCO₂ that does not increase by 10 mmHg or more during sleep relative to the patient's awake baseline as assessed by PSG performed with a tracheostomy.

In some embodiments of any of the foregoing aspects, the method includes determining that the patient exhibits no intercostal retraction in a video recording of a respiratory sprinting trial.

In some embodiments of any of the foregoing aspects, the method includes determining that the patient exhibits no tachypnea in a video recording of a respiratory sprinting trial.

In some embodiments of any of the foregoing aspects, the method includes determining that the patient exhibits no respiratory paradox in a video recording of a respiratory sprinting trial.

In some embodiments of any of the foregoing aspects, the method includes determining that the patient exhibits no phase delay in a video recording of a respiratory sprinting trial.

In some embodiments of any of the foregoing aspects, the method includes determining that the patient exhibits an SpO₂ of less than 94% as assessed by a video recording of a respiratory sprinting trial.

In some embodiments of any of the foregoing aspects, the method includes determining that the patient exhibits an SpO₂ that does not differ by greater than 3% relative to the patient's awake baseline as assessed by a video recording of a respiratory sprinting trial.

In some embodiments of any of the foregoing aspects, the method includes determining that the patient exhibits a TcCO₂ of greater than 45 mmHg as assessed by a video recording of a respiratory sprinting trial.

In some embodiments of any of the foregoing aspects, the method includes determining that the patient exhibits a TcCO₂ that does not increase by 10 mmHg or more relative to the patient's awake baseline as assessed by a video recording of a respiratory sprinting trial.

In some embodiments of any of the foregoing aspects, the method further includes determining that the patient exhibits a respiration rate that is within age-adjusted norms as assessed by nocturnal respiration monitoring.

In some embodiments of any of the foregoing aspects, the method further includes determining that the patient exhibits no distress in a video recording of a respiratory sprinting trial.

In some embodiments of any of the foregoing aspects, the method further includes determining that the patient exhibits a respiration rate that is within age-adjusted norm as assessed by PSG performed with an open tracheostomy.

In another aspect, the disclosure provides a method of treating a human patient that has XLMTM and that is on mechanical ventilation, the method including: administering to the patient a therapeutically effective amount of a viral vector including a transgene encoding MTM1; measuring in the patient one or more of (i) maximal inspiratory pressure on a ventilator, (ii) maximal expiratory pressure on a ventilator, (iii) positive end-expiratory pressure on a ventilator, (iv) SpO₂ level, (v) TcCO₂ level, (vi) petCO₂ level, and (vii) serum bicarbonate level; and weaning the patient off of mechanical ventilation during daytime hours if that the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, and (vii) a serum bicarbonate level of from about 22 mEq/L to about 27 mEq/L.

In some embodiments of the foregoing aspect, the method includes determining that the patient exhibits a maximal inspiratory pressure of about 50 cmH₂O or more on a ventilator.

In some embodiments of any of the foregoing aspects, the method includes determining that the patient exhibits a maximal expiratory pressure of about 40 cmH₂O or more on a ventilator.

In some embodiments of any of the foregoing aspects, the method includes determining that the patient exhibits a positive end-expiratory pressure of about 5 cmH₂O or less on a ventilator.

In some embodiments of any of the foregoing aspects, the method includes determining that the patient exhibits a SpO₂ of about 94% or more.

In some embodiments of any of the foregoing aspects, the method includes determining that the patient exhibits a TcCO₂ of from about 35 mmHg to about 45 mmHg.

In some embodiments of any of the foregoing aspects, the method includes determining that the patient exhibits a petCO₂ of from about 35 mmHg to about 45 mmHg.

In some embodiments of any of the foregoing aspects, the method includes determining that the patient exhibits a serum bicarbonate level of from about 22 mEq/L to about 27 mEq/L.

In some embodiments of any of the foregoing aspects, the method further includes determining that the patient exhibits vital signs and a weight that are within age-adjusted norms.

In some embodiments of any of the foregoing aspects, the method further includes determining that the patient exhibits a motor function score on the CHOP INTEND of greater than 45 or that neuromuscular development milestones have been met.

In some embodiments of any of the foregoing aspects, the weaning off of mechanical ventilation includes a gradual reduction in ventilator support parameters including one or more of pressure, volume, and rate, followed by a progressive sprinting of off the ventilator, optionally wherein no more than one parameter of ventilator support is changed at a time.

In some embodiments of any of the foregoing aspects, upon administering the viral vector to the patient, the patient exhibits a change from baseline in hours of ventilation support over time, optionally wherein the patient exhibits the change from baseline in hours of ventilation support over time by about 24 weeks after administration of the viral vector to the patient.

In some embodiments of any of the foregoing aspects, upon administering the viral vector to the patient, the patient achieves functionally independent sitting for at least 30 seconds, optionally wherein the patient achieves the functionally independent slitting by about 24 weeks after administration of the viral vector to the patient.

In some embodiments of any of the foregoing aspects, upon administering the viral vector to the patient, the patient displays a reduction in required ventilator support to about 16 hours or less per day, optionally wherein the patient displays the reduction in required ventilator support by about 24 weeks after administration of the viral vector to the patient.

In some embodiments of any of the foregoing aspects, upon administering the viral vector to the patient, the patient displays a change from baseline on the CHOP INTEND, optionally wherein the patient displays the change from baseline on the CHOP INTEND by about 24 weeks after administration of the viral vector to the patient.

In some embodiments of any of the foregoing aspects, upon administering the viral vector to the patient, the patient displays a change from baseline in maximal inspiratory pressure, optionally wherein the patient displays the change from baseline in maximal inspiratory pressure by about 24 weeks after administration of the viral vector to the patient.

In some embodiments of any of the foregoing aspects, upon administering the viral vector to the patient, the patient displays a change from baseline in quantitative analysis of myotubularin expression in a muscle biopsy, optionally wherein the patient displays the change from baseline in quantitative analysis of myotubularin expression in a muscle biopsy by about 24 weeks after administration of the viral vector to the patient.

In some embodiments of any of the foregoing aspects, the transgene encoding MTM1 is operably linked to a muscle specific promoter.

In some embodiments of any of the foregoing aspects, the muscle specific promotor is a desmin promoter, a phosphoglycerate kinase (PGK) promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3 (PITX3).

In some embodiments of any of the foregoing aspects, the muscle specific promoter is a desmin promoter.

In some embodiments of any of the foregoing aspects, the viral vector is selected from the group consisting of adeno-associated virus (AAV), adenovirus, lentivirus, retrovirus, poxvirus, baculovirus, herpes simplex virus, vaccinia virus, and a synthetic virus.

In some embodiments of any of the foregoing aspects, the viral vector is an AAV.

In some embodiments of any of the foregoing aspects, the AAV is an AAV1, AAV2, AAV3, AAV4, AAV5, AAV6, AAV7, AAV8, AAV9, AAVrh10, or AAVrh74 serotype.

In some embodiments of any of the foregoing aspects, the viral vector is a pseudotyped AAV.

In some embodiments of any of the foregoing aspects, the pseudotyped AAV is AAV2/8.

In some embodiments of any of the foregoing aspects, the viral vector is resamirigene bilparvovec.

In another aspect, the disclosure provides a method of treating a human patient that has XLMTM and that is on mechanical ventilation, the method including: administering to the patient a therapeutically effective amount of an AAV2/8 viral vector including a transgene encoding MTM1 operably linked to a desmin promotor; determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met; weaning the patient off of mechanical ventilation during daytime hours; determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) no intercostal retraction in a video recording of a respiratory sprinting trial, (v) no tachypnea in a video recording of a respiratory sprinting trial, (vi) no respiratory paradox in a video recording of a respiratory sprinting trial, (vii) no phase delay in a video recording of a respiratory sprinting trial, (viii) an SpO₂ of less than 94% as assessed by a video recording of a respiratory sprinting trial, (ix) an SpO₂ that does not differ by greater than 3% relative to the patient's awake baseline as assessed by a video recording of a respiratory sprinting trial, (x) a TCO₂ of greater than 45 mmHg as assessed by a video recording of a respiratory sprinting trial, (xi) a TCO₂ that does not increase by 10 mmHg or more relative to the patient's awake baseline as assessed by a video recording of a respiratory sprinting trial, (xii) a respiration rate that is within age-adjusted norms as assessed by nocturnal respiration monitoring, (xiii) no distress in a video recording of a respiratory sprinting trial, and (xiv) a respiration rate that is within age-adjusted norm as assessed by PSG performed with an open tracheostomy; and continuing to wean the patient off of mechanical ventilation during daytime hours.

In another aspect, the disclosure provides a method of treating a human patient that has XLMTM and that is on mechanical ventilation, the method including: administering to the patient a therapeutically effective amount of an AAV2/8 viral vector including a transgene encoding MTM1 operably linked to a desmin promotor; determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met; weaning the patient off of mechanical ventilation during daytime hours; determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an AHI of less than 5 events/hour as assessed by PSG performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with an tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TCO₂ of greater than 45 mmHg as assessed by a video recording of a respiratory sprinting trial, (xvi) a TCO₂ that does not increase by 10 mmHg or more relative to the patient's awake baseline as assessed by a video recording of a respiratory sprinting trial; (xvii) a respiration rate that is within age-adjusted norms as assessed by nocturnal respiration monitoring, (xviii) no distress in a video recording of a respiratory sprinting trial, and (xix) a respiration rate that is within age-adjusted norm as assessed by PSG performed with an open tracheostomy; and continuing to wean the patient off of mechanical ventilation during daytime hours.

In another aspect, the disclosure provides a method of weaning a human patient that is on mechanical ventilation and that has XLMTM off of mechanical ventilation, wherein the patient has previously been administered a therapeutically effective amount of an AAV2/8 viral vector including a transgene encoding MTM1 operably linked to a desmin promotor, the method including: determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met; weaning the patient off of mechanical ventilation during daytime hours; determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) no intercostal retraction in a video recording of a respiratory sprinting trial, (v) no tachypnea in a video recording of a respiratory sprinting trial, (vi) no respiratory paradox in a video recording of a respiratory sprinting trial, (vii) no phase delay in a video recording of a respiratory sprinting trial, (viii) an SpO₂ of less than 94% as assessed by a video recording of a respiratory sprinting trial, (ix) an SpO₂ that does not differ by greater than 3% relative to the patient's awake baseline as assessed by a video recording of a respiratory sprinting trial, (x) a TCO₂ of greater than 45 mmHg as assessed by a video recording of a respiratory sprinting trial, (xi) a TCO₂ that does not increase by 10 mmHg or more relative to the patient's awake baseline as assessed by a video recording of a respiratory sprinting trial, (xii) a respiration rate that is within age-adjusted norms as assessed by nocturnal respiration monitoring, (xiii) no distress in a video recording of a respiratory sprinting trial, and (xiv) a respiration rate that is within age-adjusted norm as assessed by PSG performed with an open tracheostomy; and continuing to wean the patient off of mechanical ventilation during daytime hours.

In another aspect, the disclosure provides a method of weaning a human patient that is on mechanical ventilation and that has XLMTM off of mechanical ventilation, wherein the patient has previously been administered a therapeutically effective amount of an AAV2/8 viral vector including a transgene encoding MTM1 operably linked to a desmin promotor, the method including: determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met; weaning the patient off of mechanical ventilation during daytime hours; determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an AHI of less than 5 events/hour as assessed by PSG performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with an tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TcCO₂ of greater than 45 mmHg as assessed by a video recording of a respiratory sprinting trial, (xvi) a TcCO₂ that does not increase by 10 mmHg or more relative to the patient's awake baseline as assessed by a video recording of a respiratory sprinting trial; (xvii) a respiration rate that is within age-adjusted norms as assessed by nocturnal respiration monitoring, (xviii) no distress in a video recording of a respiratory sprinting trial, and (xix) a respiration rate that is within age-adjusted norm as assessed by PSG performed with an open tracheostomy; and continuing to wean the patient off of mechanical ventilation during daytime hours.

### BRIEF DESCRIPTION OF THE DRAWINGS

FIG. 1 is a schematic drawing of an exemplary pseudotyped adeno-associated virus (AAV) 2/8 (AAV2/8) viral vector for the expression of the human myotubularin 1 (hMTM1) gene. From left to right, the shaded arrows and rectangles represent the nucleic acid sequences encoding a human desmin (hDes) promotor (SEQ ID NO: 3) operatively linked to a Beta-globin Intron, a hMTM1 gene (SEQ ID NO: 4), a Beta-globin poly-adenylation signal (Beta-globin_pA), and flanking AAV2 inverted terminal repeat sequences (ITR). Abbreviations: AAV2 ITR, adeno associated virus 2 inverted terminal repeat; Beta-globin_pA, human Beta-globin polyadenylation signal; hDes, human desmin promotor; hMTM1, human myotubularin complementary DNA.

FIG. 2 is an illustration of the parameters recommended for evaluation of a patient by a physician of skill in the art prior to initiation of daytime weaning off of a mechanical ventilator.

FIG. 3 is a flowchart showing the stepwise criteria for determining if a patient is ready for the initiation of daytime, naptime, or nighttime weaning, respectively, and if a patient is ready for the continuation of daytime, naptime, or nighttime weaning, respectively, off of mechanical ventilation. Box with solid bold outline denotes the step criteria recommended for determining if a patient is ready for the initiation of daytime weaning off of a mechanical ventilator. Boxes with dashed bold outlines denote the step criterion recommended for determining if a patient is ready for the continuation of daytime weaning off of a mechanical ventilator. Abbreviations: CHOP INTEND, Children's Hospital of Philadelphia Infant Test of Neuromuscular Disorders; MIP, maximum inspiratory pressure; ORL, otorhinolaryngology; PEEP, positive end-expiratory pressure; PIP, peak inspiratory pressure; PSG, polysomnogram.

FIG. 4 is an illustration of the parameters recommended for evaluation of a patient by a physician of skill in the art prior to initiation of daytime weaning off of a mechanical ventilator.

### DEFINITIONS

As used herein, the term “about” refers to a value that is within 5% above or below the value being described. For example, “100 pounds” as used in the context of weight described herein includes quantities that are within 5% above or below 100 lbs. Additionally, when used in the context of a list of numerical quantities, it is to be understood that the term “about,” when preceding a list of numerical quantities, applies to each individual quantity recited in the list.

As used herein, the terms “administering,” “administration,” and the like refer to directly giving a patient a therapeutic agent (e.g., a pharmaceutical composition including a viral vector including a nucleic acid sequence encoding an Myotubularin 1 (MTM1) gene operably linked to a muscle specific promoter) by any effective route. Exemplary routes of administration are described herein and include systemic administration routes, such as intravenous injection, as well as routes of administration directly to the central nervous system of the patient, such as by way of intrathecal injection or intracerebroventricular injection, among others.

As used herein, the term “age-adjusted norms” refers to the process of a normalization of data by age, which is a technique that is used to allow populations of subjects to be compared when the age profiles of the populations are different.

As used herein, the term “awake baseline” refers to the basis of comparison acquired during the waking daytime hours of the patient and is defined at the time of weaning assessment with reassessment at subsequent trial encounters. As used herein, the term “waking daytime hours” refers to the hours from 7 am to 7 pm, such as 8 am, 9 am, 10 am, 11 am, 12 pm, 1 pm, 2 pm, 3 pm, 4 pm, 5 pm, or 6 pm. The duration of an awake baseline is not an absolute duration but will vary from patient-to-patient and by outcome. Furthermore, the duration of an “awake baseline trial” may vary by trial and may consist of the progressive adding of time (e.g., an awake baseline trial that is 15 minutes long in duration, an awake baseline trial that is 30 minutes long in duration, an awake baseline trial that is 45 minutes long in duration) in 15 minute increments. In some embodiments, the time during which an awake baseline trial is acquired is from 7 am to 7 pm, such as 8 am, 9 am, 10 am, 11 am, 12 pm, 1 pm, 2 pm, 3 pm, 4 pm, 5 pm, or 6 pm.

As used herein, the terms “apnea-hyponea index” or “AHI” refer to is the number of apneas or hypopneas recorded during a study per hour of sleep. As used herein, the term “apnea” refers to an event that is greater than or equal to 10 seconds during sleep in which a patient is observed to have an airflow reduction of greater than or equal to 90% from a baseline, wherein baseline is defined as the moderate of steady respiration and ventilation during the last 2 minutes prior to the event for patients with fixed respiration pattern, or the moderate of the 3 longest respirations during the last 2 minutes prior to the event for patients with variable respiration pattern and wherein airflow reduction is defined as occurring for greater than or equal to 90% of the apnea event. As used herein, the term “hyponea” refers to an event that is greater than or equal to 10 seconds during sleep in which a patient is observed to have a respiration rate reduction of greater than or equal to 50% from a baseline, wherein baseline is defined as the moderate of steady respiration and ventilation during the last 2 minutes prior to the event for patients with fixed respiration pattern, or the moderate of the 3 longest respirations during the last 2 minutes prior to the event for patients with a variable respiration pattern.

As used herein, the terms “Children's Hospital of Philadelphia Infant Test of Neuromuscular Disorders” or “CHOP INTEND” refer to a validated motor outcome measure developed for the evaluation of weak infants, such as those with a disease of skeletal muscle (e.g., X-linked myotubular myopathy (XLMTM)). CHOP INTEND uses a 0-64-point scale where higher scores indicate better motor function. As used herein, the term “motor function score” refers to a score on the 0-64-point scale of the CHOP INTEND (e.g., a scale of >45 on the CHOP INTEND).

As used herein, the term “distress” refers to the medical defined event of: any condition in which there exists an emotional or physical state of pain, sorrow, misery, suffering or discomfort.

As used herein, the term “dose” refers to the quantity of a therapeutic agent, such as a viral vector described herein, that is administered to a subject at a particular instant for the treatment of a disorder, such as to treat or ameliorate one or more symptoms of a neuromuscular disorder described herein (e.g., XLMTM). A therapeutic agent as described herein may be administered in a single dose or in multiple doses over the course of a treatment period, as defined herein. In each case, the therapeutic agent may be administered using one or more unit dosage forms of the therapeutic agent, a term that refers to a one or more discrete compositions containing a therapeutic agent that collectively constitute a single dose of the agent.

As used herein, the terms “effective amount,” “therapeutically effective amount,” and the like, when used in reference to a therapeutic composition, such as a vector construct described herein, refer to a quantity sufficient to, when administered to the subject, including a mammal, for example a human, effect beneficial or desired results, such as clinical results. For example, in the context of treating neuromuscular disorders, such as XLMTM, these terms refer to an amount of the composition sufficient to achieve a treatment response as compared to the response obtained without administration of the composition of interest. An “effective amount,” “therapeutically effective amount,” or the like, of a composition, such as a vector construct of the present disclosure, also include an amount that results in a beneficial or desired result in a subject as compared to a control.

As used herein, the terms “end tidal CO₂,” “ETCO₂,” and “petCO₂” refer to the volume of CO₂ entering the patient's lungs during inspiration (e.g., 35-45 mmHg of CO₂ are entering the patient's lungs during inspiration).

As used herein, the terms “maximal expiratory pressure” or “MEP” refer to a variable in mechanical ventilation including the strength of respiratory muscles obtained by having a patient exhale as strongly as possible against a mouthpiece; the maximum value is near total lung capacity.

As used herein, the term “intercostal retraction” refers to clinically-observable medical phenomenon that occurs when muscles between the ribs are pulled inward.

As used herein, the terms “maximal inspiratory pressure” or “MIP” refer to a variable in mechanical ventilation including the total airway pressure delivered, generally used to overcome both respiratory system compliance as well as airway resistance. In as pressure-controlled mode, the MIP includes the sum of the positive-end expiratory pressure and the “delta pressure.” As used herein, the term “delta pressure” refers to a variable in mechanical ventilation including the difference between the MIP and the positive-end expiratory pressure.

As used herein, the term “mechanical ventilation” refers to the medical term for artificial ventilation where mechanical means are used to assist or replace spontaneous breathing.

As used herein, the terms “monitored nocturnally,” “nocturnal monitoring,” “nocturnal respiration monitoring,” “respiration is monitored nocturnally” and the like refer to the monitoring of a patient during nighttime hours. As used herein, the term “nighttime hours” refers to the hours from 7 pm to 7 am, such as 8 pm, 9 pm, 10 pm, 11 pm, 12 am, 1 am, 2 am, 3 am, 4 am, 5 am, or 6 am. The duration of nocturnal monitoring is not an absolute duration but will vary from patient-to-patient and by outcome. Furthermore, the duration of nocturnal monitoring may vary by trial and may consist of the progressive reduction or addition of time (e.g., nocturnal monitoring that is 12 hours long in duration, nocturnal monitoring that is 11 hours long in duration, nocturnal monitoring that is 11 hours and 15 minutes long in duration) in 1 second increments.

As used herein, the term “neuromuscular development milestones” refers to behaviors or physical skills seen in infants and children as they grow and develop that include head control, sitting, voluntary grasp, ability to kick in supine, rolling, crawling or bottom shuffling, standing, and walking. The milestones are different for each age range e.g., sitting with support at hips is normal at 4 months of age, sitting with props is normal at 6 months of age, sitting stably is normal at 7-8 months of age, and sitting and pivoting is normal at 9 months of age, e.g., see De Sanctis, et al., Neuromuscular Disorders 26:754 (2016).

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the terms “oxygen saturation” or “SpO₂” refer to a measure of the level of hemoglobin that is bound to molecular oxygen in a patient. As used herein, the term “saturation of room air oxygen” refers to the amount of oxygen in a patient's bloodstream, as determined by the degree to which hemoglobin in the patient's red blood cells has bonded with oxygen molecules. Oxygen in the bloodstream is derived from the lungs and process of inhalation.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “phase delay” refers to the delay in time that includes the time when a mechanical ventilator first senses a trigger (e.g., pressure trigger) and the time when the ventilator responds by delivering gas flow. For example, in a transdiaphragmatic pressures (Pdi)-driven servoventilation system, ventilated pressure breaths are adjusted in response to either the patient's Pdi, such that triggering may occur when a patient initiates a breath, or a preset flow threshold, depending upon which is generated first. Subsequently, the ventilator algorithm generates a new flow signal that is offset from the patient's actual flow by 0.25 L/s and is delayed (e.g., delayed by 300 milliseconds), thereby allowing the signal to lag behind the patient's actual flow rate so that, once the patient initiates a breath, the sudden decrease in expiratory flow will initiate a ventilated breath.

As used herein, the terms “positive-end expiratory pressure” or “PEEP” refer to a variable in mechanical ventilation including the pressure maintained at the airways at the end of expiration (e.g., the pressure applied to the lungs never goes above 5 cmH₂O on the ventilator).

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, a therapeutic agent is considered to be “provided” to a patient if the patient is directly administered the therapeutic agent or if the patient is administered a substance that is processed or metabolized in vivo so as to yield the therapeutic agent endogenously. For example, a patient, such as a patient having a neuromuscular disorder described herein, may be provided a nucleic acid molecule encoding a therapeutic protein (e.g., MTM1) by direct administration of the nucleic acid molecule or by administration of a substance (e.g., viral vector or cell) that is processed in vivo so as to yield the desired nucleic acid molecule.

As used herein, the terms “respiration rate,” “respiratory rate,” or “RR” refer to the rate of breathing in a patient.

As used herein, the term “respiratory paradox” refers to a respiratory distress in a patient associated with damage to the structures involved in breathing whereby instead of moving outwardly when taking a breath, the chest wall or the abdominal wall of the patient moves inwardly.

As used herein, the terms “respiratory sprinting trial” or “sprinting” refer to a formal trials of spontaneous breathing to assess a patient who is likely to succeed or fail from liberation from mechanical ventilation. The duration of a respiratory sprinting trial is not an absolute duration but will vary from patient-to-patient and by outcome. Furthermore, “respiratory sprinting trials” may vary by time and may consist of the progressive adding of time (e.g., a respiratory sprinting trial that is 15 minutes long in duration, a respiratory sprinting trial that is 16 minutes long in duration, a respiratory sprinting trial that is 17 minutes long in duration) in single minute increments.

As used herein, the term “serum bicarbonate levels” refers to the level of CO₂ in a patient's blood (e.g., there are 22-27 mEq/L of CO₂ in the patient's blood).

As used herein, the terms “subject” and “patient” refer to an organism that receives treatment for a particular disease or condition as described herein (such as a neuromuscular disorder, e.g., XLMTM). Examples of subjects and patients include mammals, such as humans, receiving treatment for a disease or condition described herein.

As used herein, the term “tachyphena” refers to a condition of an abnormally rapid respiratory rate. As used herein, “tachyphena” is defined as a respiratory rate >60 breaths/minute in children that are less than 2 months of age, >50 breaths/minute in children that are 2 to 12 months of age, >40 breaths/minute in children that are between about 1 and about 5 years of age, and >20 breaths/minute in children that are greater than 5 years of ages.

As used herein, the terms “tracheostomy open” and “open tracheostomy” refer to a surgical procedure which consists of the making of an incision on the anterior aspect of a patient's neck and opening a direct airway through an incision in the trachea. The resulting stoma can serve independently as an airway or as a site for a tracheal tube or tracheostomy tube to be inserted; this tube allows a person to breathe without the use of the nose or mouth.

As used herein, the terms “transcutaneous CO₂” or “TcCO₂” refer to the level of CO₂ below a patient's skin.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “treat” or “treatment” refer to therapeutic treatment, in which the object is to prevent or slow down (lessen) an undesired physiological change or disorder, such as the progression of a neuromuscular disorder, such as XLMTM, among others. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. In the context of neuromuscular disorders, such as XLMTM, treatment of a patient may manifest in one or more detectable changes, such as an increase in the concentration of MTM1 protein or nucleic acids (e.g., DNA or RNA, such as mRNA) encoding MTM1, or an increase in MTM1 activity (e.g., by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or more. The concentration of MTM1 protein may be determined using protein detection assays known in the art, including ELISA assays described herein. The concentration of MTM1-encoding nucleic acids may be determined using nucleic acid detection assays (e.g., RNA Seq assays) described herein. Additionally, treatment of a patient suffering from a neuromuscular disorder, such as XLMTM, may manifest in improvements in a patient's muscle function (e.g., skeletal muscle function) as well as improvements in muscle coordination.

As used herein, the terms “X-linked myotubular myopathy” or “XLMTM” refer to the genetically-inherited neuromuscular disorder that is caused by mutations of the MTM1 gene and is characterized by symptoms including mild to profound muscle weakness, hypotonia (diminished muscle tone), feeding difficulties, and/or severe breathing complications. Human MTM1 has NCBI Gene ID NO 4534. An exemplary wild-type human MTM1 nucleic acid sequence is provided in NCBI RefSeq Acc. No. NM_000252.3 (SEQ ID NO: 1), and an exemplary wild-type myotubularin 1 amino acid sequence is provided in NCBI RefSeq Acc. No. NP 000243.1 (SEQ ID NO: 2).

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “vital signs” refers to a group of the four most important medical signs that indicate the status of the body's vital functions. The four main vital signs routinely monitored by medical professionals and health care providers comprise body temperature, pulse rate, respiration rate, and blood pressure.

As used herein, the term “weaning” refers to the discontinuation of mechanical ventilation including the gradual process of improving the load-to-capacity ratio of the mechanical and gas exchange capacity of the respiratory system to enable spontaneous and sustainable respiration. Weaning usually involves a gradual reduction in ventilator support (i.e., pressure, volume, and/or rate) for patients on higher settings and continuous support, followed by a progressive sprinting off the ventilator, whereby no more than one parameter of ventilator support is changed at the same time. As used herein, the term “daytime weaning” refers to weaning during waking daytime hours. The duration of daytime weaning is not an absolute duration but will vary from patient-to-patient and by outcome. Furthermore, “daytime weaning trials” may vary by time and may consist of the progressive adding of time (e.g., a daytime weaning trial that is 15 minutes long in duration, a daytime weaning trial that is 30 minutes long in duration, a daytime weaning trial that is 45 minutes long in duration) in 15 minute increments. As used herein, the term “naptime weaning” refers to weaning during waking daytime hours during which the patient has fallen asleep. The duration of naptime weaning is not an absolute duration but will vary from patient-to-patient and by outcome. Furthermore, “naptime weaning trials” may vary by time and may consist of the progressive adding of time (e.g., a naptime weaning trial that is 15 minutes long in duration, a naptime weaning trial that is 16 minutes long in duration, a naptime weaning trial that is 17 minutes long in duration) in 1 second increments. As used herein, the term “nighttime weaning” refers to weaning during nighttime hours. The duration of nighttime weaning is not an absolute duration but will vary from patient-to-patient and by outcome. Furthermore, “nighttime weaning trials” may vary by time and may consist of the progressive adding of time (e.g., a nighttime weaning trial that is 15 minutes long in duration, a nighttime weaning trial that is 16 minutes long in duration, a nighttime weaning trial that is 17 minutes long in duration) in 1 second increments.

### DETAILED DESCRIPTION

The present disclosure provides compositions and methods that can be used for treating neuromuscular disorders, particularly X linked myotubular myopathy (XLMTM). In accordance with the compositions and methods described herein, a patient (e.g., a human patient) having XLMTM may be administered a viral vector, such as an adeno-associated viral (AAV) vector, that contains a transgene encoding Myotubularin 1 (MTM1). The AAV vector may be, for example, a pseudotyped AAV vector, such as an AAV vector containing AAV2 inverted terminal repeats packaged within capsid proteins from AAV8 (AAV2/8). In some embodiments, the transgene is operably linked to a transcription regulatory element, such as a promoter that induces gene expression in a muscle cell. An exemplary promoter that may be used in conjunction with the compositions and methods of the disclosure is a desmin promoter.

The present disclosure is based, in part, on the discovery of an algorithm of parameters that enabled physicians of skill in the art to successfully discontinue mechanical ventilator support in children with XLMTM treated with gene therapy. Using the compositions and methods of the disclosure, an AAV vector may be administered to a patient in an amount that is sufficient to enhance a patient's expression of MTM1, a patient can then be assessed using the assessment parameters described herein for the readiness of the initiation of weaning off of a mechanical ventilator, a patient can then be weaned off of a mechanical ventilator, and a patient can then be further assessed using the assessment parameters described herein for the readiness of the continuation of weaning off of a mechanical ventilator.

In some embodiments, using the assessment parameters described herein, a patient is determined ready for the initiation of daytime weaning off a mechanical ventilator when a patient exhibits vital signs and a weight that are within the age-adjusted norms; a maximal inspiratory pressure (MIP) that is >−50 cmH₂O, a maximal expiratory pressure (MEP) that is >40 cmH₂O, and a positive end-expiratory pressure (PEEP) that is ≤5 cmH₂O on the ventilator; a saturation of room air oxygen (SpO₂) that is >94%, a transcutaneous CO₂ (TcCO₂) that is within 35-45 mmHg, an End Tidal CO₂ (ETCO₂) that is within 35-45 mmHg, and serum bicarbonate levels that are within 22-27 mEq/L; and a motor function score on the Children's Hospital of Philadelphia Infant Test of Neuromuscular Disorders (CHOP INTEND) that is >45 or that neuromuscular development milestones have been met.

In some embodiments, using the assessment parameters described herein, a patient is determined ready for the continuation of daytime weaning off a mechanical ventilator when a patient exhibits a TcCO₂ that is within 35-45 mmHg, an ETCO₂ that is within 35-45 mmHg, an SpO₂ that is >94%, and a respiration rate (RR) that is within the age-adjusted norms when respiration is monitored nocturnally; an apnea-hypopnea index (AHI) that is <5 events/hour, a TcCO₂ that is within 35-45 mmHg or that did not increases by 10 mmHg or greater above the awake baseline, an end-tidal CO₂ (petCO₂) or a partial pressure of CO₂ (ptcCO₂) that is <50 mmHg or that did not increase from the awake baseline by <10 during sleep, and a RR that is within the age-adjusted norm when a polysomnogram (PSG) is performed with the tracheostomy open; and when no distress, no intercostal retraction, no tachypnea, no respiratory paradox, no phase delay, an SpO₂ that is <94% or that does not differ by greater than 3% from baseline, and a TcCO₂ that is >45 mmHg or that did not increase by 10 mmHg or greater above the awake baseline is observed in the video recording of a respiratory sprinting trial.

The sections that follow provide a description of therapeutic agents and weaning assessment parameters that result in the determination that a patient is ready for the initiation or continuation of weaning off of a mechanical ventilator described above. The following sections also describe various transduction agents that may be used in conjunction with the compositions and methods of the disclosure.

### Methods of Treatment

### X-Linked Myotubular Myopathy

X-linked myotubular myopathy (XLMTM) is a rare, life-threatening, congenital myopathy caused by a loss-of-function mutation in the MTM1 gene and is characterized in most patients by profound muscle weakness and hypotonia at birth, which results in severe respiratory insufficiency, inability to sit up, stand or walk, and early mortality.

The myopathy associated with XLMTM impairs the development of motor skills such as sitting, standing, and walking. Affected infants may also have difficulties with feeding due to muscle weakness. Individuals with this condition often do not have the muscle strength to breathe on their own and must be supported with mechanical ventilation. Some affected individuals require mechanical ventilation only periodically, such as during sleep, while others require mechanical ventilation continuously. Patients having XLMTM may also have weakness in the muscles that control eye movement (ophthalmoplegia), weakness in other muscles of the face, and absent reflexes (areflexia).

In XLMTM, muscle weakness often disrupts normal bone development and can lead to fragile bones, an abnormal curvature of the spine (scoliosis), and joint deformities (contractures) of the hips and knees. Patients having XLMTM may have a large head with a narrow and elongated face and a high, arched roof of the mouth (palate). Patients may also have liver disease, recurrent ear and respiratory infections, or seizures.

As a consequence of their severe breathing difficulties, patients having XLMTM usually survive only into early childhood; however, some patients with this condition have lived into adulthood. The compositions and methods of the disclosure provide the important medical benefit of being able to prolong the lifetimes of such patients by restoring functional MTM1 expression. Moreover, the compositions and methods described herein can be used to improve patients' quality of life post-treatment, as the disclosure provides a series of guidelines that can be used to determine a patient's eligibility for being weaned off of mechanical ventilation.

### Vectors for Delivery of Exogenous Nucleic Acids to Target Cells

### Viral Vectors for Nucleic Acid Delivery

Viral genomes provide a rich source of vectors that can be used for the efficient delivery of a gene of interest (e.g., a transgene encoding MTM1) into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the invention are recombinant nucleic acid constructs that include (1) a transgene to be expressed (e.g., a polynucleotide encoding a MTM1 protein) and (2) viral nucleic acids that facilitate integration and expression of the heterologous genes. The viral nucleic acids may include those sequences of AAV that are required in cis for replication and packaging (e.g., functional inverted terminal repeats (ITRs)) of the DNA into a virion. In typical applications, the transgene encodes MTM1, which is useful for correcting a MTM1 mutation in patients suffering from neuromuscular disorders, such as XLMTM. Such rAAV vectors may also contain marker or reporter genes. Useful rAAV vectors have one or more of the AAV wild type genes deleted in whole or in part, but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery. The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of MTM1 in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Resamirigene Bilparvovec

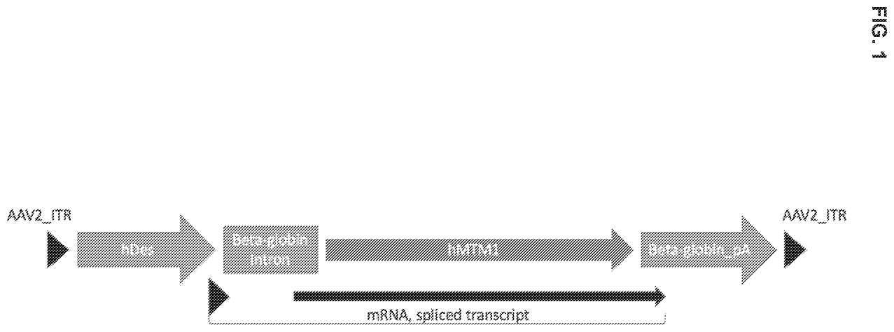

As described herein, a pseudotyped AAV vector including a nucleic acid sequence encoding a MTM1 gene (SEQ ID NO: 4) operably linked to a desmin promotor (SEQ ID NO:3; FIG. 1) flanked by AAV2 ITR and packaged within capsid proteins from AAV8 (AAV2/8) as well as the other genetic components listed in Table 1, refers to the compound known by the international nonproprietary name (INN) of resamirigene bilparvovec.

In some embodiments, a method of treating a disorder (e.g., XLMTM) or alleviating one or more symptoms of a disorder (e.g., XLMTM) in a human patient in need thereof, includes administering to the patient a therapeutically effective amount of resamirigene bilparvovec during a treatment period.

In some embodiments, a method of weaning a human patient off of mechanical ventilation, includes a patient that has previously been administered a therapeutically effective amount of resamirigene bilparvovec.

TABLE 1
Resamirigene Bilparvovec Nucleic Acid Sequence (SEQ ID NO: 5)

| Range | | |
|---|---|---|
| (nucleotides, | | |
| relative to | Length | |
| SEQ ID NO: 5) | (nucleotides) | Genetic Component |
| 3080-3198 | 119 | AAV2 ITR |
| 3199-3256 | 58 | Linker sequence |
| 3257-4316 | 1,060 | Human desmin promoter (SEQ ID NO: 3) |
| 4317-4354 | 38 | Linker sequence |
| 4355-4460 | 106 | Human Beta-globin intron |
| 4373-4848 | 476 | Human Beta-globin intron |
| 4458-4902 | 445 | Human Beta-globin intron |
| 4917-6738 | 1,822 | Human MTM1 coding sequence (SEQ ID |
| | | NO: 4) |
| 6739-6759 | 21 | Linker sequence |
| 6760-7519 | 760 | Human Beta-globin poly-adenylation |
| | | sequence |
| 7520-7551 | 32 | Linker sequence |
| 7552 | 7,696 | AAV2 ITR |

As described herein, resamirigene bilparvovec refers to the AAV vector having the nucleic acid sequence of SEQ ID NO: 5, shown below:

TCGCGCGTTT CGGTGATGAC GGTGAAAACC TCTGACACAT GCAGCTCCCG 50
GACGTCATTG TCGATCCTGC AGGCGTACGG TAAAAAAAGG CATAGCTAAC 100
AAGGTGTGGA AAAAGAATTA GTGGTTAGAG AGTGAGCTAT TCGTTGAAAC 150
AATTGCGTTC TTGAAACAAT TCTTGCTGGT AAAATGTCAC ATTTTATGTG 200
ACTACAGGTG GAGGATTGGC ACATAACCTA ACCAGTGGGG GAAACAATTG 250
ACCTCTGGAT TTGTCCAAGT GTATAGTAGC ATTTGCCCAA TCGAATGGTC 300
CTGGTAAGGT GTTAATGTTG ACTAGAACCA AAGGTGGAAG TTGCAGGGAA 350
ACTGGTTTAG TACAAGGGTG GACACCAGGC AGTCATCCAG AGGCCCATTA 400
AAGGCCTTGG AATGTTTTTC CGAAGGAGAA TCACTCCCTC TTCTCTCGCT 450
TAAAGTTTTA GGGGATTCAT GAACAGCTGC TGTGGGATAG TTTCATGTCC 500
CTAGCAATTG TAAAGCAACT GAGGGTGGCT TAAACCAGTT TTAGCTTTAG 550
GGTTAGGGTT ACTGGACTAA AATTTGAGAA ATTCATAAAT CTTAAGGAAA 600
TCCATTGTGA GTTTTCATTA TGAGTGCATC CAATGTATAA TTTCCATGAC 650
CCTCCCATGC AAGTGAGCAT GTGAATCAGG AAACGTTACA AGAACCCAAC 700
AAACTCAACC ACTACTAGAC AGGCGATCAC TTCCAGTTAG TATGCAACTI 750
TCTGTGTAAT TTTAGTTACC ATTAAAATCT GGATGACCTT AGTGTAAGGA 800
AAAAATACCT TGAATAGTGT TAAAGATGTA CACTTGGTGT CAGGCATTGT 850
AACATTGATA AATCTGTGTA AGGTGCTTTT TGAAAACTTC AAAGCTGCAT 900
CAAGTCAAGT ACAAGAAAGG CCATGGCTGC TAAAGCTGTT GAAGATGTGG 950
GATGGAACTG GGTCACATTG GTGTTAACAG CGTTGTGCAG AGCCGGCAGG 1000
ATCTTGGTGT GAGCGAACAT TAGTCTATTT AATAAAGCTG TGTGAATGTT 1050
GTAGAGGTGA GGATGCTCAC TTGAAAACTC ACTGAAGAAC ACTTGGCCCC 1100
TTGAACTAAA GTGCTTCTAT CAAGTTCAGT GAGAAATTCC GAATTACAAG 1150
CATAGGTACT AGAAAAGTTT TGAAAAGCAG TATAGAGCAA CATAAGCACA 1200
TTCATAAAAT TAGTGATGTA GAAAGTGAAA TTTCCACGTA TGGTCACTCC 1250
CAGAGAAAAA AAATACGTTT ATTTACCTTT TTTAAAAATA GGGGATTTCA 1300
GGCCGGGTGA GGTGGCTCAC GCCTGTAATC CCAGCACTTT GGGAGGCCCA 1350
GGTGGGCGGA TCACCTGAGG TCAGGAGTTG GAGGGATGGC AAATCCCATC 1400
TCTACAAAAT ATACAAAAAA ATAGCTGGGT GTGTTGGCAG GCGCCTGTAA 1450
TCCCAGCTAC TCGGAAGGCT GAGGCAGGAG AATCCCTGGA ACCAGGGATG 1500
TGGAGGTTGC AGTGAGCCGA GATTGTGTAA CTGCATTCCA GCCTGGGCAA 1550
CAAGAGCAAG ACTCCGTATC AGGAAAAAAA AAAGGGGGGG TTGGATTTCG 1600
CTTGTTGCAT AGGTTGGTCT CAAACTCCTG GCCTCAAGTG ATTCTCCTGC 1650
CTCTGCCTCC CAAAGTGCTG AGATTACAGG TGTGAGGCAC CATGCCAGGT 1700
CTCTTACTGT TTGTAATTAA ATACATACAC ATTTTGTGTG TTTGTGTGCA 1750
CCTTTATAAA GTCAAAGGTG ATAGTAACCC ATTTAAGTTC CTACTCAATT 1800
TTACTTTCCA GGGATAACTA ACTACTTTTT CTTTTTGAGA TGGAGTCTCG 1850
CTGTGTAGCC CAGGCTGGAG TGCAGTGGCA CCATCTCGGC TCACTGCAAG 1900
CTCCTCCTCC CTGGTTCACG CTATTCTCCT GCCTCAGCCT CCCCAACAAC 1950
TAGGACTACA GGCTCACCTC GCCATACCTG GCTAATTTTT TGTATTTTTA 2000
GTAGAGACAG GGTTTCACTG TGTTAGCCAG GATGGTCTCG ATCTCCTGAC 2050
CTTGTGATCC GCCTGCCTCT GCCTCCCAAA GTGCTGGGAT TACAGGCATG 2100
AGCAACCTCA CCCAGCTGGG ATAACTACTT TTTACAGGTT GATATTCTTT 2150
TGGACTTTTC CCCTGTGTAA AAATATACTA TATTTGTTAT GTACATATTA 2200
TGTACATACA GACACAAATT GGACCATTCT CAGTATAATG ATTCTCAGGT 2250
TTTTTTTTTT TTTTTGAGGT GGGGAACTAG ATAATTATGG ACATCTTTCC 2300
ATACTAGCAT ATCAATATCT ACCTCATTCT TTTTAATATT TTTGCTAGTA 2350
TTCCATTGTA TGAATGTCCT ATGATTTACT TAACCTGTCC ATCAATATTT 2400
GTTTCCAGGT TTTTGCTATT ATAATGCTGC TGCAAAGTAC ATCCTCACAC 2450
ATCTTTATTT TGTCTATTCA TATTTCTGTA AGATAGGTTA CTAAAGTTGG 2500
AACTGCCAAA TTAACACTAT CATACTATTT TGTTTTTTAA TTTTAATTTT 2550
TTAAAAAATG TAAAATGTGC AATTTCAAGA GGAGAAACTT GAACACAAGG 2600
AGCAAAATCT ATTTTTATAA CATCCTATTA AAAGCTTGCT TTACATAAAG 2650
ATTTTGAAAG AATAGCATAA ATACAAGATT TCTATTTTAA TTGGATTCTT 2700
AGGGCTAATA AAATAATCAG CCTTAGCACT TATTTATTTA TTTTTTTTGA 2750
GAGGGAGTCT CGCTCTGTTG TCCATGCTGG AGTGCAGTGG CGTGATCTCG 2800
GCTCACTGCA AGCTCCACCT CATGAGTTCA CACCATTCTC CTGCCTCAGT 2850
CTCCCGAGTA GCTGGGACTC CAGGCGCCCT CTACAAAGCC CGTCTAATTT 2900
TTTTTGTATT TTTAGTAGAG ACAGGGTTTC ACTGTGTTAG CCAGGATGGT 2950
CTTGATCTCC TGACCTTGTG ATCTGCCCGC CTCGGCCTCC CAAAGTGCTG 3000
GGATTATAGG CTTGAGCCAC TGCTCCCGGC CAGCACTTAT TTTTATAATT 3050
CTTCATGATT ACTGTGTTAC TGTCCCATGG GCCGCCAGGG CCAGCTAGGT 3100
TGGCCACTCC CTCTCTGCGC GCTCGCTCGC TCACTGAGGC CGGGCGACCA 3150
AAGGTCGCCC GACGCCCGGG CTTTGCCCGG GCGGCCTCAG TGAGCGAGCG 3200
AGCGCGCAGA GAGGGAGTGG CCAACTCCAT CACTAGGGGT TCCTCCTAGC 3250
ACGCGCTACC CCCTGCCCCC CACAGCTCCT CTCCTGTGCC TTGTTTCCCA 3300
GCCATGCGTT CTCCTCTATA AATACCCGCT CTGGTATTTG GGGTTGGCAG 3350
CTGTTGCTGC CAGGGAGATG GTTGGGTTGA CATGCGGCTC CTGACAAAAC 3400
ACAAACCCCT GGTGTGTGTG GGCGTGGGTG GTGTGAGTAG GGGGATGAAT 3450
CAGGGAGGGG GCGGGGGACC CAGGGGGCAG GAGCCACACA AAGTCTGTGC 3500
GGGGGTGGGA GCGCACATAG CAATTGGAAA CTGAAAGCTT ATCAGACCCT 3550
TTCTGGAAAT CAGCCCACTG TTTATAAACT TGAGGCCCCA CCCTCGACAG 3600
TACCGGGGAG GAAGAGGGCC TGCACTAGTC CAGAGGGAAA CTGAGGCTCA 3650
GGGCCAGCTC GCCCATAGAC ATACATGGCA GGCAGGCTTT GGCCAGGATC 3700
CCTCCGCCTG CCAGGCGTCT CCCTGCCCTC CCTTCCTGCC TAGAGACCCC 3750
CACCCTCAAG CCTGGCTGGT CTTTGCCTGA GACCCAAACC TCTTCGACTT 3800
CAAGAGAATA TTTAGGAACA AGGTGGTTTA GGGCCTTTCC TGGGAACAGG 3850
CCTTGACCCT TTAAGAAATG ACCCAAAGTC TCTCCTTGAC CAAAAAGGGG 3900
ACCCTCAAAC TAAAGGGAAG CCTCTCTTCT GCTGTCTCCC CTGACCCCAC 3950
TCCCCCCCAC CCCAGGACGA GGAGATAACC AGGGCTGAAA GAGGCCCGCC 4000
TGGGGGCTGC AGACATGCTT GCTGCCTGCC CTGGCGAAGG ATTGGTAGGC 4050
TTGCCCGTCA CAGGACCCCC GCTGGCTGAC TCAGGGGCGC AGGCCTCTTG 4100
CGGGGGAGCT GGCCTCCCCG CCCCCACGGC CACGGGCCGC CCTTTCCTGG 4150
CAGGACAGCG GGATCTTGCA GCTGTCAGGG GAGGGGAGGC GGGGGCTGAT 4200
GTCAGGAGGG ATACAAATAG TGCCGACGGC TGGGGGCCCT GTCTCCCCTC 4250
GCCGCATCCA CTCTCCGGCC GGCCGCCTGC CCGCCGCCTC CTCCGTGCGC 4300
CCGCCAGCCT CGCCCGGACT CTAGAGGATC CAGATCTAAG CTTCTCTGGT 4350
CACCGATCCT GAGAACTTCA GGGTGAGTCT ATGGGACCCT TGATGTTTTC 4400
TTTCCCCTTC TTTTCTATGG TTAAGTTCAT GTCATAGGAA GGGGAGAAGT 4450
AACAGGGTAC ACATATTGAC CAAATCAGGG TAATTTTGCA TTTGTAATTT 4500
TAAAAAATGC TTTCTTCTTT TAATATACTT TTTTGTTTAT CTTATTTCTA 4550
ATACTTTCCC TAATCTCTTT CTTTCAGGGC AATAATGATA CAATGTATCA 4600
TGCCTCTTTG CACCATTCTA AAGAATAACA GTGATAATTT CTGGGTTAAG 4650
GCAATAGCAA TATTTCTGCA TATAAATATT TCTGCATATA AATTGTAACT 4700
GATGTAAGAG GTTTCATATT GCTAATAGCA GCTACAATCC AGCTACCATT 4750
CTGCTTTTAT TTTATGGTTG GGATAAGGCT GGATTATTCT GAGTCCAAGC 4800
TAGGCCCTTT TGCTAATCAT GTTCATACCT CTTATCTTCC TCCCACAGCT 4850
CCTGGGCAAC GTGCTGGTCT GTGTGCTGGC CCATCACTTT GGCAAAGAAT 4900
TCCGCGGGCG GCCGCAAGTT TCCAGGATGG CTTCTGCATC AACTTCTAAA 4950
TATAATTCAC ACTCCTTGGA GAATGAGTCT ATTAAGAGGA CGTCTCGAGA 5000
TGGAGTCAAT CGAGATCTCA CTGAGGCTGT TCCTCGACTT CCAGGAGAAA 5050
CACTAATCAC TGACAAAGAA GTTATTTACA TATGTCCTTT CAATGGCCCC 5100
ATTAAGGGAA GAGTTTACAT CACAAATTAT CGTCTTTATT TAAGAAGTTT 5150
GGAAACGGAT TCTTCTCTAA TACTTGATGT TCCTCTGGGT GTGATCTCGA 5200
GAATTGAAAA AATGGGAGGC GCGACAAGTA GAGGAGAAAA TTCCTATGGT 5250
CTAGATATTA CTTGTAAAGA CATGAGAAAC CTGAGGTTCG CTTTGAAACA 5300
GGAAGGCCAC AGCAGAAGAG ATATGTTTGA GATCCTCACG AGATACGCGT 5350
TTCCCCTGGC TCACAGTCTG CCATTATTTG CATTTTTAAA TGAAGAAAAG 5400
TTTAACGTGG ATGGATGGAC AGTTTACAAT CCAGTGGAAG AATACAGGAG 5450
GCAGGGCTTG CCCAATCACC ATTGGAGAAT AACTTTTATT AATAAGTGCT 5500
ATGAGCTCTG TGACACTTAC CCTGCTCTTT TGGTGGTTCC GTATCGTGCC 5550
TCAGATGATG ACCTCCGGAG AGTTGCAACT TTTAGGTCCC GAAATCGAAT 5600
TCCAGTGCTG TCATGGATTC ATCCAGAAAA TAAGACGGTC ATTGTGCGTT 5650
GCAGTCAGCC TCTTGTCGGT ATGAGTGGGA AACGAAATAA AGATGATGAG 5700
AAATATCTCG ATGTTATCAG GGAGACTAAT AAACAAATTT CTAAACTCAC 5750
CATTTATGAT GCAAGACCCA GCGTAAATGC AGTGGCCAAC AAGGCAACAG 5800
GAGGAGGATA TGAAAGTGAT GATGCATATC ATAACGCCGA ACTTTTCTTC 5850
TTAGACATTC ATAATATTCA TGTTATGCGG GAATCTTTAA AAAAAGTGAA 5900
GGACATTGTT TATCCTAATG TAGAAGAATC TCATTGGTTG TCCAGTTTGG 5950
AGTCTACTCA TTGGTTAGAA CATATCAAGC TCGTTTTGAC AGGAGCCATT 6000
CAAGTAGCAG ACAAAGTTTC TTCAGGGAAG AGTTCAGTGC TTGTGCATTG 6050
CAGTGACGGA TGGGACAGGA CTGCTCAGCT GACATCCTTG GCCATGCTGA 6100
TGTTGGATAG CTTCTATAGG AGCATTGAAG GGTTCGAAAT ACTGGTACAA 6150
AAAGAATGGA TAAGTTTTGG ACATAAATTT GCATCTCGAA TAGGTCATGG 6200
TGATAAAAAC CACACCGATG CTGACCGTTC TCCTATTTTT CTCCAGTTTA 6250
TTGATTGTGT GTGGCAAATG TCAAAACAGT TCCCTACAGC TTTTGAATTC 6300
AATGAACAAT TTTTGATTAT AATTTTGGAT CATCTGTATA GTTGCCGATT 6350
TGGTACTTTC TTATTCAACT GTGAATCTGC TCGAGAAAGA CAGAAGGTTA 6400
CAGAAAGGAC TGTTTCTTTA TGGTCACTGA TAAACAGTAA TAAAGAAAAA 6450
TTCAAAAACC CCTTCTATAC TAAAGAAATC AATCGAGTTT TATATCCAGT 6500
TGCCAGTATG CGTCACTTGG AACTCTGGGT GAATTACTAC ATTAGATGGA 6550
ACCCCAGGAT CAAGCAACAA CAGCCGAATC CAGTGGAGCA GCGTTACATG 6600
GAGCTCTTAG CCTTACGCGA CGAATACATA AAGCGGCTTG AGGAACTGCA 6650
GCTCGCCAAC TCTGCCAAGC TTTCTGATCC CCCAACTTCA CCTTCCAGTC 6700
CTTCGCAAAT GATGCCCCAT GTGCAAACTC ACTTCTGACC GGTCCGAGGG 6750
CCCAGATCTA ATTCACCCCA CCAGTGCAGG CTGCCTATCA GAAAGTGGTG 6800
GCTGGTGTGG CTAATGCCCT GGCCCACAAG TATCACTAAG CTCGCTTTCT 6850
TGCTGTCCAA TTTCTATTAA AGGTTCCTTT GTTCCCTAAG TCCAACTACT 6900
AAACTGGGGG ATATTATGAA GGGCCTTGAG CATCTGGATT CTGCCTAATA 6950
AAAAACATTT ATTTTCATTG CAATGATGTA TTTAAATTAT TTCTGAATAT 7000
TTTACTAAAA AGGGAATGTG GGAGGTCAGT GCATTTAAAA CATAAAGAAA 7050
TGAAGAGCTA GTTCAAACCT TGGGAAAATA CACTATATCT TAAACTCCAT 7100
GAAAGAAGGT GAGGCTGCAA ACAGCTAATG CACATTGGCA ACAGCCCCTG 7150
ATGCCTATGC CTTATTCATC CCTCAGAAAA GGATTCAAGT AGAGGCTTGA 7200
TTTGGAGGTT AAAGTTTTGC TATGCTGTAT TTTACATTAC TTATTGTTTT 7250
AGCTGTCCTC ATGAATGTCT TTTCACTACC CATTTGCTTA TCCTGCATCT 7300
CTCAGCCTTG ACTCCACTCA GTTCTCTTGC TTAGAGATAC CACCTTTCCC 7350
CTGAAGTGTT CCTTCCATGT TTTACGGCGA GATGGTTTCT CCTCGCCTGG 7400
CCACTCAGCC TTAGTTGTCT CTGTTGTCTT ATAGAGGTCT ACTTGAAGAA 7450
GGAAAAACAG GGGGCATGGT TTGACTGTCC TGTGAGCCCT TCTTCCCTGC 7500
CTCCCCCACT CACAGTGACC GGCCGCTCTA GGAGGAACCC CTAGTGATGG 7550
AGTTGGCCAC TCCCTCTCTG CGCGCTCGCT CGCTCACTGA GGCCGGGCGA 7600
CCAAAGGTCG CCCGACGCCC GGGCTTTGCC CGGGCGGCCT CAGTGAGCGA 7650
GCGAGCGCGC AGAGAGGGAG TGGCCAACCT AGAGGCCGCC AGGGCCATAT 7700
TTCTCAATTT TTAAATTTTT CAAAAAAATT AATCCTTAAT GTGCATATTT 7750
TTGAATTGTT AATATAACTT TTTGAGGTGA TGTCTTCATG TGTTTCAACT 7800
ACTTAAAAAC TTTTAAACAG TATATAATAA AAAATCTTCC AGGCCACTCA 7850
CACCTGTAAT CCCAGCACTT TGGGAGGCTG AGGTGGGCAG ATCACCTGAG 7900
GGCAGGAGTT CGAGACCAGC CTGGCCAATA TATATATATT CATATATTCA 7950
TATATATATA TATATTCATA TATTCATATA TATATATTCA TATATTCATA 8000
TATATATATA TATATATATA TAGCAAAACC TCATCTCTAA TAAAATACAA 8050
AAATTAGCTG AGCGTGGTGA TGGATGCCTG TAGTCCCAGC TACTCGGGAG 8100
GCTGAGGCAG GAGAATCTCT TGAACCTGGG AGGTGGAGGT TGCAGTGAGC 8150
TGAGATGGTG CCACTGCCCT CCAGCCTGAG TGACAGAGCG AGACTCGGTC 8200
TCCAAAAAAA AACAACAAAA AAATCTTCCA TCCTTGTCTC CCATCCACCC 8250
CTTCCCCCCA GCATGTACTT GCAGACTTTA TGCATATACA GTGAGTACTG 8300
TATATACACA AATAATAAAA AAATCATATA TATAATATAT GTAATTCCCC 8350
TTTACATGAA AGGTAGCACA CTGGTCTGTA CAGTCTGTCT GCACTGTGCT 8400
ATTTCACTTT ATATTTTTAT AGTTTGACAG AGTTCTAACA TTTCTTTTTT 8450
TTTTTTTTTA ACAGAGTCTT GTTCCTGATT GTTAAATTTT AAAGCATCCT 8500
AAAGTTTGGT TTCACACTTG AATGAATACC ATGTAAGGAT TCACTTACAT 8550
AGATGTGGTT GCCTGAATCT TAAGAATAAA ATAACATTGT TTGTATTTAT 8600
TTAAATTAGT GTTCCTTTTA TGGTTTGCCT GAAAGCACAA CAAAATCCTC 8650
ACCAAGATAT TACAATTATG ACTCCCATAC AGGTAAACTG TTTAGAGATT 8700
GGCAAGCACC TTTTAATGAA AGGAGTCAGC CAGCTTAGTG TGCAGTATTT 8750
ATTTCTGCCG GAAGAGGGAG CTTCAGGGAC AGACTTTGGT TTAGTCATGA 8800
AGCCTCCAGC ACTCCCAAGC GGTTGTGGTT GACCAAGCAA TTTATGCTTT 8850
TACCTTTCTA CTTCCAGAGG CTTGTTTACT TATCAGTAAG CATTAATTTA 8900
GTGTCCCCTC AGATGCCTTT TACTTTCTTC TTTTCTGCCT AGAATAAGCT 8950
GCTCTTCCAA TTTTGCAGCT ACATGTTTCC ACCCCAGTTG GAATTTCTCC 9000
ATAACATCCA TTGTAGCTAT CCTTCAATCT ACAGCCTCTA TTTCCTGTTA 9050
TAGCTGGTCA GGTCTAATCC CTCAAAATAC TCTGTCCCCT GCTTCCCTTA 9100
TCTGCTGGCC ACCTTTTTCC CCCACATACA CACTGCCATG TCCCACCCTT 9150
CACTCAAGTT GTTCCCTGCC ACCTCAACAA ATTTAAGTCC ATAAAATAGA 9200
GTAAGTGTTC CTGACTGTTA AATTTTAAAG CATCCCAAAG TCTGATTTCA 9250
CACTCGAATG AATACTATGT ACGGATTCAT TTACATAGAT GCGGTTGCAT 9300
GAGTCTTAAC AAAAAAATAA CATTATTTGT ATTTATTCAA AGTACTGTCA 9350
AGATATAATG TCAAGACCTA ATTCAAAGGT TCCACAAAGC CTTCCTTGAC 9400
TGCCCCCAAC GAAGATTATC CATTTTCCCT GAAATCCCAT TGACTTTTCT 9450
ATTTTGTAAG GAGGCTCGTG AGACTCTGTC TAAAAACAAA ACAAAACAAA 9500
AAGAAACAAT CAAACGGCTT GCTTCTGTTC TTTGATCTGC TAGTAAGCAA 9550
AAATTACACA TGGTGACAGG AGCTATGTGA GGCTGTCAGG TTGAATGGGA 9600
GGAGTTTGGG ATCCTGCTTG TGGATGGTTG GAAGAGGCTT TCGGGAAAGA 9650
CAGTATTTAT GTGAGACCTG GAAGATGGGC CTTAGCTTTG CAGAAGGTGG 9700
AGAGGCAGGA AATAGCACGG GGGCCCTGGG GCTGGAAGAC TTGGGCATAT 9750
TTGAGGAACA GAAAGGAGAC CAGCATAACT GAGGTGGGAA AAGCATGTGA 9800
AGAGATGGGG CTGGAGGAGG CCGGGAGTGG TGGCTCACGC CTGTAATCCC 9850
AGCACTTTGG GAGGCCAAGG CAGGCGGATC ATGAGCTCAG GAGATTGAGA 9900
CCATCCTGGC TAACACGGTG AAACCCCCTC TCTACTAAAA ATACAAAAAA 9950
AAAAAAAAAA AAAATTAGCT GGGCGTGGTG GCAGGAGCCT GTAGTCCCAG 10000
CTACCTGGGA GGCTGAGGCA GGAGAATGGC GTGAACCTGG AAGGCTGAGC 10050
TTGCAGTGAG CCGAGATTGC ACCACTGCAC TCCAGCCTGG GAGACAGAGA 10100
GAGACTCCCT CTCAAAAAAA CAAACAAACG AAACAAAACA AAACAAAAAT 10150
TAGCCAGGCG TGGTGGTATG CACCTGTAAT CCCAGCTACT CGGGAGGTTG 10200
AGGCAGGAGA AACGCTTGAA CTCAGGAGGC GGAGGTTGCA GTGAGCCGAG 10250
ACTGCGCCAC TGCACTCCAG CCTGGGTGAC AGAGGGAGAC TCCATCTCAA 10300
AAAAAAAAAT TTTTTTTTTT TTACAAACGG TGTCTCCCTC TGTCGCCCAG 10350
GCTGGAGTGC AGTGGTGTGA TCACAGCTCA CTCCAGCCTC AACCTCCCCA 10400
GCTGAAGCCA TCCTCTTGCC TCAGCCTCCT AAGTAGCTGG GACTACAGGC 10450
GCGCACCTCC AGGCTTGGCT CTTATTCTTT TTATTGTTTT TGAAACTATA 10500
GAACCTATTT TTAAAAAATG TTTTGGTTGT TTTTATTGCT GCTTTTCCTT 10550
TTGGGGTTAG AACACAAGTT TTGATGGGAA ACAGGTTAGA ACACATTCAT 10600
CTCTTCCCAT AGCGATGGTC ATAGAAAAAC GGGGCATATT TATAAACTCT 10650
CAGTTGATCT TAAAATGTGC AAAAGCTGCC GAACTCCTGG GAGTGAGCTC 10700
GAGCCCTGCA GGATCATTGT CACATGTGAG CAAAAGGCCA GCAAAAGGCC 10750
AGGAACCGTA AAAAGGCCGC GTTGCTGGCG TTTTTCCATA GGCTCCGCCC 10800
CCCTGACGAG CATCACAAAA ATCGACGCTC AAGTCAGAGG TGGCGAAACC 10850
CGACAGGACT ATAAAGATAC CAGGCGTTTC CCCCTGGAAG CTCCCTCGTG 10900
CGCTCTCCTG TTCCGACCCT GCCGCTTACC GGATACCTGT CCGCCTTTCT 10950
CCCTTCGGGA AGCGTGGCGC TTTCTCATAG CTCACGCTGT AGGTATCTCA 11000
GTTCGGTGTA GGTCGTTCGC TCCAAGCTGG GCTGTGTGCA CGAACCCCCC 11050
GTTCAGCCCG ACCGCTGCGC CTTATCCGGT AACTATCGTC TTGAGTCCAA 11100
CCCGGTAAGA CACGACTTAT CGCCACTGGC AGCAGCCACT GGTAACAGGA 11150
TTAGCAGAGC GAGGTATGTA GGCGGTGCTA CAGAGTTCTT GAAGTGGTGG 11200
CCTAACTACG GCTACACTAG AAGAACAGTA TTTGGTATCT GCGCTCTGCT 11250
GAAGCCAGTT ACCTTCGGAA AAAGAGTTGG TAGCTCTTGA TCCGGCAAAC 11300
AAACCACCGC TGGTAGCGGT GGTTTTTTTG TTTGCAAGCA GCAGATTACG 11350
CGCAGAAAAA AAGGATCTCA AGAAGATCCT TTGATCTTTT CTACGGGGTC 11400
TGACGCTCAG TGGAACGAAA ACTCACGTTA AGGGATTTTG GTCATGAGAT 11450
TATCAAAAAG GATCTTCACC TAGATCCTTT TAAATTAAAA ATGAAGTTTT 11500
AAATCAAGCC CAATCTGAAT AATGTTACAA CCAATTAACC AATTCTGATT 11550
AGAAAAACTC ATCGAGCATC AAATGAAACT GCAATTTATT CATATCAGGA 11600
TTATCAATAC CATATTTTTG AAAAAGCCGT TTCTGTAATG AAGGAGAAAA 11650
CTCACCGAGG CAGTTCCATA GGATGGCAAG ATCCTGGTAT CGGTCTGCGA 11700
TTCCGACTCG TCCAACATCA ATACAACCTA TTAATTTCCC CTCGTCAAAA 11750
ATAAGGTTAT CAAGTGAGAA ATCACCATGA GTGACGACTG AATCCGGTGA 11800
GAATGGCAAA AGTTTATGCA TTTCTTTCCA GACTTGTTCA ACAGGCCAGC 11850
CATTACGCTC GTCATCAAAA TCACTCGCAT CAACCAAACC GTTATTCATT 11900
CGTGATTGCG CCTGAGCGAG ACGAAATACG CGATCGCTGT TAAAAGGACA 11950
ATTACAAACA GGAATCGAAT GCAACCGGCG CAGGAACACT GCCAGCGCAT 12000
CAACAATATT TTCACCTGAA TCAGGATATT CTTCTAATAC CTGGAATGCT 12050
GTTTTTCCGG GGATCGCAGT GGTGAGTAAC CATGCATCAT CAGGAGTACG 12100
GATAAAATGC TTGATGGTCG GAAGAGGCAT AAATTCCGTC AGCCAGTTTA 12150
GTCTGACCAT CTCATCTGTA ACATCATTGG CAACGCTACC TTTGCCATGT 12200
TTCAGAAACA ACTCTGGCGC ATCGGGCTTC CCATACAAGC GATAGATTGT 12250
CGCACCTGAT TGCCCGACAT TATCGCGAGC CCATTTATAC CCATATAAAT 12300
CAGCATCCAT GTTGGAATTT AATCGCGGCC TCGACGTTTC CCGTTGAATA 12350
TGGCTCATAA CACCCCTTGT ATTACTGTTT ATGTAAGCAG ACAGTTTTAT 12400
TGTTCATGAT GATATATTTT TATCTTGTGC AATGTAACAT CAGAGATTTT 12450
GAGACACGGG CCAGAGCTGC A 12500

### Methods for the Delivery of Exogenous Nucleic Acids to Target Cells

### Transfection Techniques

Techniques that can be used to introduce a transgene, such as a MTM1 transgene described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in e.g., neurons, glial cells, or non-neural cells, such as colon and kidney cells). Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) polyethylenimine, and DEAE-dextran, the use of which as a transfection agent is described in detail, for example, in Gulick et al., Current Protocols in Molecular Biology 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUSTM meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Pharmaceutical Compositions and Routes of Administration

The gene therapy agents described herein may contain a transgene, such as a transgene encoding MTM1 and may be incorporated into a vehicle for administration into a patient, such as a human patient suffering from a neuromuscular disorder (for example, XLMTM). Pharmaceutical compositions containing vectors, such as viral vectors, that contain the transcription regulatory elements (e.g., a desmin promoter) described herein operably linked to a therapeutic transgene can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Viral vectors, such as AAV vectors and others described herein, containing the transcription regulatory element operably linked to a therapeutic transgene may be administered to a patient (e.g., a human patient) by a variety of routes of administration. The route of administration may vary, for example, with the onset and severity of disease, and may include, e.g., intradermal, transdermal, parenteral, intravenous, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Kits

The compositions described herein can be provided in a kit for use in treating a neuromuscular disorder (e.g., XLMTM). The kit may include one or more viral vectors as described herein. The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

### Recommended Clinical Parameters to Consider Prior to Initiation of Daytime Weaning Off of a Mechanical Ventilator

Before considering daytime weaning of a patient off of a mechanically ventilator, a physician of skill in the art should establish patient-specific baselines for airway patency, oxygenation and ventilation capacity, nutritional status, tolerance of rehabilitation therapies, as well as take into consideration broader patient-specific and environmental factors, including the factors listed in Table 2.

TABLE 2
Patient Factors to Consider Before Deciding
to Wean Off Mechanical Ventilation

| | |
|---|---|
| Respiratory function (i.e., efficacy, | Cardiopulmonary interactions (i.e., |
| efficiency, strength and endurance) | rule-out primary or secondary |
| | pulmonary hypertension) |
| Nature of respiratory failure or | Upper airway assessment (i.e., |
| insufficiency | enlarged tonsils/adenoids, |
| | laryngeal and/or tracheomalacia) |
| Respiratory function while awake | Tracheobronchial assessment (i.e., |
| vs asleep | mucosal integrity, laryngeal clefts, |
| | tracheomalacia, granulomas, or |
| | stenosis) |
| Restrictive respiratory mechanics | Nutrition and metabolic demands |
| from long-standing myopathic | |
| changes that warrant ongoing | |
| supports (e.g., scoliosis) | |
| Secretion management (i.e., | Developmental status |
| oropharyngeal, nasopharyngeal, or | |
| tracheal) | |
| Swallow studies | Tolerance of interventions (i.e., |
| | invasive or noninvasive) or need |
| | for other adjuvants |
| Tolerance of intercurrent respiratory | Environmental factors (i.e., caution |
| tract infection without needing | weaning during high-infection |
| ventilator support | seasons or pandemics) |
| Health-related quality of life | Immunization status (i.e., fully |
| | immunized) |
| Chronic lung disease or comorbidities | Planned surgeries in the near future |
| independent of respiratory muscle | |
| function | |

### Recommended Clinical Parameters for Initiation of Daytime Weaning Off of a Mechanical Ventilator

In some embodiments, a patient is considered ready for initiation of daytime weaning off of a mechanical ventilator when the patient's vital signs (e.g., body temperature, heart rate (e.g., pulse), respiratory rate (RR), and blood pressure) and weight are within the age-adjusted norms, or when one or more of the patients, respiratory function indicators (e.g., maximal inspiratory pressure (MIP), maximal expiratory pressure (MEP), positive end-expiratory pressure (PEEP), room air oxygen saturation (SpO₂), transcutaneous CO₂ (TcCO₂), or End Tidal CO₂ (ETCO₂)) or indirect gas exchange markers (e.g., serum bicarbonate levels) are within the measured ranges, as descried herein, during a 12 week assessment following a treatment with a gene therapy product (e.g., an AAV2 encoding MTM1), as described herein.

### I. Vital Signs and Weight

In some embodiments, a patient is considered ready for initiation of daytime weaning off of a mechanical ventilator when the vital signs (e.g., body temperature, heart rate (e.g., pulse), respiratory rate (RR), and blood pressure) and weight of the patient are within age-adjusted norms.

Ia. Body Temperature

In some embodiments, a patient is considered ready for initiation of daytime weaning off of a mechanical ventilator when the patient's body temperature is within the age-adjusted norms, as described herein.

In some embodiments, the temperature of a patient can be measured from the mouth, rectum, axilla (e.g., armpit), ear, or skin. In some embodiments, a patient's oral, rectal, and axillary temperature can be measured with a glass or electronic thermometer.

In some embodiments, the body temperature of a patient is measured orally and is considered to be normal when it falls within the range of about 36.0° C. to 37.5° C. (e.g., about 36.1° C. to about 37.4° C., about 36.2° C. to about 37.3° C., about 36.3° C. to about 37.2° C., about 36.4° C. to about 37.1° C., about 36.5° C. to about 37.0° C., about 36.6° C. to about 36.9° C., or about 36.7° C. to about 36.8° C.).

In some embodiments, the body temperature of a patient is measured rectally and is considered to be normal when it falls within the range of about 36.5° C. to 38.0° C. (e.g., about 36.6° C. to about 37.9° C., about 36.7° C. to about 37.8° C., about 36.8° C. to about 37.7° C., about 36.9° C. to about 37.6° C., about 37.0° C. to about 37.5° C., about 37.1° C. to about 37.4° C., or about 37.2° C. to about 37.3° C.).

In some embodiments, the body temperature of a patient is measured axillary and is considered to be normal when it falls within the range of about 35.5° C. to 37.0° C. (e.g., about 35.6° C. to about 36.9° C., about 35.7° C. to about 36.8° C., about 35.8° C. to about 36.7° C., about 35.9° C. to about 36.6° C., about 36.0° C. to about 36.5° C., about 36.1° C. to about 36.4° C., or about 36.2° C. to about 36.3° C.).

Ib. Heart Rate

In some embodiments, a patient is considered ready for initiation of daytime weaning off of a mechanical ventilator when the patient's heart rate is within the age-adjusted norms, as described herein.

In some embodiments, the heart rate is taken at the radial artery (e.g., wrist). In some embodiments, the heart rate is taken at the brachial artery (e.g., elbow), carotid artery (e.g., neck), popliteal artery (e.g., behind the knee), or at the dorsalis pedis or posterior tibial arteries (e.g., foot).

In some embodiments, the pulse is taken with the index finger and middle finger by pushing with firm yet gentle pressure at the locations described above, and counting the beats felt per 60 seconds. In some embodiments, the heart rate is taken with the index finger and middle finger by pushing with firm yet gentle pressure at the locations described above, and counting the beats felt per 30 seconds and multiplied by two.

In some embodiments, the heart rate is measured by listening directly to the heartbeat using a stethoscope.

In some embodiments the patient is a newborn (e.g., 0-4 months old), an infant (e.g., 0-5 months old), a toddler (e.g., 6-12 months old), a child aged 1-3 years old, a child aged 3-5 years old, a child aged 6-10 years old, an adolescent (e.g., aged 11-14 years old), or an adult (e.g., aged 15+ years old (e.g., aged 16+ years old, aged 17+ years old, aged 18+ years old, aged 19+ years old, aged 20+ years old, aged 21+ years old, aged 22+ years old, aged 23+ years old, aged 24+ years old, aged 25+ years old, aged 26+ years old, aged 27+ years old, aged 28+ years old, aged 29+ years old, aged 30+ years old, aged 40+ years old, aged 50+ years old, aged 60+ years old, aged 70+ years old, aged 80+ years old, or aged 90+ years old)).

In some embodiments, the patient is a newborn (e.g., 0-4 months old), and the patient's heart rate is measured by methods described herein or otherwise and is considered to be within the age-adjusted norm when it falls within the range of about 100 to about 160 beats per minute (bpm) (e.g., about 105 to about 155 bpm, about 110 to about 150 bpm, about 120 to about 150 bpm, about 130 to about 140 bpm, or about 135 bpm).

In some embodiments, the patient is an infant (0-5 months old), and the patient's heart rate is measured by methods described herein or otherwise and is considered to be within the age-adjusted norm when it falls within the range of about 90 to about 150 bpm (e.g., about 95 to about 145 bpm, about 100 to about 140 bpm, about 110 to about 130 bpm, or about 120 bpm).

In some embodiments, the patient is a toddler (6-12 months old), and the patient's heart rate is measured by methods described herein or otherwise and is considered to be within the age-adjusted norm when it falls within the range of about 80 to about 140 bpm (e.g., about 85 to about 135 bpm, about 90 to about 130 bpm, about 100 to about 120 bpm, or about 110 bpm).

In some embodiments, the patient is a child aged 1-3 years old, and the patient's heart rate is measured by methods described herein or otherwise and is considered to be within the age-adjusted norm when it falls within the range of about 80 to about 130 bpm (e.g., about 85 to about 125 bpm, about 90 to about 120 bpm, about 100 to about 110 bpm, or about 115 bpm).

In some embodiments, the patient is a child aged 3-5 years old, and the patient's heart rate is measured by methods described herein or otherwise and is considered to be within the age-adjusted norm when it falls within the range of about 80 to about 120 bpm (e.g., about 85 to about 115 bpm, about 90 to about 110 bpm, or about 100 bpm).

In some embodiments, the patient is a child aged 6-10 years old, and the patient's heart rate is measured by methods described herein or otherwise and is considered to be within the age-adjusted norm when it falls within the range of about 70 to about 110 bpm (e.g., about 75 to about 105 bpm, about 80 to about 100 bpm, or about 90 bpm).

In some embodiments, the patient is an adolescent (e.g., aged 11-14 years old) and, the patient's heart rate is measured by methods described herein or otherwise and is considered to be within the age-adjusted norm when it falls within the range of about 60 to about 105 bpm (e.g., about 65 to about 100 bpm, about 70 to about 95 bpm, about 75 to about 90 bpm, or about 80 to about 85 bpm).

In some embodiments, the patient is an adult (e.g., aged 15+ years old (e.g., aged 16+ years old, aged 17+ years old, aged 18+ years old, aged 19+ years old, aged 20+ years old, aged 21+ years old, aged 22+ years old, aged 23+ years old, aged 24+ years old, aged 25+ years old, aged 26+ years old, aged 27+ years old, aged 28+ years old, aged 29+ years old, aged 30+ years old, aged 40+ years old, aged 50+ years old, aged 60+ years old, aged 70+ years old, aged 80+ years old, or aged 90+ years old)) and, the patient's heart rate is measured by methods described herein or otherwise and is considered to be within the age-adjusted norm when it falls within the range of about 60 to about 100 bpm (e.g., about 65 to about 95 bpm, about 70 to about 90 bpm, or about 80 bpm).

Ic. Respiratory Rate

In some embodiments, a patient is considered ready for initiation of daytime weaning off of a mechanical ventilator when the patient's RR is within the age-adjusted norms, as described herein.

In some embodiments, the RR of a patient can be measured using a stethoscope, or using methods including but not limited to impedance pneumography and capnography.

In some embodiments the patient is a newborn (e.g., 0-6 weeks old), an infant aged 6 weeks-6 months old, a child aged 6 months-3 years old, a child aged 3-6 years old, a child aged 6-10 years old, an adult aged 10-65 years old, an elder aged 65-80, or an elder aged 80+ years old.

In some embodiments, the patient is a newborn (e.g., 0-6 weeks old), and the patient's RR is measured by methods described herein or otherwise and is considered to be within the age-adjusted norm when it falls within the range of about 30 to about 40 breaths (e.g., about 31 to about 39 breaths, about 32 to about 38 breaths, about 33 to about 37 breaths, about 34 to about 36 breaths, or about 35 breaths) per minute.

In some embodiments, the patient is a an infant aged 6 weeks-6 months old, and the patient's RR is measured by methods described herein or otherwise and is considered to be within the age-adjusted norm when it falls within the range of about 25 to about 40 breaths (e.g., about 26 to about 39 breaths, about 27 to about 38 breaths, about 28 to about 37 breaths, about 29 to about 36 breaths, about 30 to about 35 breaths, about 31 to about 34 breaths, or about 32 to about 33 breaths) per minute.

In some embodiments, the patient is a child aged 6 months-3 years old, and the patient's RR is measured by methods described herein or otherwise and is considered to be within the age-adjusted norm when it falls within the range of about 20 to about 30 breaths (e.g., about 21 to about 29 breaths, about 22 to about 28 breaths, about 23 to about 27 breaths, about 29 to about 26 breaths, or about 25 breaths) per minute.

In some embodiments, the patient is a child aged 3-6 years old, and the patient's RR is measured by methods described herein or otherwise and is considered to be within the age-adjusted norm when it falls within the range of about 18 to about 25 breaths (e.g., about 19 to about 24 breaths, about 20 to about 23 breaths, or about 21 to about 22 breaths) per minute.

In some embodiments, the patient is a child aged 6-10 years old, and the patient's RR is measured by methods described herein or otherwise and is considered to be within the age-adjusted norm when it falls within the range of about 17 to about 23 breaths (e.g., about 18 to about 22 breaths, about 19 to about 21 breaths, or about 20 breaths) per minute.

In some embodiments, the patient is an adult aged 10-65 years old, and the patient's RR is measured by methods described herein or otherwise and is considered to be within the age-adjusted norm when it falls within the range of about 15 to about 18 breaths (e.g., about 16 to about 17 breaths) per minute.

In some embodiments, the patient is an adult aged 65+(e.g., 66+, 67+, 68+, 69+, 70+, 75+, 80+, 90+) years old, and patient's the RR is measured by methods described herein or otherwise and is considered to be within the age-adjusted norm when it falls within the range of about 12 to about 28 breaths (e.g., about 13 to about 27 breaths, about 14 to about 26 breaths, about 15 to about 25 breaths, about 16 to about 24 breaths, about 17 to about 23 breaths, about 18 to about 22 breaths, about 19 to about 21 breaths, or about 20) per minute.

Id. Blood Pressure

In some embodiments, a patient is considered ready for initiation of daytime weaning off of a mechanical ventilator when the patient's blood pressure is within the age-adjusted norms, as described herein.

In some embodiments, the blood pressure of a patient can be measured using a sphygmomanometer, with an oscilloscope, or otherwise.

In some embodiments the patient is a newborn (e.g., aged 0-1 month old), an infant (e.g., aged 1-12 months old), a young child (e.g., aged 1-5 years old), an older child (e.g., aged 5+−13 years old), an adolescent (e.g., aged 13+−18 years old), an adult aged 18+−40 years old, an adult aged 40+−60 years old, or an older adult (e.g., aged 60+(e.g., 61+, 62+, 63+, 64+, 65+, 70+, 75+, 80+, 90+) years old).

In some embodiments, the patient is a newborn (e.g., aged 0-1 month old), and the patient's blood pressure is measured by methods described herein or otherwise and is considered to be within the age-adjusted norm when it falls within the range of about 40 to about 80 mmHg (e.g., about 41 to about 79 mmHg, about 42 to about 78 mmHg, about 44 to about 77 mmHg, about 44 to about 76 mmHg, about 45 to about 75 mmHg, about 50 to about 70 mmHg, about 55 to about 65 mmHg, or about 60 mmHg).

In some embodiments, the patient is an infant (e.g., aged 1-12 months old), and the patient's blood pressure is measured by methods described herein or otherwise and is considered to be within the age-adjusted norm when it falls within the range of about 65 to about 100 mmHg (e.g., about 66 to about 99 mmHg, about 67 to about 98 mmHg, about 68 to about 97 mmHg, about 69 to about 96 mmHg, about 70 to about 95 mmHg, about 75 to about 90 mmHg, or about 80 to about 85 mmHg).

In some embodiments, the patient is a young child (e.g., aged 1-5 years old), and the patient's blood pressure is measured by methods described herein or otherwise and is considered to be within the age-adjusted norm when it falls within the range of about 80 to about 115 mmHg (e.g., about 81 to about 114 mmHg, about 82 to about 113 mmHg, about 83 to about 112 mmHg, about 84 to about 111 mmHg, about 85 to about 110 mmHg, about 90 to about 105 mmHg, or about 95 to about 100 mmHg).

In some embodiments, the patient is an older child (e.g., aged 5+−13 years old), and the patient's blood pressure is measured by methods described herein or otherwise and is considered to be within the age-adjusted norm when it falls within the range of about 80 to about 120 mmHg (e.g., about 81 to about 119 mmHg, about 82 to about 118 mmHg, about 83 to about 117 mmHg, about 84 to about 116 mmHg, about 85 to about 115 mmHg, about 90 to about 110 mmHg, about 95 to about 105 mmHg, or about 100 mmHg).

In some embodiments, the patient is an adolescent (e.g., aged 13+−18 years old), and the patient's blood pressure is measured by methods described herein or otherwise and is considered to be within the age-adjusted norm when it falls within the range of about 90 to about 120 mmHg (e.g., about 91 to about 119 mmHg, about 92 to about 118 mmHg, about 93 to about 117 mmHg, about 94 to about 116 mmHg, about 95 to about 115 mmHg, about 100 to about 110 mmHg, or about 105 mmHg).

In some embodiments, the patient is an adult aged 18+−40 years old, and the patient's blood pressure is measured by methods described herein or otherwise and is considered to be within the age-adjusted norm when it falls within the range of about 95 to about 135 mmHg (e.g., about 96 to about 134 mmHg, about 97 to about 133 mmHg, about 98 to about 132 mmHg, about 99 to about 131 mmHg, about 100 to about 130 mmHg, about 105 to about 125 mmHg, about 110 to about 120 mmHg, or about 115 mmHg).

In some embodiments, the patient is an adult aged 40+−60 years old, and the patient's blood pressure is measured by methods described herein or otherwise and is considered to be within the age-adjusted norm when it falls within the range of about 110 to about 145 mmHg (e.g., about 111 to about 144 mmHg, about 112 to about 143 mmHg, about 113 to about 142 mmHg, about 114 to about 141 mmHg, about 115 to about 140 mmHg, about 120 to about 135 mmHg, or about 125 to about 130 mmHg).

In some embodiments, the patient is an older adult (e.g., aged 60+(e.g., 61+, 62+, 63+, 64+, 65+, 70+, 75+, 80+, 90+) years old), and the patient's blood pressure is measured by methods described herein or otherwise and is considered to be within the age-adjusted norm when it falls within the range of about 95 to about 145 mmHg (e.g., about 96 to about 144 mmHg, about 97 to about 143 mmHg, about 98 to about 142 mmHg, about 99 to about 141 mmHg, about 100 to about 140 mmHg, about 105 to about 135 mmHg, about 110 to about 130 mmHg, about 115 to about 125 mmHg, or about 120 mmHg).

Ie. Weight

In some embodiments, a patient is considered ready for initiation of daytime weaning off of a mechanical ventilator when the weight is within the age-adjusted norms, as described herein.

In some embodiments, a male patient is considered ready for initiation of daytime weaning off of a mechanical ventilatory when the weight of the male patient falls within the ranges listed in Table 3.

TABLE 3
Age-Adjusted Norm Weight Range in Males

| | |
|---|---|
| Age | Age-Adjusted Norm Weight Range |
| (months/years) | (pounds) |

| | | |
|---|---|---|
| 0-1 | month | About 5.5 to about 9.5 |
| 1-2 | months | About 7.5 to about 12.5 |
| 2-3 | months | About 9.7 to about 15.4 |
| 3-4 | months | About 11.2 to about 17.4 |
| 4-5 | months | About 12.3 to about 18.9 |
| 5-6 | months | About 13.4 to about 20.3 |
| 6-7 | months | About 14.1 to about 21.4 |
| 7-8 | months | About 14.7 to about 22.5 |
| 8-9 | months | About 15.4 to about 23.1 |
| 9-10 | months | About 15.9 to about 24.0 |
| 10-11 | months | About 16.5 to about 24.7 |
| 11-12 | months | About 16.3 to about 25.4 |
| 1-2 | years | About 17 to about 21 |
| 2-3 | years | About 24 to about 34 |
| 3-4 | years | About 26 to about 38 |
| 4-6 | years | About 30 to about 44 |
| 6-8 | years | About 36 to about 60 |
| 8-10 | years | About 46 to about 78 |
| 10-12 | years | About 54 to about 102 |
| 12-14 | years | About 66 to about 130 |
| 14-16 | years | About 84 to about 160 |
| 16-18 | years | About 104 to about 186 |
| 18+ | years | About 116 to about 202 |

In some embodiments, a female patient is considered ready for initiation of daytime weaning off of a mechanical ventilatory when the weight of the female patient falls within the ranges listed in Table 4.

TABLE 4
Age-Adjusted Norm Weight Range in Females

| | |
|---|---|
| Age | Age-Adjusted Norm Weight Range |
| (months/years) | (lbs) |

| | | |
|---|---|---|
| 0-1 | month | About 5.3 to about 9.3 |
| 1-2 | months | About 7.0 to about 11.9 |
| 2-3 | months | About 8.8 to about 14.3 |
| 3-4 | months | About 10.1 to about 16.3 |
| 4-5 | months | About 11.2 to about 17.9 |
| 5-6 | months | About 12.1 to about 19.1 |
| 6-7 | months | About 12.8 to about 20.3 |
| 7-8 | months | About 13.4 to about 21.1 |
| 8-9 | months | About 13.8 to about 22.0 |
| 9-10 | months | About 14.5 to about 22.9 |
| 10-11 | months | About 15.0 to about 23.6 |
| 11-12 | months | About 15.4 to about 24.3 |
| 1-2 | years | About 15 to about 20 |
| 2-3 | years | About 22 to about 32 |
| 3-4 | years | About 26 to about 38 |
| 4-6 | years | About 28 to about 44 |
| 6-8 | years | About 36 to about 60 |
| 8-10 | years | About 44 to about 80 |
| 10-12 | years | About 54 to about 106 |
| 12-14 | years | About 68 to about 136 |
| 14-16 | years | About 84 to about 160 |
| 16-18 | years | About 94 to about 172 |
| 18+ | years | About 100 to about 178 |

II. Respiratory function

In some embodiments, a patient is considered ready for initiation of daytime weaning off of a mechanical ventilator when one or more respiratory function indicators (e.g., MIP, MEP, PEEP, SpO₂, TcCO₂, or ETCO₂) are within the measured ranges, as descried herein.

IIa. Inspiratory Pressure

In some embodiments, a patient is considered ready for initiation of daytime weaning off of a mechanical ventilator when the patient's requirement for MIP on the ventilator is greater than −50 cmH₂O (e.g., greater than −49 cmH₂O, greater than −48 cmH₂O, greater than −47 cmH₂O, greater than −46 cmH₂O, greater than −45 cmH₂O, greater than −40 cmH₂O, greater than −35 cmH₂O, greater than −30 cmH₂O, greater than −20 cmH₂O, greater than −10 cmH₂O, or greater than 0.0 cmH₂O).

IIb. Expiratory Pressure

In some embodiments, a patient is considered ready for initiation of daytime weaning off of a mechanical ventilator when the patient's requirement for MEP on the ventilator is greater than 40 cmH₂O (e.g., greater than 41 cmH₂O, greater than 42 cmH₂O, greater than 43 cmH₂O, greater than 44 cmH₂O, greater than 45 cmH₂O, greater than 50 cmH₂O, greater than 55 cmH₂O, greater than 60 cmH₂O, greater than 70 cmH₂O, or greater than 80 cmH₂O).

IIc. Positive End-Expiratory Pressure

In some embodiments, a patient is considered ready for initiation of daytime weaning off of a mechanical ventilator when the patient's requirement for PEEP on the ventilator is less than or equal to 5 cmH₂O (e.g., less than or equal to 5 cmH₂O, less than or equal to 4 cmH₂O, less than or equal to 3 cmH₂O, less than or equal to 2 cmH₂O, less than or equal to 1 cmH₂O, or less than or equal to 0 cmH₂O).

IId. Room Air Oxygen Saturation

In some embodiments, a patient is considered ready for initiation of daytime weaning off of a mechanical ventilator when the patient's SpO₂ is greater than 94% (e.g., greater than 95%, greater than 96%, greater than 97%, greater than 98%, or greater than 99%).

IIe. Transcutaneous CO₂

In some embodiments, a patient is considered ready for initiation of daytime weaning off of a mechanical ventilator when the patient's TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg).

IIf. End Tidal CO₂

In some embodiments, a patient is considered ready for initiation of daytime weaning off of a mechanical ventilator when the patient's ETCO₂ is about 35 to about 45 mmHg (e.g., about 36 to about 44 mmHg, about 37 to about 43 mmHg, about 38 to about 42 mmHg, about 39 to about 41 mmHg, or about 40 mmHg).

### III. Indirect Gas Exchange

In some embodiments, a patient is considered ready for initiation of daytime weaning off of a mechanical ventilator when one or more indirect gas exchange markers (e.g., serum bicarbonate) are within the measured ranges, as descried herein.

IIIa. Serum Bicarbonate

In some embodiments, a patient is considered ready for initiation of daytime weaning off of a mechanical ventilator when the patient's serum bicarbonate levels are about 22 to about 27 mEq/L (e.g., about 23 to about 26 mEq/L, or about 24 to about 25 mEq/L).

### VI. Additional Considerations: Clinical Judgement

In some embodiments, a patient is considered ready for initiation of daytime weaning off of a mechanical ventilator when one or more clinical parameters are taken into consideration, including week-to-week clinical improvements in motor milestones (e.g., head control, sitting, voluntary grasp, ability to kick in supine, rolling, crawling or bottom shuffling, standing, and walking), vocalization, coughing, secretions, or a motor function score on the CHOP INTEND.

### Recommended Clinical Parameters for Continuation of Daytime Weaning Off of a Mechanical Ventilator

In some embodiments, a patient is considered ready for continuation of daytime weaning off of a mechanical ventilator when one or more of the patient's vital signs (e.g., RR), respiratory function indicators (e.g., SpO₂, TcCO₂), and clinical parameters (e.g., intercostal retraction, tachypnea, respiratory paradox, or phase delay) are within the measured ranges, as descried herein, during the assessment of a video recording of a respiratory sprinting trial; when one or more of the patient's vital signs (e.g., RR) or respiratory function indicators (e.g., TcCO₂, ETCO₂, or SpO₂) are within the measured ranges, as descried herein during nocturnal respiration monitoring; or when one or more of the patient's vital signs (e.g., RR), respiratory function indicators (e.g., TcCO₂, petCO₂, ptcCO₂, P02, or SpO₂), or the apnea-hyponea index (AHI) are within the measured ranges, as descried herein, when a polysomnogram (PSG) is performed with the tracheostomy open for invasively ventilated patients or with the mask off for noninvasively ventilated patients.

### I. Respiratory Sprinting Trial(s)

In some embodiments, a patient is considered ready for continuation of daytime weaning off of a mechanical ventilator when one or more of the respiratory function indicators (e.g., SpO₂, TcCO₂) are within the measured ranges described herein during the assessment of a video recording of a respiratory sprinting trial or when none of the clinical parameters (e.g., intercostal retraction, tachypnea, respiratory paradox, or phase delay) are observed during the assessment of a video recording of a respiratory sprinting trial.

In some embodiments, the duration of a respiratory sprinting trial is about 15 to about 30 minutes (e.g., about 16 to about 29 minutes, about 17 to about 28 minutes, about 18 to about 27 minutes, about 19 to about 26 minutes, about 20 to about 25 minutes, or about 20 minutes) long.

In some embodiments, the duration of a respiratory sprinting trial is progressively increased, for example from 24 minutes to 25 minutes, every 3 to 4 days.

Ia. Respiratory function

In some embodiments, a patient is considered ready for continuation of daytime weaning off of a mechanical ventilator when one or more respiratory function indicators (e.g., SpO₂ or TcCO₂) are within the measured ranges, as descried herein.

Iai. SpO₂

In some embodiments, a patient is considered ready for continuation of daytime weaning off of a mechanical ventilator when the patient's SpO₂ is observed to be greater than 94% (e.g., greater than 95%, greater than 96%, greater than 97%, greater than 98%, or greater than 99%) during a video recording of a respiratory sprinting trial.

In some embodiments, a patient is considered ready for continuation of daytime weaning off of a mechanical ventilator when the patient's SpO₂ does not differ by greater than 3% (e.g., does not differ by greater than 4%, does not differ by greater than 5%, does not differ by greater than 6%, does not differ by greater than 7%, does not differ by greater than 8%, does not differ by greater than 9%, does not differ by greater than 10%, does not differ by greater than 15%, does not differ by greater than 20%, or does not differ by greater than 30%) from the awake baseline during a video recording of a respiratory sprinting trial.

Iaii. TcCO₂

In some embodiments, a patient is considered ready for continuation of daytime weaning off of a mechanical ventilator when the patient's TcCO₂ is less than 45 mmHg (e.g., less than 44 mmHg, less than 43 mmHg, less than 42 mmHg, less than 41 mmHg, or less than 40 mmHg) during a video recording of a respiratory sprinting trial.

In some embodiments, a patient is considered ready for continuation of daytime weaning off of a mechanical ventilator when the patient's TcCO₂ did not increase by 10 mmHg or greater (e.g., did not increase by 11 mmHg or greater, did not increase by 12 mmHg or greater, did not increase by 13 mmHg or greater, did not increase by 14 mmHg or greater, did not increase by 15 mmHg or greater, did not increase by 20 mmHg or greater, did not increase by 25 mmHg or greater, or did not increase by 30 mmHg or greater) from the awake baseline during a video recording of a respiratory sprinting trial.

Ib. Clinical Judgement

In some embodiments, a patient is considered ready for continuation of daytime weaning off of a mechanical ventilator when no distress is observed during a video recording of a respiratory sprinting trial.

In some embodiments, a patient is considered ready for continuation of daytime weaning off of a mechanical ventilator when no intercostal retraction is observed during a video recording of a respiratory sprinting trial.

In some embodiments, a patient is considered ready for continuation of daytime weaning off of a mechanical ventilator when no tachypnea is observed during a video recording of a respiratory sprinting trial.

In some embodiments, a patient is considered ready for continuation of daytime weaning off of a mechanical ventilator when no respiratory paradox is observed during a video recording of a respiratory sprinting trial.

In some embodiments, a patient is considered ready for continuation of daytime weaning off of a mechanical ventilator when no phase delay is observed during a video recording of a respiratory sprinting trial.

### II. Nocturnal Respiration Monitoring

In some embodiments, a patient is considered ready for continuation of daytime weaning off of a mechanical ventilator when one or more of the patient's vital signs (e.g., RR) or respiratory function indicators (e.g., TcCO₂, ETCO₂, SpO₂) are within the measured ranges, as descried herein, during the assessment of nocturnal monitoring.

IIa. Vital Signs

In some embodiments, a patient is considered ready for continuation of daytime weaning off of a mechanical ventilator when one or more of the patient's vital signs (e.g., RR) are within the measured ranges, as descried herein, during the assessment of nocturnal monitoring.

IIai. Respiration Rate

In some embodiments, the RR of a patient can be measured using a stethoscope, or using methods including but not limited to impedance pneumography and capnography.

In some embodiments the patient is a newborn (e.g., 0-6 weeks old), an infant aged 6 weeks-6 months old, a child aged 6 months-3 years old, a child aged 3-6 years old, a child aged 6-10 years old, an adult aged 10-65 years old, an elder aged 65-80, or an elder aged 80+ years old.

In some embodiments, the patient is a newborn (e.g., 0-6 weeks old), and the patient's RR is measured by methods described herein or otherwise and is considered to be within the age-adjusted norm when it falls within the range of about 30 to about 40 breaths (e.g., about 31 to about 39 breaths, about 32 to about 38 breaths, about 33 to about 37 breaths, about 34 to about 36 breaths, or about 35 breaths) per minute.

In some embodiments, the patient is a an infant aged 6 weeks-6 months old, and the patient's RR is measured by methods described herein or otherwise and is considered to be within the age-adjusted norm when it falls within the range of about 25 to about 40 breaths (e.g., about 26 to about 39 breaths, about 27 to about 38 breaths, about 28 to about 37 breaths, about 29 to about 36 breaths, about 30 to about 35 breaths, about 31 to about 34 breaths, or about 32 to about 33 breaths) per minute.

In some embodiments, the patient is a child aged 6 months-3 years old, and the patient's RR is measured by methods described herein or otherwise and is considered to be within the age-adjusted norm when it falls within the range of about 20 to about 30 breaths (e.g., about 21 to about 29 breaths, about 22 to about 28 breaths, about 23 to about 27 breaths, about 29 to about 26 breaths, or about 25 breaths) per minute.

In some embodiments, the patient is a child aged 3-6 years old, and the patient's RR is measured by methods described herein or otherwise and is considered to be within the age-adjusted norm when it falls within the range of about 18 to about 25 breaths (e.g., about 19 to about 24 breaths, about 20 to about 23 breaths, or about 21 to about 22 breaths) per minute.

In some embodiments, the patient is a child aged 6-10 years old, and the patient's RR is measured by methods described herein or otherwise and is considered to be within the age-adjusted norm when it falls within the range of about 17 to about 23 breaths (e.g., about 18 to about 22 breaths, about 19 to about 21 breaths, or about 20 breaths) per minute.

In some embodiments, the patient is an adult aged 10-65 years old, and the patient's RR is measured by methods described herein or otherwise and is considered to be within the age-adjusted norm when it falls within the range of about 15 to about 18 breaths (e.g., about 16 to about 17 breaths) per minute.

In some embodiments, the patient is an adult aged 65+(e.g., 66+, 67+, 68+, 69+, 70+, 75+, 80+, 90+) years old, and patient's the RR is measured by methods described herein or otherwise and is considered to be within the age-adjusted norm when it falls within the range of about 12 to about 28 breaths (e.g., about 13 to about 27 breaths, about 14 to about 26 breaths, about 15 to about 25 breaths, about 16 to about 24 breaths, about 17 to about 23 breaths, about 18 to about 22 breaths, about 19 to about 21 breaths, or about 20) per minute.

IIb. Respiratory Function

In some embodiments, a patient is considered ready for continuation of daytime weaning off of a mechanical ventilator when one or more respiratory function indicators (e.g., SpO₂, TcCO₂, or ETCO₂) are within the measured ranges, as descried herein, during nocturnal respiration monitoring.

IIbi. Transcutaneous CO₂

In some embodiments, a patient is considered ready for continuation of daytime weaning off of a mechanical ventilator when the patient's TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

IIbii. End Tidal CO₂

In some embodiments, a patient is considered ready for continuation of daytime weaning off of a mechanical ventilator when the patient's ETCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

IIbiii. Oxygen Saturation

In some embodiments, a patient is considered ready for continuation of daytime weaning off of a mechanical ventilator when the patient's SpO₂ is greater than 94% (e.g., greater than 95%, greater than 96%, greater than 97%, greater than 98%, or greater than 99%) during nocturnal respiration monitoring.

### III. Polysomnogram

In some embodiments, the respiration of a polysomnogram (PSG) is performed while a patient is off of the mechanical ventilator. In some embodiments, the patient is an invasively ventilated patient, wherein the PSG is performed while the patient is off of the mechanical ventilator and with the tracheostomy open for. In some embodiments, the patient is a noninvasively ventilated patient, wherein the PSG is performed and measured nocturnally while the patient is off of the mechanical ventilator.

In some embodiments, a patient is considered ready for continuation of daytime weaning off of a mechanical ventilator when one or more of the patient's vital signs (e.g., respiration rate), respiratory function indicators (e.g., TcCO₂, petCO₂, ptcCO₂, P02, or SpO₂), or the AHI are within the measured ranges, as descried herein, during a PSG.

In some embodiments, a PSG is replaced by measurement of TcCO₂ (i.e., using a digital monitoring system) during nocturnal respiration monitoring. In some embodiments, a patient is considered ready for continuation of daytime weaning off of a mechanical ventilator when the patient's TcCO₂ has been monitored nocturnally about 2-3 nights (e.g., about 2 or about 3 nights) and the TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

IIIa. Vital Signs

In some embodiments, a patient is considered ready for continuation of daytime weaning off of a mechanical ventilator when one or more of the patient's vital signs (e.g., RR) are within the measured ranges, as descried herein, during a PSG.

IIIai. Respiration rate

In some embodiments, the RR of a patient can be measured using a stethoscope, or using methods including but not limited to impedance pneumography and capnography.

In some embodiments the patient is a newborn (e.g., 0-6 weeks old), an infant aged 6 weeks-6 months old, a child aged 6 months-3 years old, a child aged 3-6 years old, a child aged 6-10 years old, an adult aged 10-65 years old, an elder aged 65-80, or an elder aged 80+ years old.

In some embodiments, the patient is a newborn (e.g., 0-6 weeks old), and the patient's RR is measured by methods described herein or otherwise and is considered to be within the age-adjusted norm when it falls within the range of about 30 to about 40 breaths (e.g., about 31 to about 39 breaths, about 32 to about 38 breaths, about 33 to about 37 breaths, about 34 to about 36 breaths, or about 35 breaths) per minute.

In some embodiments, the patient is a an infant aged 6 weeks-6 months old, and the patient's RR is measured by methods described herein or otherwise and is considered to be within the age-adjusted norm when it falls within the range of about 25 to about 40 breaths (e.g., about 26 to about 39 breaths, about 27 to about 38 breaths, about 28 to about 37 breaths, about 29 to about 36 breaths, about 30 to about 35 breaths, about 31 to about 34 breaths, or about 32 to about 33 breaths) per minute.

In some embodiments, the patient is a child aged 6 months-3 years old, and the patient's RR is measured by methods described herein or otherwise and is considered to be within the age-adjusted norm when it falls within the range of about 20 to about 30 breaths (e.g., about 21 to about 29 breaths, about 22 to about 28 breaths, about 23 to about 27 breaths, about 29 to about 26 breaths, or about 25 breaths) per minute.

In some embodiments, the patient is a child aged 3-6 years old, and the patient's RR is measured by methods described herein or otherwise and is considered to be within the age-adjusted norm when it falls within the range of about 18 to about 25 breaths (e.g., about 19 to about 24 breaths, about 20 to about 23 breaths, or about 21 to about 22 breaths) per minute.

In some embodiments, the patient is a child aged 6-10 years old, and the patient's RR is measured by methods described herein or otherwise and is considered to be within the age-adjusted norm when it falls within the range of about 17 to about 23 breaths (e.g., about 18 to about 22 breaths, about 19 to about 21 breaths, or about 20 breaths) per minute.

In some embodiments, the patient is an adult aged 10-65 years old, and the patient's RR is measured by methods described herein or otherwise and is considered to be within the age-adjusted norm when it falls within the range of about 15 to about 18 breaths (e.g., about 16 to about 17 breaths) per minute.

In some embodiments, the patient is an adult aged 65+(e.g., 66+, 67+, 68+, 69+, 70+, 75+, 80+, 90+) years old, and patient's the RR is measured by methods described herein or otherwise and is considered to be within the age-adjusted norm when it falls within the range of about 12 to about 28 breaths (e.g., about 13 to about 27 breaths, about 14 to about 26 breaths, about 15 to about 25 breaths, about 16 to about 24 breaths, about 17 to about 23 breaths, about 18 to about 22 breaths, about 19 to about 21 breaths, or about 20) per minute.

IIIb. Apnea-Hyponea Index

In some embodiments, a patient is considered ready for continuation of daytime weaning off of a mechanical ventilator when the AHI is less than 5 events/hour (e.g., less than 4 events/hour, less than 3 events/hour, less than 2 events/hour, or less than 1 events/hour) when a PSG is performed with the tracheostomy open for invasively ventilated patients or with the mask off for noninvasively ventilated patients.

IIIc. Respiratory Function

In some embodiments, a patient is considered ready for continuation of daytime weaning off of a mechanical ventilator when one or more respiratory function indicators (e.g., TcCO₂, petCO₂ or ptcCO₂, or ETCO₂) are within the measured ranges, as descried herein, when a PSG is performed with the tracheostomy open for invasively ventilated patients or with the mask off for noninvasively ventilated patients.

IIIci. Transcutaneous CO₂

In some embodiments, a patient is considered ready for continuation of daytime weaning off of a mechanical ventilator when the patient's TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) when a PSG is performed with the tracheostomy open for invasively ventilated patients or with the mask off for noninvasively ventilated patients.

In some embodiments, a patient is considered ready for continuation of daytime weaning off of a mechanical ventilator when the patient's TcCO₂ did not increase by 10 mmHg or greater (e.g., did not increase by 11 mmHg or greater, did not increase by 12 mmHg or greater, did not increase by 13 mmHg or greater, did not increase by 14 mmHg or greater, did not increase by 15 mmHg or greater, did not increase by 20 mmHg or greater, did not increase by 25 mmHg or greater, or did not increase by 30 mmHg or greater) from awake baseline when a PSG is performed with the tracheostomy open for invasively ventilated patients or with the mask off for noninvasively ventilated patients.

IIIcii. End-Tidal CO₂ or Partial Pressure of CO₂

In some embodiments, a patient is considered ready for continuation of daytime weaning off of a mechanical ventilator either when a patient's petCO₂ or ptcCO₂ are within the measured ranges, as descried herein, during a PSG.

In some embodiments, a patient is considered ready for continuation of daytime weaning off of a mechanical ventilator when the patient's petCO₂ or ptcCO₂ is less than 50 mmHg (e.g., less than 49 mmHg, less than 48 mmHg, less than 47 mmHg, less than 46 mmHg, less than 45 mmHg, less than 40 mmHg, less than 35 mmHg, less than 30 mmHg, less than 20 mmHg, or less than 10 mmHg) when a PSG is performed with the tracheostomy open for invasively ventilated patients or with the mask off for noninvasively ventilated patients.

In some embodiments, a patient is considered ready for continuation of daytime weaning off of a mechanical ventilator when the patient's petCO₂ or ptcCO₂ does not increase by greater than 10 mmHg (e.g., did not increase by greater than 11 mmHg, did not increase during sleep by greater than 11 mmHg, did not increase by greater than 11 mmHg, did not increase by greater than 11 mmHg, did not increase by greater than 11 mmHg, did not increase by greater than 11 mmHg, did not increase by greater than 11 mmHg, or did not increase by greater than 11 mmHg) from the awake baseline when a PSG is performed with the tracheostomy open for invasively ventilated patients or with the mask off for noninvasively ventilated patients.

IIIciii. Oxygen Saturation

In some embodiments, a patient is considered ready for continuation of daytime weaning off of a mechanical ventilator when the patient's SpO₂ is greater than 94% (e.g., greater than 95%, greater than 96%, greater than 97%, greater than 98%, or greater than 99%) when a PSG is performed with the tracheostomy open for invasively ventilated patients or with the mask off for noninvasively ventilated patients.

### Naptime Weaning Off of a Mechanical Ventilator

In some embodiments, when a patient has successfully discontinued ventilator support during waking daytime hours, a physician of skill in the art may consider initiating a process of weaning the patient off of a mechanical ventilator during naptimes.

In some embodiments, a pulse oximeter is used during naptimes to monitor a patient for oxygen desaturation and increases in heart rate.

In some embodiments, a physician of skill in the art may take a patient's home-based monitorization of TcCO₂ into account in determining whether or not a patient is ready for initiating a process of weaning off of a mechanical ventilator during naptimes.

In some embodiments, a patient is considered not ready for continuation of naptime weaning off of a mechanical ventilator when tachypnea is observed in the patient during naptime weaning.

In some embodiments, a patient is considered not ready for continuation of naptime weaning off of a mechanical ventilator when the patient's tachypnea is observed during naptime weaning.

In some embodiments, a patient is considered not ready for continuation of naptime weaning off of a mechanical ventilator when a patient's SpO₂ is less than 95% (e.g., less than 94%, less than 93%, less than 92%, less than 91%, less than 90%, less than 85%, less than 80%, less than 70%, or less than 60%) during naptime weaning.

In some embodiments, a patient is considered not ready for continuation of naptime weaning off of a mechanical ventilator when a patient's heart rate increases by more than 20 bpm (e.g., more than 21 bpm, more than 22 bpm, more than 23 bpm, more than 24 bpm, more than 25 bpm, more than 30 bpm, or more than 40 bmp) from the awake baseline during naptime weaning.

In some embodiments, a patient is considered not ready for continuation of naptime weaning off of a mechanical ventilator when a patient's TcCO₂ is greater than 50 mmHg (e.g., greater than 51 mmHg, greater than 52 mmHg, greater than 53 mmHg, greater than 54 mmHg, greater than 55 mmHg, greater than 60 mmHg, greater than 65 mmHg, greater than 70 mmHg, or greater than 80 mmHg) during naptime weaning.

In some embodiments, a patient is considered not ready for continuation of naptime weaning off of a mechanical ventilator when a patient's TcCO₂ increases by 10 mmHg or greater (e.g., increases by 11 mmHg or greater, increases by 12 mmHg or greater, increases by 13 mmHg or greater, increases by 14 mmHg or greater, increases by 15 mmHg or greater, increases by 20 mmHg or greater, increases by 25 mmHg or greater, or increases by 30 mmHg or greater) from the awake baseline during naptime weaning.

### Nocturnal Weaning Off of a Mechanical Ventilator

In some embodiments, when a patient has successfully discontinued ventilator support during both waking daytime and naptime hours, a physician of skill in the art may consider initiating a process of weaning the patient off of a mechanical ventilator during nighttime.

In some embodiments, a patient is considered ready for continuation of nighttime weaning off of a mechanical ventilator when one or more of the patient's vital signs (e.g., RR) or respiratory function indicators (e.g., TcCO₂, ETCO₂, or SpO₂) are within the measured ranges, as descried herein during nocturnal respiration monitoring; or when one or more of the patient's vital signs (e.g., RR), respiratory function indicators (e.g., TcCO₂, end-tidal CO₂ (petCO₂), partial pressure of CO₂ (ptcCO₂), PO₂, or SpO₂), or the AHI are within the measured ranges, as descried herein, when a PSG is performed with the tracheostomy open for invasively ventilated patients or with the mask off for noninvasively ventilated patients.

Recommended Clinical Parameters for Continuation of Daytime Weaning Off of a Mechanical Ventilator In some embodiments, a patient is considered ready for continuation of nighttime weaning off of a mechanical ventilator when one or more of the patient's vital signs (e.g., RR) or respiratory function indicators (e.g., TcCO₂, ETCO₂, or SpO₂) are within the measured ranges, as descried herein during nocturnal respiration monitoring; or when one or more of the patient's vital signs (e.g., RR), respiratory function indicators (e.g., TcCO₂, petCO₂, ptcCO₂, PO₂, or SpO₂), or the AHI is within the measured ranges, as descried herein, when a PSG is performed with the tracheostomy open for invasively ventilated patients or with the mask off for noninvasively ventilated patients.

### I. Nocturnal Respiration Monitoring

In some embodiments, a patient is considered ready for continuation of nighttime weaning off of a mechanical ventilator when one or more of the patient's vital signs (e.g., RR) or respiratory function indicators (e.g., TcCO₂, ETCO₂, SpO₂) are within the measured ranges, as descried herein, during the assessment of nocturnal monitoring.

Ia. Vital Signs

In some embodiments, a patient is considered ready for continuation of nighttime weaning off of a mechanical ventilator when one or more of the patient's vital signs (e.g., RR) are within the measured ranges, as descried herein, during the assessment of nocturnal monitoring.

Iai. Respiration Rate

In some embodiments, the RR of a patient can be measured using a stethoscope, or using methods including but not limited to impedance pneumography and capnography.

In some embodiments the patient is a newborn (e.g., 0-6 weeks old), an infant aged 6 weeks-6 months old, a child aged 6 months-3 years old, a child aged 3-6 years old, a child aged 6-10 years old, an adult aged 10-65 years old, an elder aged 65-80, or an elder aged 80+ years old.

In some embodiments, the patient is a newborn (e.g., 0-6 weeks old), and the patient's RR is measured by methods described herein or otherwise and is considered to be within the age-adjusted norm when it falls within the range of about 30 to about 40 breaths (e.g., about 31 to about 39 breaths, about 32 to about 38 breaths, about 33 to about 37 breaths, about 34 to about 36 breaths, or about 35 breaths) per minute.

In some embodiments, the patient is a an infant aged 6 weeks-6 months old, and the patient's RR is measured by methods described herein or otherwise and is considered to be within the age-adjusted norm when it falls within the range of about 25 to about 40 breaths (e.g., about 26 to about 39 breaths, about 27 to about 38 breaths, about 28 to about 37 breaths, about 29 to about 36 breaths, about 30 to about 35 breaths, about 31 to about 34 breaths, or about 32 to about 33 breaths) per minute.

In some embodiments, the patient is a child aged 6 months-3 years old, and the patient's RR is measured by methods described herein or otherwise and is considered to be within the age-adjusted norm when it falls within the range of about 20 to about 30 breaths (e.g., about 21 to about 29 breaths, about 22 to about 28 breaths, about 23 to about 27 breaths, about 29 to about 26 breaths, or about 25 breaths) per minute.

In some embodiments, the patient is a child aged 3-6 years old, and the patient's RR is measured by methods described herein or otherwise and is considered to be within the age-adjusted norm when it falls within the range of about 18 to about 25 breaths (e.g., about 19 to about 24 breaths, about 20 to about 23 breaths, or about 21 to about 22 breaths) per minute.

In some embodiments, the patient is a child aged 6-10 years old, and the patient's RR is measured by methods described herein or otherwise and is considered to be within the age-adjusted norm when it falls within the range of about 17 to about 23 breaths (e.g., about 18 to about 22 breaths, about 19 to about 21 breaths, or about 20 breaths) per minute.

In some embodiments, the patient is an adult aged 10-65 years old, and the patient's RR is measured by methods described herein or otherwise and is considered to be within the age-adjusted norm when it falls within the range of about 15 to about 18 breaths (e.g., about 16 to about 17 breaths) per minute.

In some embodiments, the patient is an adult aged 65+(e.g., 66+, 67+, 68+, 69+, 70+, 75+, 80+, 90+) years old, and patient's the RR is measured by methods described herein or otherwise and is considered to be within the age-adjusted norm when it falls within the range of about 12 to about 28 breaths (e.g., about 13 to about 27 breaths, about 14 to about 26 breaths, about 15 to about 25 breaths, about 16 to about 24 breaths, about 17 to about 23 breaths, about 18 to about 22 breaths, about 19 to about 21 breaths, or about 20) per minute.

Ib. Respiratory Function

In some embodiments, a patient is considered ready for continuation of nighttime weaning off of a mechanical ventilator when one or more respiratory function indicators (e.g., SpO₂, TcCO₂, or ETCO₂) are within the measured ranges, as descried herein, during nocturnal respiration monitoring.

Ibi. Transcutaneous CO₂

In some embodiments, a patient is considered ready for continuation of nighttime weaning off of a mechanical ventilator when the patient's TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

Ibii. End Tidal CO₂

In some embodiments, a patient is considered ready for continuation of nighttime weaning off of a mechanical ventilator when the patient's ETCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

Ibiii. Oxygen Saturation

In some embodiments, a patient is considered ready for continuation of nighttime weaning off of a mechanical ventilator when the patient's SpO₂ is greater than 94% (e.g., greater than 95%, greater than 96%, greater than 97%, greater than 98%, or greater than 99%) during nocturnal respiration monitoring.

### II. Polysomnogram

In some embodiments, the respiration of a PSG is performed while a patient is off of the mechanical ventilator. In some embodiments, the patient is an invasively ventilated patient, wherein the PSG is performed while the patient is off of the mechanical ventilator and with the tracheostomy open for. In some embodiments, the patient is a noninvasively ventilated patient, wherein the PSG is performed and measured nocturnally while the patient is off of the mechanical ventilator.

In some embodiments, a patient is considered ready for continuation of nighttime weaning off of a mechanical ventilator when one or more of the patient's vital signs (e.g., RR), respiratory function indicators (e.g., TcCO₂, petCO₂, ptcCO₂, P02, or SpO₂), or AHI are within the measured ranges, as descried herein, during a PSG.

In some embodiments, a PSG is replaced by measurement of TcCO₂ (i.e., using a digital monitoring system) during nocturnal respiration monitoring. In some embodiments, a patient is considered ready for continuation of nighttime weaning off of a mechanical ventilator when the patient's TcCO₂ has been monitored nocturnally about 2-3 nights (e.g., about 2 or about 3 nights) and the TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) during nocturnal respiration monitoring.

IIa. Vital Signs

In some embodiments, a patient is considered ready for continuation of nighttime weaning off of a mechanical ventilator when one or more of the patient's vital signs (e.g., RR) are within the measured ranges, as descried herein, during a PSG.

IIai. Respiration Rate

In some embodiments, the RR of a patient can be measured using a stethoscope, or using methods including but not limited to impedance pneumography and capnography.

In some embodiments the patient is a newborn (e.g., 0-6 weeks old), an infant aged 6 weeks-6 months old, a child aged 6 months-3 years old, a child aged 3-6 years old, a child aged 6-10 years old, an adult aged 10-65 years old, an elder aged 65-80, or an elder aged 80+ years old.

In some embodiments, the patient is a newborn (e.g., 0-6 weeks old), and the patient's RR is measured by methods described herein or otherwise and is considered to be within the age-adjusted norm when it falls within the range of about 30 to about 40 breaths (e.g., about 31 to about 39 breaths, about 32 to about 38 breaths, about 33 to about 37 breaths, about 34 to about 36 breaths, or about 35 breaths) per minute.

In some embodiments, the patient is a an infant aged 6 weeks-6 months old, and the patient's RR is measured by methods described herein or otherwise and is considered to be within the age-adjusted norm when it falls within the range of about 25 to about 40 breaths (e.g., about 26 to about 39 breaths, about 27 to about 38 breaths, about 28 to about 37 breaths, about 29 to about 36 breaths, about 30 to about 35 breaths, about 31 to about 34 breaths, or about 32 to about 33 breaths) per minute.

In some embodiments, the patient is a child aged 6 months-3 years old, and the patient's RR is measured by methods described herein or otherwise and is considered to be within the age-adjusted norm when it falls within the range of about 20 to about 30 breaths (e.g., about 21 to about 29 breaths, about 22 to about 28 breaths, about 23 to about 27 breaths, about 29 to about 26 breaths, or about 25 breaths) per minute.

In some embodiments, the patient is a child aged 3-6 years old, and the patient's RR is measured by methods described herein or otherwise and is considered to be within the age-adjusted norm when it falls within the range of about 18 to about 25 breaths (e.g., about 19 to about 24 breaths, about 20 to about 23 breaths, or about 21 to about 22 breaths) per minute.

In some embodiments, the patient is a child aged 6-10 years old, and the patient's RR is measured by methods described herein or otherwise and is considered to be within the age-adjusted norm when it falls within the range of about 17 to about 23 breaths (e.g., about 18 to about 22 breaths, about 19 to about 21 breaths, or about 20 breaths) per minute.

In some embodiments, the patient is an adult aged 10-65 years old, and the patient's RR is measured by methods described herein or otherwise and is considered to be within the age-adjusted norm when it falls within the range of about 15 to about 18 breaths (e.g., about 16 to about 17 breaths) per minute.

In some embodiments, the patient is an adult aged 65+(e.g., 66+, 67+, 68+, 69+, 70+, 75+, 80+, 90+) years old, and patient's the RR is measured by methods described herein or otherwise and is considered to be within the age-adjusted norm when it falls within the range of about 12 to about 28 breaths (e.g., about 13 to about 27 breaths, about 14 to about 26 breaths, about 15 to about 25 breaths, about 16 to about 24 breaths, about 17 to about 23 breaths, about 18 to about 22 breaths, about 19 to about 21 breaths, or about 20) per minute.

IIb. Apnea-Hyponea Index

In some embodiments, a patient is considered ready for continuation of nighttime weaning off of a mechanical ventilator when the AHI is less than 5 events/hour (e.g., less than 4 events/hour, less than 3 events/hour, less than 2 events/hour, or less than 1 events/hour) when a PSG is performed with the tracheostomy open for invasively ventilated patients or with the mask off for noninvasively ventilated patients.

IIc. Respiratory Function

In some embodiments, a patient is considered ready for continuation of nighttime weaning off of a mechanical ventilator when one or more respiratory function indicators (e.g., TcCO₂, petCO₂ or ptcCO₂, or ETCO₂) are within the measured ranges, as descried herein, when a PSG is performed with the tracheostomy open for invasively ventilated patients or with the mask off for noninvasively ventilated patients.

IIci. Transcutaneous CO₂

In some embodiments, a patient is considered ready for continuation of nighttime weaning off of a mechanical ventilator when the patient's TcCO₂ is about 35 to about 45 mmHg (e.g., about 36 to about 44 mmHg, about 37 to about 43 mmHg, about 38 to about 42 mmHg, about 39 to about 41 mmHg, or about 40 mmHg) when a PSG is performed with the tracheostomy open for invasively ventilated patients or with the mask off for noninvasively ventilated patients.

In some embodiments, a patient is considered ready for continuation of nighttime weaning off of a mechanical ventilator when the patient's TcCO₂ did not increase by 10 mmHg or greater (e.g., did not increase by 11 mmHg or greater, did not increase by 12 mmHg or greater, did not increase by 13 mmHg or greater, did not increase by 14 mmHg or greater, did not increase by 15 mmHg or greater, did not increase by 20 mmHg or greater, did not increase by 25 mmHg or greater, or did not increase by 30 mmHg or greater) from awake baseline when a PSG is performed with the tracheostomy open for invasively ventilated patients or with the mask off for noninvasively ventilated patients.

IIcii. End-Tidal CO₂ or Partial Pressure of CO₂

In some embodiments, a patient is considered ready for continuation of nighttime weaning off of a mechanical ventilator either when a patient's petCO₂ or ptcCO₂ are within the measured ranges, as descried herein, during a PSG.

In some embodiments, a patient is considered ready for continuation of nighttime weaning off of a mechanical ventilator when the patient's petCO₂ or ptcCO₂ is less than 50 mmHg (e.g., less than 49 mmHg, less than 48 mmHg, less than 47 mmHg, less than 46 mmHg, less than 45 mmHg, less than 40 mmHg, less than 35 mmHg, less than 30 mmHg, less than 20 mmHg, or less than 10 mmHg) when a PSG is performed with the tracheostomy open for invasively ventilated patients or with the mask off for noninvasively ventilated patients.

In some embodiments, a patient is considered ready for continuation of nighttime weaning off of a mechanical ventilator when the patient's petCO₂ or ptcCO₂ does not increase by greater than 10 mmHg (e.g., did not increase by greater than 11 mmHg, did not increase during sleep by greater than 11 mmHg, did not increase by greater than 11 mmHg, did not increase by greater than 11 mmHg, did not increase by greater than 11 mmHg, did not increase by greater than 11 mmHg, did not increase by greater than 11 mmHg, or did not increase by greater than 11 mmHg) from the awake baseline when a PSG is performed with the tracheostomy open for invasively ventilated patients or with the mask off for noninvasively ventilated patients.

IIciii. Oxygen Saturation

In some embodiments, a patient is considered ready for continuation of nighttime weaning off of a mechanical ventilator when the patient's SpO₂ is greater than 94% (e.g., greater than 95%, greater than 96%, greater than 97%, greater than 98%, or greater than 99%) when a PSG is performed with the tracheostomy open for invasively ventilated patients or with the mask off for noninvasively ventilated patients.

### EXAMPLES

The following examples are put forth so as to provide those of ordinary skill in the art with a description of how the compositions and methods described herein may be used and evaluated, and are intended to be purely exemplary of the invention and are not intended to limit the scope of what the inventors regard as their invention.

### Example 1. An Algorithm for Discontinuing Mechanical Ventilation in Pediatric Patients with X-Linked Myotubular Myopathy

### Abstract

X-linked myotubular myopathy (XLMTM) is a rare, life-threatening congenital myopathy characterized in most patients by profound muscle weakness and hypotonia at birth resulting in severe respiratory insufficiency, inability to sit up, stand or walk, and early mortality. At birth, 85-90% of XLMTM patients require mechanical ventilation, with more than half requiring invasive ventilator support. As a result, a priori expectations for improvement in neuromuscular-derived respiratory failure and aerodigestive risks for these ventilator-dependent children were low. Nevertheless, in the recent ASPIRO trial, administration of a novel gene therapy in children with XLMTM surpassed expectations, leading to unprecedented and rapid improvements in respiratory and neuromuscular function, including ventilator independence, sitting without support, standing, and walking. Hence, the robust outcomes assessments in the investigational protocol were paired with a rigorous weaning algorithm to match clinical efficacy observed, minimize potential morbidity, and assist clinicians and families with emerging capacity and needs in the patient (e.g., increased muscle strength, ability to vocalize). However, there was no precedent for weaning chronically ventilated patients with congenital neuromuscular diseases off mechanical ventilation. In the absence of published guidance, an algorithm was developed to help clinicians treating XLMTM patients in ASPIRO safely wean children off mechanical ventilation in response to their improving respiratory muscle strength. The algorithm provides recommendations for assessing weaning readiness, a stepwise approach to weaning, and monitoring of patients during and after the weaning process.

### INTRODUCTION

X-linked myotubular myopathy (XLMTM) is a rare, life-threatening congenital myopathy arising from mutations in the MTM1 gene, resulting in absent or dysfunctional myotubularin protein. XLMTM is characterized in most patients by profound muscle weakness and hypotonia at birth resulting in severe respiratory insufficiency; absent or transient achievement of motor milestones including sitting, standing or walking; and early death. At birth, most XLMTM patients (85-90%) require mechanical ventilation, approximately two-thirds of whom require ventilation for >16 hours/day, with some requiring 24-hour ventilation, and more than half requiring invasive respiratory support. Most patients with XLMTM require invasive respiratory support permanently.

In the recently reported ASPIRO trial, a single infusion of resamirigene bilparvovec gene therapy resulted in unprecedented improvements in respiratory and neuromuscular function in children with XLMTM, all of whom required at least 12 hours per day of ventilator support before treatment. Additional improvements following treatment included ventilator independence, spontaneous secretion management, sitting without support, standing and walking. Chronically ventilated patients with congenital neuromuscular diseases (NMD), similar to XLMTM, had never before been weaned off mechanical ventilation. These patients typically require permanent ventilator support close to 24 hours per day due to neuromuscular insufficiency, restrictive lung disease (e.g., due to atrophied diaphragms and lung scarring from aspiration pneumonitis or recurrent pneumonias), impaired secretion clearance, scoliosis. Nonetheless, evidence of readiness for weaning off mechanical ventilation emerged almost immediately following gene transfer in ASPIRO.

With no existing clinical guidance for ventilator weaning and/or discontinuation in this population, guidance was developed for clinicians treating XLMTM patients in the ASPIRO trial to safely wean children off mechanical ventilation and potential tracheal decannulation for those with invasive supports, in accordance with their response to gene therapy.

An international group of pulmonologists and respiratory physiologists (RJG, ND, LE, EKF, CL, VM, GFR, CS, BKS, FS, SP, SR, GFP) was convened in Boston in April 2018 to devise an algorithm to safely wean (reduce) the number of hours of ventilation. The algorithm was informed by XLMTM patient profiles and aggregated data on ventilator dependence, respiratory pressures, secretion management, capnography, and polysomnography studies. The first draft algorithm, based on expert consensus at that meeting, was refined with input from additional experts in the fields of pediatric pulmonology, sleep medicine, respiratory physiology and neuromuscular disorders (RA, HS, WM), and supplemented by a review of recently available literature.

Recommendations are provided for 1) assessing weaning readiness, 2) a stepwise approach to weaning, and, 3) a multidisciplinary monitoring framework for patients during and after the weaning process. It is understood that clinical care for children with NMD varies between providers and institutions worldwide, with variable interpretation of, and adherence to, established respiratory care guidelines. Without precedence for weaning, greater variability in practices and extrapolation, perhaps inappropriately yet unavoidably, from unrelated conditions would be anticipated. Thus, the guidelines outlined here acknowledge differences in existing care regimens while highlighting developmentally appropriate pathways and creating clinical boundaries to direct and, potentially limit, weaning.

### Weaning Patients Off Chronic Mechanical Ventilation

Clinical experience with weaning pediatric patients with NMD off mechanical ventilation is limited and largely confined to extubation in the intensive care unit (i.e., not chronically ventilated patients). The vast majority of experience in weaning pediatric patients from chronic invasive or non-invasive respiratory support comes from children with congenital malformations (i.e., tracheomalacia, congenital heart disease); self-limited, acquired neuromuscular conditions (e.g., Guillain-Barré syndrome or spinal cord injury); and those with chronic lung disease of prematurity. In patients with prolonged respiratory failure, the term “weaning” is apropos, as it describes a gradual process of improving the load-to-capacity ratio of the mechanical and gas exchange capacity of the respiratory system to enable spontaneous and sustainable respiration.

Overall, fewer studies have examined ventilator-induced neuromuscular (particularly diaphragm) weakness in children than in adults. Ventilator-induced neuromuscular (particularly diaphragm) weakness is common among mechanically ventilated adults in the critical care setting and contributes to prolonged weaning, extubation failure, and higher mortality. In children, studies indicate that diaphragm atrophy is associated with prolonged recovery and use of noninvasive ventilation in the acute care setting. It is thought that maturational changes in the respiratory mechanics and diaphragm histology throughout infancy may be a factor. As children grow, mechanics change and anabolic demands differ dramatically from adults. In the pediatric acute care setting, risk factors for reintubation include acute neurologic disease, lower pre-extubation MIP, impaired spontaneous secretion clearance, postextubation upper airway obstruction, higher pre-extubation positive end-expiratory pressure (PEEP) settings, higher postextubation pressure rate product, and high postextubation phase angle.

Due to the static or progressive nature of most NMD, there is limited published experience with weaning NMD patients off mechanical ventilation. Weaning off transtracheal supports is often not considered or has often required transition to noninvasive supportive ventilation. This requires parents (or adult patients) to accept different risks and monitoring, demonstration of developmental capacity and willingness to tolerate noninvasive ventilation, and adaptation of care.

Weaning chronic invasive ventilator support is a slow process. In children with chronic lung disease of prematurity, the median age of liberation from respiratory support is 24 months. Patients with NMD do not typically demonstrate rapid improvement (or any improvement at all) in respiratory muscle function, especially those dependent on invasive ventilation.

### Determining Readiness of Patients to Wean Off Mechanical Ventilation

Before considering weaning any mechanically ventilated patient, baselines for airway patency, oxygenation and ventilation capacity, nutritional status, tolerance of rehabilitation therapies may be established and broader patient and environmental factors may be considered (Table 2). A multidisciplinary approach is advisable. For XLMTM patients in the ASPIRO trial, some treatment-emergent adverse events required intensification of immunosuppression, thereby increasing risk of respiratory tract infection in an already at-risk population. It is anticipated that similar considerations would apply to future gene therapy in other NMD.

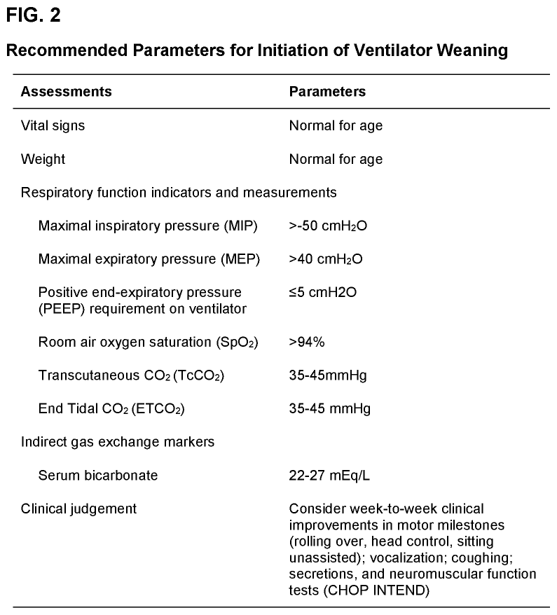

Parameters and values that indicate readiness for reduction in mechanical ventilation support are shown in FIG. 2. These include respiratory function tests, gas exchange markers, airway patency indicators, nocturnal respiration parameters, polysomnogram results, and clinical judgement. FIG. 2 also provides guidance on monitoring of patients during the weaning process and after the patient has successfully discontinued mechanical ventilation.

### Recommendations for Weaning Patients Off Mechanical Ventilation

Weaning is the process of decreasing the amount of support that the patient receives from the mechanical ventilator, so that the patient assumes a greater proportion of the ventilatory effort. The objective is to assess the probability that mechanical ventilation can be successfully discontinued. Multiple respiratory evaluations may be conducted prior to a weaning assessment being attempted. Based on the rapid clinical response of the first patients treated with resamirigene bilparvovec gene therapy, a weaning assessment may be attempted after week 12. Discontinuing mechanical ventilation is a multi-step process, consisting of readiness testing, weaning, and reassessment. Unlike approaches to other forms of chronic lung disease or acute care weaning, this guideline supports sequential weans of transtracheal supports and transition to a spontaneous respiratory mode without an anticipated transition to noninvasive ventilation as an intermediate step. The reasoning is multifactorial. Mask interfaces required for noninvasive ventilation (NIV) may not be tolerated by infants and toddlers unaccustomed to masks. NIV carries potential risk for compromised skin integrity, aspiration, and other aerodigestive considerations, and could perpetuate a tracheocutaneous fistula/tract in patients with tracheostomy, requiring surgical intervention. Most importantly, the need for NIV implies ongoing respiratory insufficiency, which supports more conditioning, time, and assessment of capacity to tolerate other stressors (e.g., respiratory infections).

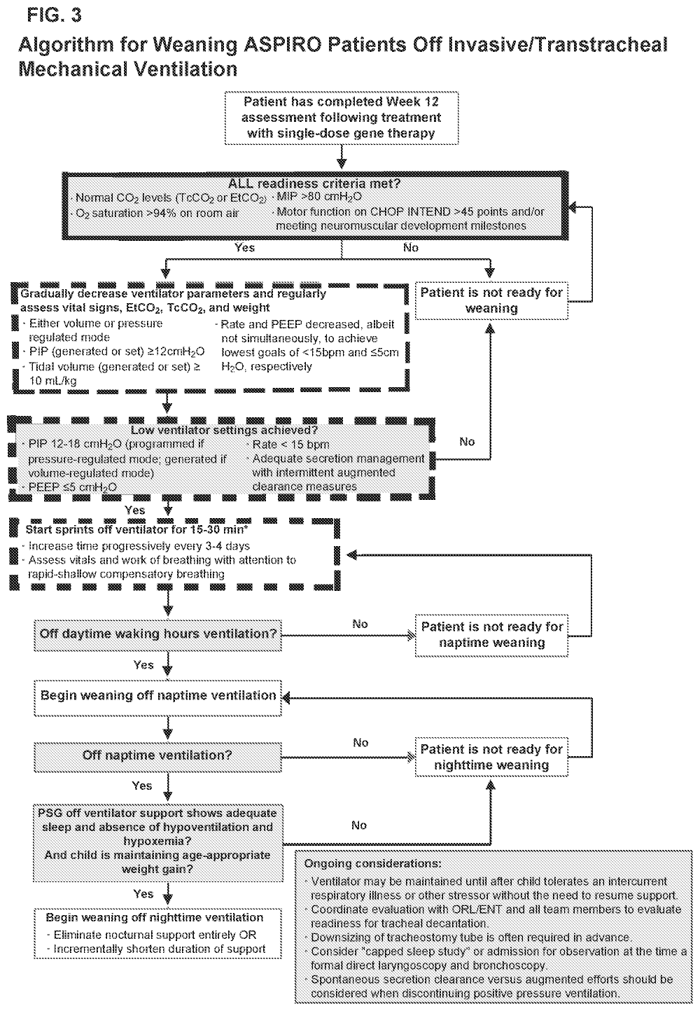

The algorithm in FIG. 3 presents a stepwise approach to weaning that involves assessment of respiratory function and readiness at each step during the weaning process.

Patients who meet the readiness criteria outlined in FIG. 2 can move forward with daytime weaning. These recommended values are proposed in the context of the ASPIRO clinical trial. They are intended to be more conservative, recognizing the uncertainty of the clinical trial and intuitive need to optimize respiratory support with dynamic metabolic and catabolic demands in response to the gene therapy. Weaning usually involves a gradual reduction in ventilator support (i.e., pressure/volume/rate) for patients on higher settings and continuous support, followed by a progressive sprinting off the ventilator.

Throughout the weaning process, providers and parents may be attentive to the work of breathing, compensatory tachypnea, compensatory tachycardia, and other clinical evidence of distress. Assessment of spontaneous secretion clearance or, conversely, need for additional cough augmentation or suctioning may also serve to guide the process. A pulse oximeter may be used to monitor oxygen saturation and heart rate. Stopping the daytime weaning process may be considered if oxygen saturation decreases to <95% or by 3-4% from baseline; or if heart rate increases more than 20 bpm from baseline. (Baseline is defined as the time of weaning assessment with reassessment at subsequent trial encounters.) Heart rate increases can be an indicator of cardiac compensation for respiratory insufficiency or carbon dioxide retention or for indolent hypercapnia prior to oxygen desaturation. It is important to note that patients with neuromuscular weakness may not show the typical signs of respiratory failure, such as retractions and compensatory tachypnea; thus other signs including tachycardia and looking anxious may prompt the clinician to stop the weaning process.

### Ventilator Settings During Weaning

Reduction of ventilator settings will depend on the mode of mechanical ventilatory support. The basic concept is to decrease settings progressively and monitor adequate gas exchange and vital signs. Weaning can be achieved by decreasing either the peak inspiratory pressure (PIP), tidal volume (TV) or rate. In general, one parameter may be weaned at the same time to avoid weaning failure due to excessive respiratory muscle overload. This will also facilitate interpretation of the response of different parameters to the weaning process.

The adjustment of ventilator settings during weaning may be tailored to each patient, and the child's overall wellness in response to ventilator changes may be carefully assessed. First, the degree of ventilator support over the previous 24 hours may be considered. Time on the ventilator or ventilator pressure over time may be reduced, depending on how much support the patient has recently needed. In general, while working on daytime weaning, the nighttime ventilator settings may be maintained to provide effective recruitment and gas exchange for recovery to maximize respiratory muscle work performance during the day.

The patient's baseline tolerance for spontaneous breathing can be determined by testing sprint duration in the clinic and progressively adding time off the ventilator from there (e.g., 1 hour, then 2 hours, then 3 hours and so on) in 30-60 minute increments. Providers can assist families in determining which daytime weaning schema is preferable—single sessions of longer duration or multiple “sprints” of shorter duration—until these sessions merge. From a neuromuscular perspective, the latter has implicit benefit with muscle conditioning and interval rests.

Minimum ventilator settings in the range of 10-15 cmH₂O for PIP and 4-5 cmH₂O for PEEP are prudent, prior to daytime “sprints.” Generated tidal volumes may be trended to avoid atelectasis. A spontaneous mode is preferable, in the absence of other central neurologic issues. Some providers may prefer a low mandatory rate 5-10 bpm on hybrid support (e.g., average volume-assured pressure support) overnight with complete liberation by day, which would be appropriate as well.

### Naptime and Nighttime Weaning

When the patient has successfully discontinued ventilator support during waking daytime hours, initiating the process of weaning off ventilator support during naptimes may be considered to assess respiratory efficiency during sleep. A pulse oximeter may also be used during naptimes to monitor for oxygen desaturation and heart rate increases; the latter may be a surrogate for cardiorespiratory compensation or indolent hypercapnia prior to desaturation. If available, a home-based TcCO₂ monitor can be beneficial, though it is possible that variability in experience using these monitors may present challenges. Stopping the naptime weaning process may be considered if there is tachypnea, oxygen saturation drops to <95%, heart rate increases more than 20 bpm from baseline, or TcCO₂ is above 50 mmHg or increases 10 mmHg above the awake baseline.

When the patient has successfully discontinued ventilator support during both waking and napping daytime hours, the process of discontinuing nighttime ventilator support may begin. Before eliminating nighttime ventilator support, a polysomnogram, which monitors for adequate gas exchange and assesses for adequate sleep (i.e., awakenings), is the gold standard for assessing sleep disordered breathing.

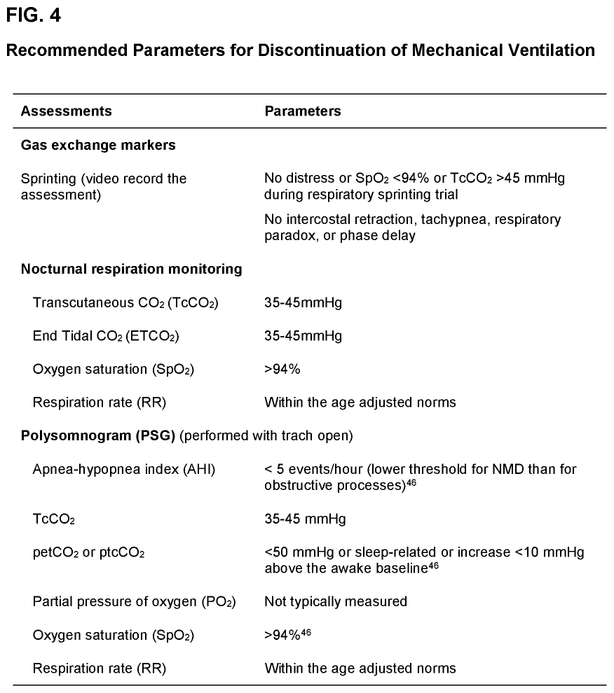

Nighttime weaning can be accomplished either by reducing the number of ventilator support hours per night at regular intervals or by eliminating nocturnal support entirely. Most clinicians prefer to eliminate nocturnal support in a single step because weaning at hourly increments puts a large burden of lost sleep on patients and their families/caregivers. The need for “nocturnal conditioning” also suggests that the child may not be ready for discontinuation and might require resumption of supports with any stressor. The nocturnal respiration monitoring and polysomnogram parameters recommended for discontinuation of mechanical ventilation are shown in FIG. 4. The polysomnogram may be performed off the ventilator with the tracheostomy open for invasively ventilated patients or with the mask off for noninvasively ventilated patients. If a polysomnogram is not feasible, the best surrogate is nighttime TcCO₂ monitoring (i.e., using a digital monitoring system), in patients with access to this technology, for 2-3 nights off the ventilator before discontinuing the ventilator entirely. Regardless, nocturnal monitoring of oximetry, heart rate, estimates of respiration rate and, if available, home EtCO₂ monitoring may be continued after nighttime ventilator support is discontinued for the first 6-8 weeks after the study. Alternatively, the patient could be admitted to the hospital for night time close monitoring and blood gases in the morning. A follow-up polysomnogram is recommended for patients who have changes in clinical course, including mild desaturations, poor weight gain or mood changes. Thereafter, correlation of gross motor trajectory with standardized neuromuscular measures (e.g., CHOP INTEND or Bayley Scales of Infant and Toddler Development 3rd edition, gross motor function domain) or tracking attainment of major motor milestones (e.g., sitting unassisted for >30 seconds, standing, walking with and without support), and general clinical status can inform the need for follow-up polysomnogram.

### Assessing Weaning Outcomes

With any weaning strategy, the clinician may determine whether weaning was a success or a failure. Objective criteria that may indicate weaning failure include tachypnea, respiratory distress (use of accessory muscles, thoracoabdominal paradox, and diaphoresis), hemodynamic changes (tachycardia, hypertension), oxyhemoglobin desaturation, hypercapnia, failure to thrive (weight loss or slowing of growth) and changes in mental status (somnolence, agitation, or more subtle behavioral changes). In addition, parents and providers may continue to be attentive to and report daytime symptomology, such as fatigue and headache or intolerance of activities and therapy.

### Additional Care Considerations

During the weaning process, a multidisciplinary approach may be employed such that clinical teams, research teams, primary investigators and the pulmonary/respiratory rehabilitation team be closely aligned and regularly sharing data and care information. It will also be important to communicate with the patient's other health care providers, including physical therapist, speech pathologist, and nutritionist, to determine therapy modifications based on the patient's improvements.

Secretion Management and/or Intercurrent Illness or Infection

Augmented secretion clearance may facilitate ventilator weaning and sprinting periods. Chest physiotherapy, mechanical cough assistance, tracheal suctioning, and/or manual bag breaths may be advisable before a sprinting trial to minimize airway obstruction and atelectasis at the outset. However, the increased need for intervention during a sprint may suggest the need to resume ventilator support, as it is an indicator of insufficient capacity.

Weaning may be paused during illness (infectious or unrelated reactive airway disease flares) and capacity reassessed following recovery. If the child is off the ventilator but still has a tracheostomy, providers may consider resumption of support; that is, if supplemental oxygen is needed or the child is in distress, resuming ventilator support may be first line. An increase in frequency or severity of illness during the weaning period may be indicative of an increased need for respiratory support. There is also an appreciation that patients who have sustained lung injury from chronic aspiration or recurrent pneumonia may have limited potential to wean off supports. It may be necessary to initiate a parallel approach to chronic parenchymal lung disease along with the NMD guidelines. In this instance, ventilation may not be necessary long-term, rather supplementary oxygen may be the only requirement. However, clinicians may heed the warning of hypoxia as it can reflect ventilation-perfusion mismatching, and oxygen supplementation can mask hypoventilation.

### Weight Maintenance

Failure to thrive during weaning in the absence of other factors may imply that the caloric demands of weaning and spontaneous breathing are exceeding caloric intake. The answer may not be empiric increase in calories, as this may increase CO₂ burden and consequently the need to breathe more. Close monitoring from an experienced team is warranted.

### Daytime Activity

Close attention to tiredness and ensuring adequate rest may be important. If activity declines during weaning, then the ventilator may be utilized to aid in recovery or to revert to the prior level of support. Signs of fatigue, which may manifest in the inability to tolerate routine physical therapy sessions, may indicate that weaning is proceeding too quickly. Ideally, the child may be able to maintain his/her prior activity levels during weaning.

### Interventions in the Presence of Fixed-Restrictive Lung Disease

The need for spinal instrumentation to address neuromuscular scoliosis (growth rods, vertical expandable prosthetic titanium rib, etc.) may necessitate prolonged post-operative respiratory support that would be facilitated by a tracheostomy and a ventilator or noninvasive ventilation. For patients whose thoracic cage is fixed and restrictive, there may be a degree of persistent respiratory insufficiency that is not recoverable independent of muscle strength. This may prompt discussions about longer-term options and would be informed by polysomnogram and other clinical indicators, as delineated above.

### CONCLUSIONS

Using this weaning protocol, seven of the first ten children with XLMTM treated with gene therapy in the ongoing ASPIRO trial have safely discontinued mechanical ventilator support. Close follow-up and regular respiratory assessments will be needed given the unknown trajectory and durability of respiratory outcomes after weaning these patients off mechanical ventilation. It is acknowledged that this algorithm was evaluated only in an XLMTM population who received resamirigene bilparvovec gene therapy. However, it is believed that its applicability can be extrapolated to other congenital NMD, given that the respiratory failure physiopathology and recovery are similar across NMD. The guidelines proposed in this manuscript can be a valuable tool for pediatric NMD patients undergoing treatment with investigational therapies.

### Example 2. Treatment of a Disorder in Human Patients by Administration of a Pseudotyped AAV2/8 Vector Including a Nucleic Acid Sequence Encoding a Myotubularin 1 Gene Operably Linked to a Desmin Promotor and the Mechanical Ventilator Weaning Regime in Accordance with the Disclosure

Using the compositions and methods of the disclosure, a patient having a disorder (e.g., X-linked myotubular myopathy (XLMTM)) may be administered a pseudotyped AAV2/8 vector including a nucleic acid sequence encoding an Myotubularin 1 (MTMI) gene operably linked to a desmin promotor (FIG. 1).

To assess the patient's readiness for daytime weaning off of mechanical ventilation following administration of a therapeutic agent described above, a physician of skill in the art may analyze one or more of the following parameters: (1) determining that the patient exhibits vital signs and a weight that are within the age-adjusted norms; (2) determining that the patient exhibits a motor function score on the Children's Hospital of Philadelphia Infant Test of Neuromuscular Disorders (CHOP INTEND) that is >45 or that neuromuscular development milestones have been met; (3) determining that the patient exhibits a maximal inspiratory pressure that is >−50 cmH₂O on the ventilator; (4) determining that the patient exhibits a maximal expiratory pressure that is >40 cmH₂O on the ventilator; (5) determining that the patient exhibits a positive end-expiratory pressure that is cmH₂O on the ventilator; (6) determining that the patient exhibits a saturation of room air oxygen (SpO₂) that is >94%; (7) determining that the patient exhibits a transcutaneous CO₂ (TcCO₂) that is within 35-45 mmHg; (8) determining that the patient exhibits an End Tidal CO₂ (ETCO₂) that is within 35-45 mmHg; or (9) determining that the patient exhibits serum bicarbonate levels that are within 22-27 mEq/L (FIG. 2). Readiness for daytime weaning off of mechanical ventilation may be assessed, for example, by determining: that the patient exhibits vital signs and a weight that are within the age-adjusted norms, that the patient exhibits a motor function score on the CHOP INTEND that is >45 or that neuromuscular development milestones have been met, and that the patient exhibits a maximal inspiratory pressure that is >−50 cmH₂O on the ventilator.

If, upon assessment of the aforementioned parameters, a physician of skill in the art determines that a patient is ready for daytime weaning off of mechanical ventilation, following daytime weaning off of mechanical ventilation to assess the patient's readiness for continuation of daytime weaning off of mechanical ventilation, a physician of skill in the art may analyze one or more of the following parameters: (1) determining that the patient exhibits a respiration rate (RR) that is within the age-adjusted norms when respiration is monitored nocturnally; (2) determining that the patient exhibits no distress in the video recording of a respiratory sprinting trial; (3) determining that the patient exhibits a RR that is within the age-adjusted norm when a polysomnogram is performed with the tracheostomy open; (4) determining that the patient exhibits a TcCO₂ that is within 35-45 mmHg when respiration is monitored nocturnally; (5) an ETCO₂ that is within 35-45 mmHg when respiration is monitored nocturnally; (6) an SpO₂ that is >94 when respiration is monitored nocturnally; (7) determining that the patient exhibits an apnea-hypopnea index (AHI) that is <5 events/hour when a polysomnogram is performed with the tracheostomy open; (8) determining that the patient exhibits a TcCO₂ that is within 35-45 mmHg or that did not increases by 10 mmHg or greater above the awake baseline when a polysomnogram is performed with the tracheostomy open; (9) determining that the patient exhibits a petCO₂ or a ptcCO₂ that is <50 mmHg or that did not increase by greater than 10 mmHg from the awake baseline during sleep when a polysomnogram is performed with the tracheostomy open; (10) determining that the patient exhibits no intercostal retraction in the video recording of a respiratory sprinting trial; (11) determining that the patient exhibits no tachypnea in the video recording of a respiratory sprinting trial; (12) determining that the patient exhibits no respiratory paradox in the video recording of a respiratory sprinting trial; (13) determining that the patient exhibits no phase delay in the video recording of a respiratory sprinting trial; (14) determining that the patient exhibits an SpO₂ that is <94% or that does not differ by greater than 3% from baseline in the video recording of a respiratory sprinting trial; or (15) determining that the patient exhibits a TcCO₂ that is >45 mmHg or that did not increase by 10 mmHg or greater above the awake baseline in the video recording of a respiratory sprinting trial (FIG. 4). Readiness for the continuation of daytime weaning off of mechanical ventilation may be assessed, for example, by determining: that the patient exhibits a RR that is within the age-adjusted norms when respiration is monitored nocturnally, that the patient exhibits no distress in the video recording of a respiratory sprinting trial, and that the patient exhibits a TcCO₂ that is within 35-45 mmHg when respiration is monitored nocturnally.

### Example 3. Treatment of X-Linked Myotubular Myopathy in Human Patients by Administration of a Pseudotyped AAV2/8 Vector Including a Nucleic Acid Sequence Encoding an MTMI Gene Operably Linked to a Desmin Promotor and the Mechanical Ventilator Weaning Regime in Accordance with the Disclosure

Using the compositions and methods of the disclosure, a patient having a neuromuscular disorder (e.g., XLMTM) may be administered a pseudotyped AAV2/8 vector including a nucleic acid sequence encoding an MTMI gene operably linked to a desmin promotor.

To assess the patient's readiness for daytime weaning off of mechanical ventilation following administration of a therapeutic agent described above, a physician of skill in the art may analyze one or more of the following parameters: (1) determining that the patient exhibits vital signs and a weight that are within the age-adjusted norms; (2) determining that the patient exhibits a motor function score on the CHOP INTEND that is >45 or that neuromuscular development milestones have been met; (3) determining that the patient exhibits a maximal inspiratory pressure that is >−50 cmH₂O on the ventilator; (4) determining that the patient exhibits a maximal expiratory pressure that is >40 cmH₂O on the ventilator; (5) determining that the patient exhibits a positive end-expiratory pressure that is cmH₂O on the ventilator; (6) determining that the patient exhibits a SpO₂ that is >94%; (7) determining that the patient exhibits a TcCO₂ that is within 35-45 mmHg; (8) determining that the patient exhibits an ETCO₂ that is within 35-45 mmHg; or (9) determining that the patient exhibits serum bicarbonate levels that are within 22-27 mEq/L. Readiness for daytime weaning off of mechanical ventilation may be assessed, for example, by determining: that the patient exhibits vital signs and a weight that are within the age-adjusted norms, that the patient exhibits a motor function score on the CHOP INTEND that is >45 or that neuromuscular development milestones have been met, and that the patient exhibits a maximal inspiratory pressure that is >−50 cmH₂O on the ventilator.

If, upon assessment of the aforementioned parameters, a physician of skill in the art determines that a patient is ready for daytime weaning off of mechanical ventilation, following daytime weaning off of mechanical ventilation to assess the patient's readiness for continuation of daytime weaning off of mechanical ventilation, a physician of skill in the art may analyze one or more of the following parameters: (1) determining that the patient exhibits a RR that is within the age-adjusted norms when respiration is monitored nocturnally; (2) determining that the patient exhibits no distress in the video recording of a respiratory sprinting trial; (3) determining that the patient exhibits a RR that is within the age-adjusted norm when a polysomnogram is performed with the tracheostomy open; (4) determining that the patient exhibits a TcCO₂ that is within 35-45 mmHg when respiration is monitored nocturnally; (5) an ETCO₂ that is within 35-45 mmHg when respiration is monitored nocturnally; (6) a SpO₂ that is >94 when respiration is monitored nocturnally; (7) determining that the patient exhibits an AHI that is <5 events/hour when a polysomnogram is performed with the tracheostomy open; (8) determining that the patient exhibits a TcCO₂ that is within 35-45 mmHg or that did not increases by 10 mmHg or greater above the awake baseline when a polysomnogram is performed with the tracheostomy open; (9) determining that the patient exhibits a petCO₂ or a ptcCO₂ that is <50 mmHg or that did not increase by greater than 10 mmHg from the awake baseline during sleep when a polysomnogram is performed with the tracheostomy open; (10) determining that the patient exhibits no intercostal retraction in the video recording of a respiratory sprinting trial; (11) determining that the patient exhibits no tachypnea in the video recording of a respiratory sprinting trial; (12) determining that the patient exhibits no respiratory paradox in the video recording of a respiratory sprinting trial; (13) determining that the patient exhibits no phase delay in the video recording of a respiratory sprinting trial; (14) determining that the patient exhibits an SpO₂ that is <94% or that does not differ by greater than 3% from baseline in the video recording of a respiratory sprinting trial; or (15) determining that the patient exhibits a TcCO₂ that is >45 mmHg or that did not increase by 10 mmHg or greater above the awake baseline in the video recording of a respiratory sprinting trial. Readiness for the continuation of daytime weaning off of mechanical ventilation may be assessed, for example, by determining: that the patient exhibits a RR that is within the age-adjusted norms when respiration is monitored nocturnally, that the patient exhibits no distress in the video recording of a respiratory sprinting trial, and that the patient exhibits a TcCO₂ that is within 35-45 mmHg when respiration is monitored nocturnally.

### Example 4. Treatment of X-Linked Myotubular Myopathy in Human Patients by Administration of Resamirigene Bilparvovec and the Mechanical Ventilator Weaning Regime in Accordance with the Disclosure

Using the compositions and methods of the disclosure, a patient having a neuromuscular disorder (e.g., XLMTM) may be administered resamirigene bilparvovec.

To assess the patient's readiness for daytime weaning off of mechanical ventilation following administration of a therapeutic agent described above, a physician of skill in the art may analyze one or more of the following parameters: (1) determining that the patient exhibits vital signs and a weight that are within the age-adjusted norms; (2) determining that the patient exhibits a motor function score on the CHOP INTEND that is >45 or that neuromuscular development milestones have been met; (3) determining that the patient exhibits a maximal inspiratory pressure that is >−50 cmH₂O on the ventilator; (4) determining that the patient exhibits a maximal expiratory pressure that is >40 cmH₂O on the ventilator; (5) determining that the patient exhibits a positive end-expiratory pressure that is cmH₂O on the ventilator; (6) determining that the patient exhibits a SpO₂ that is >94%; (7) determining that the patient exhibits a TcCO₂ that is within 35-45 mmHg; (8) determining that the patient exhibits an ETCO₂ that is within 35-45 mmHg; or (9) determining that the patient exhibits serum bicarbonate levels that are within 22-27 mEq/L. Readiness for daytime weaning off of mechanical ventilation may be assessed, for example, by determining: that the patient exhibits vital signs and a weight that are within the age-adjusted norms, that the patient exhibits a motor function score on the CHOP INTEND that is >45 or that neuromuscular development milestones have been met, and that the patient exhibits a maximal inspiratory pressure that is >−50 cmH₂O on the ventilator.

If, upon assessment of the aforementioned parameters, a physician of skill in the art determines that a patient is ready for daytime weaning off of mechanical ventilation, following daytime weaning off of mechanical ventilation to assess the patient's readiness for continuation of daytime weaning off of mechanical ventilation, a physician of skill in the art may analyze one or more of the following parameters: (1) determining that the patient exhibits a RR that is within the age-adjusted norms when respiration is monitored nocturnally; (2) determining that the patient exhibits no distress in the video recording of a respiratory sprinting trial; (3) determining that the patient exhibits a RR that is within the age-adjusted norm when a polysomnogram is performed with the tracheostomy open; (4) determining that the patient exhibits a TcCO₂ that is within 35-45 mmHg when respiration is monitored nocturnally; (5) an ETCO₂ that is within 35-45 mmHg when respiration is monitored nocturnally; (6) a SpO₂ that is >94 when respiration is monitored nocturnally; (7) determining that the patient exhibits an AHI that is <5 events/hour when a polysomnogram is performed with the tracheostomy open; (8) determining that the patient exhibits a TcCO₂ that is within 35-45 mmHg or that did not increases by 10 mmHg or greater above the awake baseline when a polysomnogram is performed with the tracheostomy open; (9) determining that the patient exhibits a petCO₂ or a ptcCO₂ that is <50 mmHg or that did not increase by greater than 10 mmHg from the awake baseline during sleep when a polysomnogram is performed with the tracheostomy open; (10) determining that the patient exhibits no intercostal retraction in the video recording of a respiratory sprinting trial; (11) determining that the patient exhibits no tachypnea in the video recording of a respiratory sprinting trial; (12) determining that the patient exhibits no respiratory paradox in the video recording of a respiratory sprinting trial; (13) determining that the patient exhibits no phase delay in the video recording of a respiratory sprinting trial; (14) determining that the patient exhibits an SpO₂ that is <94% or that does not differ by greater than 3% from baseline in the video recording of a respiratory sprinting trial; or (15) determining that the patient exhibits a TcCO₂ that is >45 mmHg or that did not increase by 10 mmHg or greater above the awake baseline in the video recording of a respiratory sprinting trial. Readiness for the continuation of daytime weaning off of mechanical ventilation may be assessed, for example, by determining: that the patient exhibits a RR that is within the age-adjusted norms when respiration is monitored nocturnally, that the patient exhibits no distress in the video recording of a respiratory sprinting trial, and that the patient exhibits a TcCO₂ that is within 35-45 mmHg when respiration is monitored nocturnally.

Alternatively, to assess the patient's readiness for daytime weaning off of mechanical ventilation following administration of a therapeutic agent described above, a physician of skill in the art may analyze all of the following parameters: (1) determining that the patient exhibits vital signs and a weight that are within the age-adjusted norms; (2) determining that the patient exhibits a motor function score on the Children's Hospital of Philadelphia Infant Test of Neuromuscular Disorders (CHOP INTEND) that is >45 or that neuromuscular development milestones have been met; (3) determining that the patient exhibits a maximal inspiratory pressure that is >−50 cmH₂O on the ventilator; (4) determining that the patient exhibits a maximal expiratory pressure that is >40 cmH₂O on the ventilator; (5) determining that the patient exhibits a positive end-expiratory pressure that is cmH₂O on the ventilator; (6) determining that the patient exhibits a SpO₂ that is >94%; (7) determining that the patient exhibits a TcCO₂ that is within 35-45 mmHg; (8) determining that the patient exhibits an ETCO₂ that is within 35-45 mmHg; and (9) determining that the patient exhibits serum bicarbonate levels that are within 22-27 mEq/L (FIG. 3; box outlined in solid bold). Readiness for daytime weaning off of mechanical ventilation may be assessed, for example, by determining: that the patient exhibits vital signs and a weight that are within the age-adjusted norms, that the patient exhibits a motor function score on the CHOP INTEND that is >45 or that neuromuscular development milestones have been met, that the patient exhibits a maximal inspiratory pressure that is >−50 cmH₂O on the ventilator, determining that the patient exhibits a maximal expiratory pressure that is >40 cmH₂O on the ventilator, determining that the patient exhibits a positive end-expiratory pressure that is cmH₂O on the ventilator, determining that the patient exhibits a SpO₂ that is >94%, determining that the patient exhibits a TcCO₂ that is within 35-45 mmHg, determining that the patient exhibits an ETCO₂ that is within 35-45 mmHg, and determining that the patient exhibits serum bicarbonate levels that are within 22-27 mEq/L.

If, upon assessment of the aforementioned parameters, a physician of skill in the art determines that a patient is ready for daytime weaning off of mechanical ventilation, following daytime weaning off of mechanical ventilation to assess the patient's readiness for continuation of daytime weaning off of mechanical ventilation, a physician of skill in the art may analyze all of the following parameters: (1) determining that the patient exhibits a RR that is within the age-adjusted norms when respiration is monitored nocturnally; (2) determining that the patient exhibits no distress in the video recording of a respiratory sprinting trial; (3) determining that the patient exhibits a RR that is within the age-adjusted norm when a polysomnogram is performed with the tracheostomy open; (4) determining that the patient exhibits a TcCO₂ that is within 35-45 mmHg when respiration is monitored nocturnally; (5) an ETCO₂ that is within 35-45 mmHg when respiration is monitored nocturnally; (6) a SpO₂ that is >94 when respiration is monitored nocturnally; (7) determining that the patient exhibits an AHI that is <5 events/hour when a polysomnogram is performed with the tracheostomy open; (8) determining that the patient exhibits a TcCO₂ that is within 35-45 mmHg or that did not increases by 10 mmHg or greater above the awake baseline when a polysomnogram is performed with the tracheostomy open; (9) determining that the patient exhibits a petCO₂ or a ptcCO₂ that is <50 mmHg or that did not increase by greater than 10 mmHg from the awake baseline during sleep when a polysomnogram is performed with the tracheostomy open; (10) determining that the patient exhibits no intercostal retraction in the video recording of a respiratory sprinting trial; (11) determining that the patient exhibits no tachypnea in the video recording of a respiratory sprinting trial; (12) determining that the patient exhibits no respiratory paradox in the video recording of a respiratory sprinting trial; (13) determining that the patient exhibits no phase delay in the video recording of a respiratory sprinting trial; (14) determining that the patient exhibits an SpO₂ that is <94% or that does not differ by greater than 3% from awake baseline in the video recording of a respiratory sprinting trial; and (15) determining that the patient exhibits a TcCO₂ that is >45 mmHg or that did not increase by 10 mmHg or greater above the awake baseline in the video recording of a respiratory sprinting trial (FIG. 3; boxes outlined in dashed bold). Readiness for the continuation of daytime weaning off of mechanical ventilation may be assessed, for example, by determining: that the patient exhibits a RR that is within the age-adjusted norms when respiration is monitored nocturnally, that the patient exhibits no distress in the video recording of a respiratory sprinting trial, that the patient exhibits a TcCO₂ that is within 35-45 mmHg when respiration is monitored nocturnally, that the patient exhibits an ETCO₂ that is within 35-45 mmHg when respiration is monitored nocturnally, determining that the patient exhibits a SpO₂ that is >94 when respiration is monitored nocturnally, that the patient exhibits no intercostal retraction in the video recording of a respiratory sprinting trial, that the patient exhibits no tachypnea in the video recording of a respiratory sprinting trial, that the patient exhibits no respiratory paradox in the video recording of a respiratory sprinting trial, that the patient exhibits no phase delay in the video recording of a respiratory sprinting trial, that the patient exhibits an SpO₂ that is <94% or that does not differ by greater than 3% from awake baseline in the video recording of a respiratory sprinting trial, and that the patient exhibits a TcCO₂ that is >45 mmHg or that did not increase by 10 mmHg or greater above the awake baseline in the video recording of a respiratory sprinting trial. If a polysomnogram were performed with the tracheostomy open, the readiness for the continuation of daytime weaning off of mechanical ventilation may be assessed, for example, by additionally determining: that the patient exhibits a RR that is within the age-adjusted norm when a polysomnogram is performed with the tracheostomy open, that the patient exhibits an AHI that is <5 events/hour when a polysomnogram is performed with the tracheostomy open, that the patient exhibits a TcCO₂ that is within 35-45 mmHg or that did not increases by 10 mmHg or greater above the awake baseline when a polysomnogram is performed with the tracheostomy open, and that the patient exhibits a petCO₂ or a ptcCO₂ that is <50 mmHg or that did not increase by greater than 10 mmHg from the awake baseline during sleep when a polysomnogram is performed with the tracheostomy open.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of weaning a human patient that is on mechanical ventilation and that has X-linked myotubular myopathy (XLMTM) off of mechanical ventilation, wherein the patient has previously been administered a therapeutically effective amount of a viral vector comprising a transgene encoding Myotubularin 1 (MTM1), the method comprising:
a. determining that the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a saturation of room air oxygen (SpO₂) of about 94% or more, (v) a transcutaneous CO₂ (TcCO₂) of from about 35 mmHg to about 45 mmHg, (vi) an End Tidal CO₂ (petCO₂) of from about 35 mmHg to about 45 mmHg, and (vii) a serum bicarbonate level of from about 22 mEq/L to about 27 mEq/L; and
b. weaning the patient off of mechanical ventilation during daytime hours.

2. The method of claim 1, wherein the method comprises determining that the patient exhibits a maximal inspiratory pressure of about 50 cmH₂O or more on a ventilator.

3. The method of claim 1 or 2, wherein the method comprises determining that the patient exhibits a maximal expiratory pressure of about 40 cmH₂O or more on a ventilator.

4. The method of any one of claims 1-3, wherein the method comprises determining that the patient exhibits a positive end-expiratory pressure of about 5 cmH₂O or less on a ventilator.

5. The method of any one of claims 1-4, wherein the method comprises determining that the patient exhibits a SpO₂ of about 94% or more.

6. The method of any one of claims 1-5, wherein the method comprises determining that the patient exhibits a TcCO₂ of from about 35 mmHg to about 45 mmHg.

7. The method of any one of claims 1-6, wherein the method comprises determining that the patient exhibits a petCO₂ of from about 35 mmHg to about 45 mmHg.

8. The method of any one of claims 1-7, wherein the method comprises determining that the patient exhibits a serum bicarbonate level of from about 22 mEq/L to about 27 mEq/L.

9. The method of any one of claims 1-8, wherein the method further comprises determining that the patient exhibits vital signs and a weight that are within age-adjusted norms.

10. The method of any one of claims 1-9, wherein the method further comprises determining that the patient exhibits a motor function score on the Children's Hospital of Philadelphia Infant Test of Neuromuscular Disorders (CHOP INTEND) of greater than 45 or that neuromuscular development milestones have been met.

11. The method of any one of claims 1-10, wherein the method further comprises:
c. determining that the patient exhibits one or more of (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an apnea-hypopnea index (AHI) of less than 5 events/hour as assessed by polysomnogram (PSG) performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a partial pressure of CO₂ (ptcCO₂) that does not increase by 10 mmHg or more during sleep relative to the patient's awake baseline as assessed by PSG performed with a tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TcCO₂ of greater than 45 mmHg as assessed by a video recording of a respiratory sprinting trial, and (xvi) a TCO₂ that does not increase by 10 mmHg or more relative to the patient's awake baseline as assessed by a video recording of a respiratory sprinting trial; and
d. continuing to wean the patient off of mechanical ventilation during daytime hours.

12. The method of claim 11, wherein the method comprises determining that the patient exhibits a TcCO₂ of from about 35 mmHg to about 45 mmHg as assessed by nocturnal respiration monitoring.

13. The method of claim 11 or 12, wherein the method comprises determining that the patient exhibits a petCO₂ of from about 35 mmHg to about 45 mmHg as assessed by nocturnal respiration monitoring.

14. The method of any one of claims 11-13, wherein the method comprises determining that the patient exhibits an SpO₂ of about 94% or more as assessed by nocturnal respiration monitoring.

15. The method of any one of claims 11-14, wherein the method comprises determining that the patient exhibits an AHI of less than 5 events/hour as assessed by PSG performed with an open tracheostomy.

16. The method of any one of claims 11-15, wherein the method comprises determining that the patient exhibits a TcCO₂ of from about 35 mmHg to about 45 mmHg as assessed by PSG performed with an open tracheostomy.

17. The method of any one of claims 11-16, wherein the method comprises determining that the patient exhibits a TcCO₂ that does not increase by 10 mmHg or more relative to the patient's awake baseline as assessed by PSG performed with an open tracheostomy.

18. The method of any one of claims 11-17, wherein the method comprises determining that the patient exhibits a petCO₂ or a ptcCO₂ of less than 50 mmHg as assessed by PSG performed with an open tracheostomy.

19. The method of any one of claims 11-18, wherein the method comprises determining that the patient exhibits a petCO₂ or ptcCO₂ that does not increase by 10 mmHg or more during sleep relative to the patient's awake baseline as assessed by PSG performed with a tracheostomy.

20. The method of any one of claims 11-19, wherein the method comprises determining that the patient exhibits no intercostal retraction in a video recording of a respiratory sprinting trial.

21. The method of any one of claims 11-20, wherein the method comprises determining that the patient exhibits no tachypnea in a video recording of a respiratory sprinting trial.

22. The method of any one of claims 11-21, wherein the method comprises determining that the patient exhibits no respiratory paradox in a video recording of a respiratory sprinting trial.

23. The method of any one of claims 11-22, wherein the method comprises determining that the patient exhibits no phase delay in a video recording of a respiratory sprinting trial.

24. The method of any one of claims 11-23, wherein the method comprises determining that the patient exhibits an SpO₂ of less than 94% as assessed by a video recording of a respiratory sprinting trial.

25. The method of any one of claims 11-24, wherein the method comprises determining that the patient exhibits an SpO₂ that does not differ by greater than 3% relative to the patient's awake baseline as assessed by a video recording of a respiratory sprinting trial.

26. The method of any one of claims 11-25, wherein the method comprises determining that the patient exhibits a TCO₂ of greater than 45 mmHg as assessed by a video recording of a respiratory sprinting trial.

27. The method of any one of claims 11-26, wherein the method comprises determining that the patient exhibits a TCO₂ that does not increase by 10 mmHg or more relative to the patient's awake baseline as assessed by a video recording of a respiratory sprinting trial.

28. The method of any one of claims 11-27, wherein the method further comprises determining that the patient exhibits a respiration rate that is within age-adjusted norms as assessed by nocturnal respiration monitoring.

29. The method of any one of claims 11-28, wherein the method further comprises determining that the patient exhibits no distress in a video recording of a respiratory sprinting trial.

30. The method of any one of claims 11-29, wherein the method further comprises determining that the patient exhibits a respiration rate that is within age-adjusted norm as assessed by PSG performed with an open tracheostomy.

31. A method of weaning a human patient that is on mechanical ventilation and that has XLMTM off of mechanical ventilation, wherein the patient has previously been administered a therapeutically effective amount of a viral vector comprising a transgene encoding MTM1, the method comprising:
a. measuring in the patient one or more of (i) maximal inspiratory pressure on a ventilator, (ii) maximal expiratory pressure on a ventilator, (iii) positive end-expiratory pressure on a ventilator, (iv) SpO₂ level, (v) TcCO₂ level, (vi) petCO₂ level, and (vii) serum bicarbonate level; and
b. weaning the patient off of mechanical ventilation during daytime hours if the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, and (vii) a serum bicarbonate level of from about 22 mEq/L to about 27 mEq/L.

32. The method of claim 31, wherein the method comprises determining that the patient exhibits a maximal inspiratory pressure of about 50 cmH₂O or more on a ventilator.

33. The method of claim 31 or 32, wherein the method comprises determining that the patient exhibits a maximal expiratory pressure of about 40 cmH₂O or more on a ventilator.

34. The method of any one of claims 31-33, wherein the method comprises determining that the patient exhibits a positive end-expiratory pressure of about 5 cmH₂O or less on a ventilator.

35. The method of any one of claims 31-34, wherein the method comprises determining that the patient exhibits a SpO₂ of about 94% or more.

36. The method of any one of claims 31-35, wherein the method comprises determining that the patient exhibits a TcCO₂ of from about 35 mmHg to about 45 mmHg.

37. The method of any one of claims 31-36, wherein the method comprises determining that the patient exhibits a petCO₂ of from about 35 mmHg to about 45 mmHg.

38. The method of any one of claims 31-37, wherein the method comprises determining that the patient exhibits a serum bicarbonate level of from about 22 mEq/L to about 27 mEq/L.

39. The method of any one of claims 31-38, wherein the method further comprises determining that the patient exhibits vital signs and a weight that are within age-adjusted norms.

40. The method of any one of claims 31-39, wherein the method further comprises determining that the patient exhibits a motor function score on the CHOP INTEND of greater than 45 or that neuromuscular development milestones have been met.

41. A method of treating a human patient that has X-linked myotubular myopathy (XLMTM) and that is on mechanical ventilation, the method comprising:
a. administering to the patient a therapeutically effective amount of a viral vector comprising a transgene encoding myotubularin 1 (MTM1);
b. determining that the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a saturation of room air oxygen (SpO₂) of about 94% or more, (v) a transcutaneous CO₂ (TcCO₂) of from about 35 mmHg to about 45 mmHg, (vi) an End Tidal CO₂ (petCO₂) of from about 35 mmHg to about 45 mmHg, and (vii) a serum bicarbonate level of from about 22 mEq/L to about 27 mEq/L; and
c. weaning the patient off of mechanical ventilation during daytime hours.

42. The method of claim 41, wherein the method comprises determining that the patient exhibits a maximal inspiratory pressure of about 50 cmH₂O or more on a ventilator.

43. The method of claim 41 or 42, wherein the method comprises determining that the patient exhibits a maximal expiratory pressure of about 40 cmH₂O or more on a ventilator.

44. The method of any one of claims 41-43, wherein the method comprises determining that the patient exhibits a positive end-expiratory pressure of about 5 cmH₂O or less on a ventilator.

45. The method of any one of claims 41-44, wherein the method comprises determining that the patient exhibits a SpO₂ of about 94% or more.

46. The method of any one of claims 41-45, wherein the method comprises determining that the patient exhibits a TcCO₂ of from about 35 mmHg to about 45 mmHg.

47. The method of any one of claims 41-46, wherein the method comprises determining that the patient exhibits a petCO₂ of from about 35 mmHg to about 45 mmHg.

48. The method of any one of claims 41-47, wherein the method comprises determining that the patient exhibits a serum bicarbonate level of from about 22 mEq/L to about 27 mEq/L.

49. The method of any one of claims 41-48, wherein the method further comprises determining that the patient exhibits vital signs and a weight that are within age-adjusted norms.

50. The method of any one of claims 41-49, wherein the method further comprises determining that the patient exhibits a motor function score on the Children's Hospital of Philadelphia Infant Test of Neuromuscular Disorders (CHOP INTEND) of greater than 45 or that neuromuscular development milestones have been met.

51. The method of any one of claims 41-50, wherein the method further comprises:
d. determining that the patient exhibits one or more of (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an apnea-hypopnea index (AHI) of less than 5 events/hour as assessed by polysomnogram (PSG) performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a partial pressure of CO₂ (ptcCO₂) of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with a tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TcCO₂ of greater than 45 mmHg as assessed by a video recording of a respiratory sprinting trial, and (xvi) a TcCO₂ that does not increase by 10 mmHg or more relative to the patient's awake baseline as assessed by a video recording of a respiratory sprinting trial; and
e. continuing to wean the patient off of mechanical ventilation during daytime hours.

52. The method of claim 51, wherein the method comprises determining that the patient exhibits a TcCO₂ of from about 35 mmHg to about 45 mmHg as assessed by nocturnal respiration monitoring.

53. The method of claim 51 or 52, wherein the method comprises determining that the patient exhibits a petCO₂ of from about 35 mmHg to about 45 mmHg as assessed by nocturnal respiration monitoring.

54. The method of any one of claims 51-53, wherein the method comprises determining that the patient exhibits an SpO₂ of about 94% or more as assessed by nocturnal respiration monitoring.

55. The method of any one of claims 51-54, wherein the method comprises determining that the patient exhibits an AHI of less than 5 events/hour as assessed by PSG performed with an open tracheostomy.

56. The method of any one of claims 51-55, wherein the method comprises determining that the patient exhibits a TcCO₂ of from about 35 mmHg to about 45 mmHg as assessed by PSG performed with an open tracheostomy.

57. The method of any one of claims 51-56, wherein the method comprises determining that the patient exhibits a TcCO₂ that does not increase by 10 mmHg or more relative to the patient's awake baseline as assessed by PSG performed with an open tracheostomy.

58. The method of any one of claims 51-57, wherein the method comprises determining that the patient exhibits a petCO₂ or a ptcCO₂ of less than 50 mmHg as assessed by PSG performed with an open tracheostomy.

59. The method of any one of claims 51-58, wherein the method comprises determining that the patient exhibits a petCO₂ or ptcCO₂ that does not increase by 10 mmHg or more during sleep relative to the patient's awake baseline as assessed by PSG performed with a tracheostomy.

60. The method of any one of claims 51-59, wherein the method comprises determining that the patient exhibits no intercostal retraction in a video recording of a respiratory sprinting trial.

61. The method of any one of claims 51-60, wherein the method comprises determining that the patient exhibits no tachypnea in a video recording of a respiratory sprinting trial.

62. The method of any one of claims 51-61, wherein the method comprises determining that the patient exhibits no respiratory paradox in a video recording of a respiratory sprinting trial.

63. The method of any one of claims 51-62, wherein the method comprises determining that the patient exhibits no phase delay in a video recording of a respiratory sprinting trial.

64. The method of any one of claims 51-63, wherein the method comprises determining that the patient exhibits an SpO₂ of less than 94% as assessed by a video recording of a respiratory sprinting trial.

65. The method of any one of claims 51-64, wherein the method comprises determining that the patient exhibits an SpO₂ that does not differ by greater than 3% relative to the patient's awake baseline as assessed by a video recording of a respiratory sprinting trial.

66. The method of any one of claims 51-65, wherein the method comprises determining that the patient exhibits a TcCO₂ of greater than 45 mmHg as assessed by a video recording of a respiratory sprinting trial.

67. The method of any one of claims 51-66, wherein the method comprises determining that the patient exhibits a TcCO₂ that does not increase by 10 mmHg or more relative to the patient's awake baseline as assessed by a video recording of a respiratory sprinting trial.

68. The method of any one of claims 51-67, wherein the method further comprises determining that the patient exhibits a respiration rate that is within age-adjusted norms as assessed by nocturnal respiration monitoring.

69. The method of any one of claims 51-68, wherein the method further comprises determining that the patient exhibits no distress in a video recording of a respiratory sprinting trial.

70. The method of any one of claims 51-69, wherein the method further comprises determining that the patient exhibits a respiration rate that is within age-adjusted norm as assessed by PSG performed with an open tracheostomy.

71. A method of treating a human patient that has XLMTM and that is on mechanical ventilation, the method comprising:
a. administering to the patient a therapeutically effective amount of a viral vector comprising a transgene encoding MTM1;
b. measuring in the patient one or more of (i) maximal inspiratory pressure on a ventilator, (ii) maximal expiratory pressure on a ventilator, (iii) positive end-expiratory pressure on a ventilator, (iv) SpO₂ level, (v) TcCO₂ level, (vi) petCO₂ level, and (vii) serum bicarbonate level; and
c. weaning the patient off of mechanical ventilation during daytime hours if the patient exhibits one or more of (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, and (vii) a serum bicarbonate level of from about 22 mEq/L to about 27 mEq/L.

72. The method of claim 71, wherein the method comprises determining that the patient exhibits a maximal inspiratory pressure of about 50 cmH₂O or more on a ventilator.

73. The method of claim 71 or 72, wherein the method comprises determining that the patient exhibits a maximal expiratory pressure of about 40 cmH₂O or more on a ventilator.

74. The method of any one of claims 71-73, wherein the method comprises determining that the patient exhibits a positive end-expiratory pressure of about 5 cmH₂O or less on a ventilator.

75. The method of any one of claims 71-74, wherein the method comprises determining that the patient exhibits a SpO₂ of about 94% or more.

76. The method of any one of claims 71-75, wherein the method comprises determining that the patient exhibits a TcCO₂ of from about 35 mmHg to about 45 mmHg.

77. The method of any one of claims 71-76, wherein the method comprises determining that the patient exhibits a petCO₂ of from about 35 mmHg to about 45 mmHg.

78. The method of any one of claims 71-77, wherein the method comprises determining that the patient exhibits a serum bicarbonate level of from about 22 mEq/L to about 27 mEq/L.

79. The method of any one of claims 71-78, wherein the method further comprises determining that the patient exhibits vital signs and a weight that are within age-adjusted norms.

80. The method of any one of claims 71-79, wherein the method further comprises determining that the patient exhibits a motor function score on the CHOP INTEND of greater than 45 or that neuromuscular development milestones have been met.

81. The method of any one of claims 71-80, wherein the weaning off of mechanical ventilation comprises a gradual reduction in ventilator support parameters including one or more of pressure, volume, and rate, followed by a progressive sprinting of off the ventilator, optionally wherein no more than one parameter of ventilator support is changed at a time.

82. The method of any one of claims 71-81, wherein upon administering the viral vector to the patient, the patient exhibits a change from baseline in hours of ventilation support over time, optionally wherein the patient exhibits the change from baseline in hours of ventilation support over time by about 24 weeks after administration of the viral vector to the patient.

83. The method of any one of claims 71-82, wherein upon administering the viral vector to the patient, the patient achieves functionally independent sitting for at least 30 seconds, optionally wherein the patient achieves the functionally independent slitting by about 24 weeks after administration of the viral vector to the patient.

84. The method of any one of claims 71-83, wherein upon administering the viral vector to the patient, the patient displays a reduction in required ventilator support to about 16 hours or less per day, optionally wherein the patient displays the reduction in required ventilator support by about 24 weeks after administration of the viral vector to the patient.

85. The method of any one of claims 71-84, wherein upon administering the viral vector to the patient, the patient displays a change from baseline on the CHOP INTEND, optionally wherein the patient displays the change from baseline on the CHOP INTEND by about 24 weeks after administration of the viral vector to the patient.

86. The method of any one of claims 71-85, wherein upon administering the viral vector to the patient, the patient displays a change from baseline in maximal inspiratory pressure, optionally wherein the patient displays the change from baseline in maximal inspiratory pressure by about 24 weeks after administration of the viral vector to the patient.

87. The method of any one of claims 71-86, wherein upon administering the viral vector to the patient, the patient displays a change from baseline in quantitative analysis of myotubularin expression in a muscle biopsy, optionally wherein the patient displays the change from baseline in quantitative analysis of myotubularin expression in a muscle biopsy by about 24 weeks after administration of the viral vector to the patient.

88. The method of any one of claims 71-87, wherein the transgene encoding MTM1 is operably linked to a muscle specific promoter.

89. The method of claim 88, wherein the muscle specific promotor is a desmin promoter, a phosphoglycerate kinase (PGK) promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3 (PITX3).

90. The method of claim 89, wherein the muscle specific promoter is a desmin promoter.

91. The method of any one of claims 1-90, wherein the viral vector is selected from the group consisting of adeno-associated virus (AAV), adenovirus, lentivirus, retrovirus, poxvirus, baculovirus, herpes simplex virus, vaccinia virus, and a synthetic virus.

92. The method of claim 91, wherein the viral vector is an AAV.

93. The method of claim 92, wherein the AAV is an AAV1, AAV2, AAV3, AAV4, AAV5, AAV6, AAV7, AAV8, AAV9, AAVrh10, or AAVrh74 serotype.

94. The method of claim 91, wherein the viral vector is a pseudotyped AAV.

95. The method of claim 94, wherein the pseudotyped AAV is AAV2/8 or AAV2/9, optionally wherein the pseudotyped AAV is AAV2/8.

96. The method of any one of claims 1-95, wherein the viral vector is resamirigene bilparvovec.

97. A method of treating a human patient that has XLMTM and that is on mechanical ventilation, the method comprising:
a. administering to the patient a therapeutically effective amount of an AAV2/8 viral vector comprising a transgene encoding MTM1 operably linked to a desmin promotor;
b. determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met;
c. weaning the patient off of mechanical ventilation during daytime hours;
d. determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) no intercostal retraction in a video recording of a respiratory sprinting trial, (v) no tachypnea in a video recording of a respiratory sprinting trial, (vi) no respiratory paradox in a video recording of a respiratory sprinting trial, (vii) no phase delay in a video recording of a respiratory sprinting trial, (viii) an SpO₂ of less than 94% as assessed by a video recording of a respiratory sprinting trial, (ix) an SpO₂ that does not differ by greater than 3% relative to the patient's awake baseline as assessed by a video recording of a respiratory sprinting trial, (x) a TCO₂ of greater than 45 mmHg as assessed by a video recording of a respiratory sprinting trial, (xi) a TCO₂ that does not increase by 10 mmHg or more relative to the patient's awake baseline as assessed by a video recording of a respiratory sprinting trial, (xii) a respiration rate that is within age-adjusted norms as assessed by nocturnal respiration monitoring, (xiii) no distress in a video recording of a respiratory sprinting trial, and (xiv) a respiration rate that is within age-adjusted norm as assessed by PSG performed with an open tracheostomy; and
e. continuing to wean the patient off of mechanical ventilation during daytime hours.

98. A method of treating a human patient that has XLMTM and that is on mechanical ventilation, the method comprising:
a. administering to the patient a therapeutically effective amount of an AAV2/8 viral vector comprising a transgene encoding MTM1 operably linked to a desmin promotor;
b. determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met;
c. weaning the patient off of mechanical ventilation during daytime hours;
d. determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an AHI of less than 5 events/hour as assessed by PSG performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with a tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a TCO₂ of greater than 45 mmHg as assessed by a video recording of a respiratory sprinting trial, (xvi) a TCO₂ that does not increase by 10 mmHg or more relative to the patient's awake baseline as assessed by a video recording of a respiratory sprinting trial; (xvii) a respiration rate that is within age-adjusted norms as assessed by nocturnal respiration monitoring, (xviii) no distress in a video recording of a respiratory sprinting trial, and (xix) a respiration rate that is within age-adjusted norm as assessed by PSG performed with an open tracheostomy; and
e. continuing to wean the patient off of mechanical ventilation during daytime hours.

99. A method of weaning a human patient that is on mechanical ventilation and that has XLMTM off of mechanical ventilation, wherein the patient has previously been administered a therapeutically effective amount of an AAV2/8 viral vector comprising a transgene encoding MTM1 operably linked to a desmin promotor, the method comprising:
a. determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met;
b. weaning the patient off of mechanical ventilation during daytime hours;
c. determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) no intercostal retraction in a video recording of a respiratory sprinting trial, (v) no tachypnea in a video recording of a respiratory sprinting trial, (vi) no respiratory paradox in a video recording of a respiratory sprinting trial, (vii) no phase delay in a video recording of a respiratory sprinting trial, (viii) an SpO₂ of less than 94% as assessed by a video recording of a respiratory sprinting trial, (ix) an SpO₂ that does not differ by greater than 3% relative to the patient's awake baseline as assessed by a video recording of a respiratory sprinting trial, (x) a TCO₂ of greater than 45 mmHg as assessed by a video recording of a respiratory sprinting trial, (xi) a TCO₂ that does not increase by 10 mmHg or more relative to the patient's awake baseline as assessed by a video recording of a respiratory sprinting trial, (xii) a respiration rate that is within age-adjusted norms as assessed by nocturnal respiration monitoring, (xiii) no distress in a video recording of a respiratory sprinting trial, and (xiv) a respiration rate that is within age-adjusted norm as assessed by PSG performed with an open tracheostomy; and
d. continuing to wean the patient off of mechanical ventilation during daytime hours.

100. A method of weaning a human patient that is on mechanical ventilation and that has XLMTM off of mechanical ventilation, wherein the patient has previously been administered a therapeutically effective amount of an AAV2/8 viral vector comprising a transgene encoding MTM1 operably linked to a desmin promotor, the method comprising:
a. determining that the patient exhibits (i) a maximal inspiratory pressure of about 50 cmH₂O or more on a ventilator, (ii) a maximal expiratory pressure of about 40 cmH₂O or more on a ventilator, (iii) a positive end-expiratory pressure of about 5 cmH₂O or less on a ventilator, (iv) a SpO₂ of about 94% or more, (v) a TcCO₂ of from about 35 mmHg to about 45 mmHg, (vi) a petCO₂ of from about 35 mmHg to about 45 mmHg, (vii) a serum bicarbonate level of from about 22 mEq/L to about 27 mEq/L, (viii) vital signs and a weight that are within age-adjusted norms, and (ix) a motor function score on the CHOP INTEND of greater than 45 or that neuromuscular development milestones have been met;
b. weaning the patient off of mechanical ventilation during daytime hours;
c. determining that the patient exhibits (i) a TcCO₂ of from about 35 mmHg to about 45 mmHg as assessed by nocturnal respiration monitoring, (ii) a petCO₂ of from about 35 mmHg to about 45 mmHg as assessed by nocturnal respiration monitoring, (iii) an SpO₂ of about 94% or more as assessed by nocturnal respiration monitoring, (iv) an AHI of less than 5 events/hour as assessed by PSG performed with an open tracheostomy, (v) a TcCO₂ of from about 35 mmHg to about 45 mmHg as assessed by PSG performed with an open tracheostomy, (vi) a TcCO₂ that does not increase by 10 mmHg or more relative to the patient's awake baseline as assessed by PSG performed with an open tracheostomy, (vii) a petCO₂ or a ptcCO₂ of less than 50 mmHg as assessed by PSG performed with an open tracheostomy, (viii) a petCO₂ or a ptcCO₂ that does not increase by 10 mmHg or more during sleep relative to the patient's awake baseline as assessed by PSG performed with a tracheostomy, (ix) no intercostal retraction in a video recording of a respiratory sprinting trial, (x) no tachypnea in a video recording of a respiratory sprinting trial, (xi) no respiratory paradox in a video recording of a respiratory sprinting trial, (xii) no phase delay in a video recording of a respiratory sprinting trial, (xiii) an SpO₂ of less than 94% as assessed by a video recording of a respiratory sprinting trial, (xiv) an SpO₂ that does not differ by greater than 3% relative to the patient's awake baseline as assessed by a video recording of a respiratory sprinting trial, (xv) a T0002 of greater than 45 mmHg as assessed by a video recording of a respiratory sprinting trial, (xvi) a TcCO₂ that does not increase by 10 mmHg or more relative to the patient's awake baseline as assessed by a video recording of a respiratory sprinting trial; (xvii) a respiration rate that is within age-adjusted norms as assessed by nocturnal respiration monitoring, (xviii) no distress in a video recording of a respiratory sprinting trial, and (xix) a respiration rate that is within age-adjusted norm as assessed by PSG performed with an open tracheostomy; and
d. continuing to wean the patient off of mechanical ventilation during daytime hours.
